(19) United States
(12) Patent Application Publication
(10) Pub. No.: US 2026/0159492 A1
(43) Pub. Date: Jun. 11, 2026

(54) ESTER COMPOUND, AND ESTER COMPOUND COMPOSITION FOR RESIN RAW MATERIAL

(71) Applicant: HONSHU CHEMICAL INDUSTRY CO., LTD., Chuo-ku, Tokyo (JP)

(72) Inventors: Daichi OKAMURA, Wakayama-shi (JP); Takahiro ASAEDA, Wakayama-shi (JP); Yuma SHIBASAKI, Wakayama-shi (JP)

(21) Appl. No.: 19/129,607

(22) PCT Filed: Dec. 6, 2023
(86) PCT No.: PCT/JP2023/043565

§ 371 Date: May 13, 2025

(30) Foreign Application Priority Data
Dec. 6, 2022 (JP) 2022-194763

(51) Int. Cl. C07D 307/68; C08G 59/24; C08G 59/32; C08G 59/42

(52) U.S. Cl. CPC C07D 307/68; C08G 59/24; C08G 59/3218; C08G 59/4238

(57) ABSTRACT

An object is to provide a curing agent that has good handleability and gives a cured product excellent in heat resistance and dielectric properties. As a solution, an ester compound represented by general formula (3) is provided.

### TECHNICAL FIELD

The present invention relates to an ester compound, and an ester compound composition for a resin raw material. The invention particularly relates to an ester compound having furan rings at both ends of a linking group, and an ester compound composition for a resin raw material, the ester compound composition containing the ester compound.

### BACKGROUND ART

Epoxy resins are excellent in heat resistance, adhesiveness, water resistance, mechanical strength, and electrical characteristics and thus are used in various fields, including adhesives, coatings, composite materials, materials for civil engineering and construction, and insulating materials for electric and electronic components. Particularly in the electric and electronic fields, epoxy resins are widely used for insulating castings, laminating materials, sealing materials, etc.

Important properties required of epoxy resins used as materials for electric and electronic components include high heat resistance, low CTE, low dielectric constant, low dielectric loss tangent, low hygroscopicity, etc. Highly integrated semiconductor materials have recently been showing a trend toward higher multilayers, thinner insulating layers, and more complex structures and are required to have not only the foregoing properties but also a balance with various properties such as solvent solubility. There have recently been reports of techniques that seek to reduce polarization by esterifying a secondary hydroxy group formed as a result of the reaction between an epoxy group and a curing agent and improve low hygroscopicity and dielectric properties.

PTL 1 discloses a thermosetting composition that can achieve improved adhesion to a conductor layer while maintaining low hygroscopicity and dielectric properties by combining a thermosetting resin with a thermoplastic resin obtained by esterifying a secondary hydroxy group of an epoxy resin in a post-process, but the thermosetting composition has insufficient heat resistance.

### CITATION LIST

### Patent Literature

PTL 1: International Publication No. 2005/095517

### SUMMARY OF INVENTION

### Technical Problem

In view of the prior art, an object of the present invention is to provide a curing agent that has good handleability such as solvent solubility and that gives a cured product excellent in heat resistance and dielectric properties.

### Solution to Problem

To achieve the above object, the present inventors have conducted intensive studies and found that an ester compound of a bisphenol compound having a specific structure and a carboxylic acid having a furan ring dissolves in a solvent such as methyl ethyl ketone to offer good handleability, that the ester compound, when used as a curing agent, reacts with an epoxy resin to undergo chain-linking and then cures upon the chain-linking, and furthermore that a cured product obtained by esterifying a secondary hydroxy group is less polarized and has excellent dielectric properties and heat resistance, thereby completing the present invention.

The present invention is as follows.
1. An ester compound represented by general formula (3).

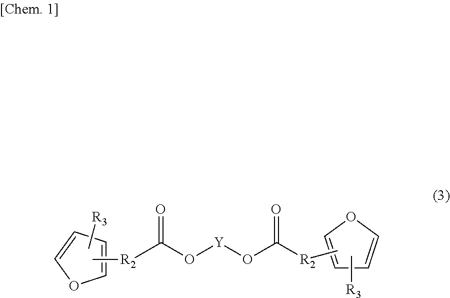

(In the formula, each R₂ independently represents a single bond or a divalent hydrocarbon group having 1 to 10 carbon atoms, each R₃ independently represents a hydrogen atom or an alkyl group having 1 to 6 carbon atoms, and Y represents a divalent group represented by general formula (3a) or general formula (3b).)

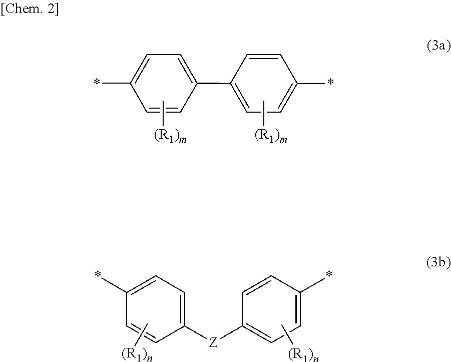

(In general formulas (3a) and (3b), each R₁ independently represents an alkyl group having 1 to 6 carbon atoms or an aryl group having 6 to 12 carbon atoms, and each * represents a bonding position. In general formula (3a), each m independently represents an integer of 1 to 4. In general formula (3b), each n independently represents 0 or an integer of 1 to 4, and Z represents a cycloalkylidene group having 7 to 20 carbon atoms.)
2. The ester compound according to 1., wherein each R₂ of the ester compound represented by general formula (3) is a single bond.
3. The ester compound according to 2., wherein furthermore, each R₃ of the ester compound represented by general formula (3) is a hydrogen atom.
4. The ester compound according to 3., wherein the ester compound represented by general formula (3) is compound (p-139), (p-142), (p-145), (p-148), (p-151), (p-154), or (p-172).

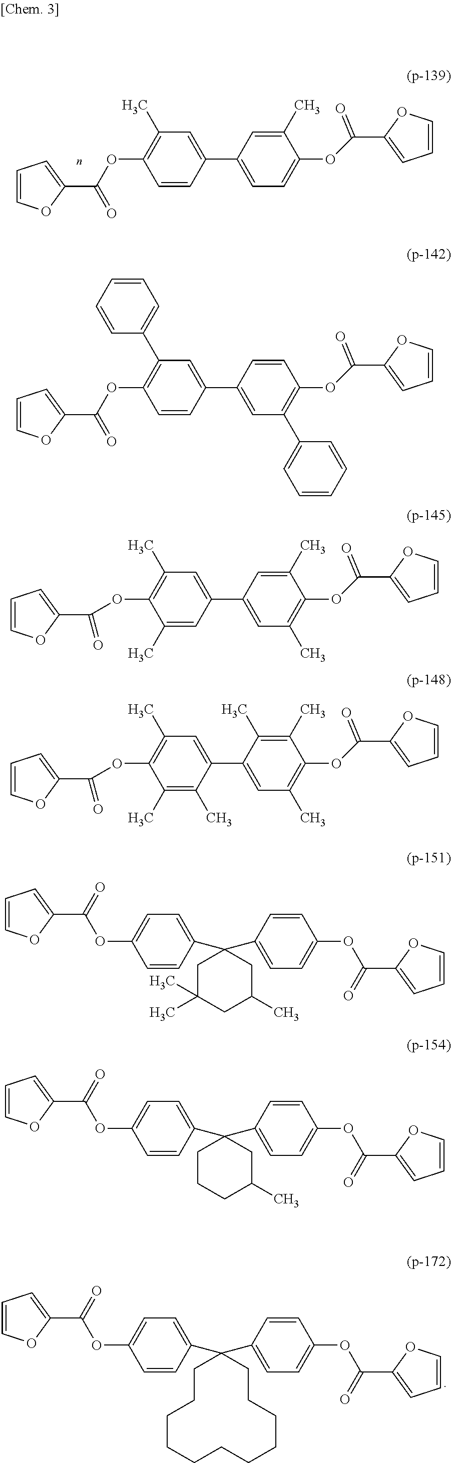

5. A crystal of the ester compound according to 4, the ester compound being compound (p-148).
6. The crystal of the ester compound according to 5., the ester compound being compound (p-148), wherein a maximum endothermic peak temperature determined by differential scanning calorimetry is in a range of 234° C. to 240° C.
7. A crystal of the ester compound according to 4, the ester compound being compound (p-151).
8. The crystal of the ester compound according to 7., the ester compound being compound (p-151), wherein a maximum endothermic peak temperature determined by differential scanning calorimetry is in a range of 180° C. to 188° C.
9. An ester compound composition for a resin raw material, containing the ester compound represented by general formula (3) according to 1, and an ester compound represented by general formula (2).

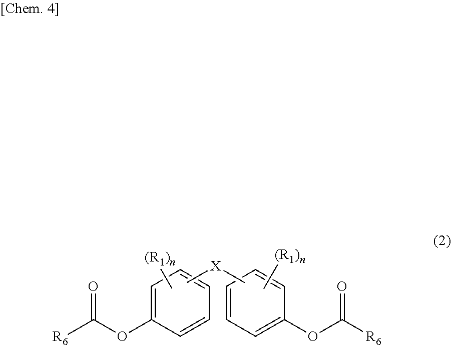

(In the formula, each R₁ independently represents an alkyl group having 1 to 6 carbon atoms or an aryl group having 6 to 12 carbon atoms, each Re independently represents a monovalent hydrocarbon group having 1 to 20 carbon atoms, X represents a single bond, an oxygen atom, a sulfur atom, a sulfonyl group, a carbonyl group, or a divalent group represented by general formula (1a), (1b), or (1c), and each n independently represents 0 or an integer of 1 to 4.)

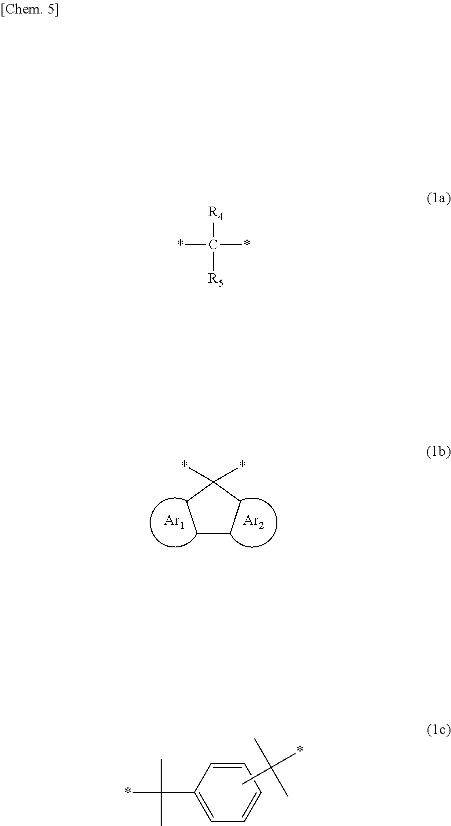

(In general formulas (1a), (1b), and (1c), R₄ and R₅ each independently represent a hydrogen atom, an alkyl group having 1 to 10 carbon atoms, an alkyl halide group having 1 to 10 carbon atoms, or an aryl group having 6 to 12 carbon atoms, R₄ and R₅ may be bonded to each other to together form a cycloalkylidene group having 5 to 20 carbon atoms, Ar₁ and Ar₂ represent an aryl group having 6 to 12 carbon atoms, and each * represents a bonding position.)
10. The ester compound composition for a resin raw material according to 9., wherein the ester compound represented by general formula (2) is contained in an amount of 0.1 to 400 parts by weight relative to 100 parts by weight of the ester compound represented by general formula (3).
11. An epoxy resin formed by reacting the ester compound represented by general formula (3) according to 1. with any one selected from an aromatic diglycidyl ether compound, a mixture containing an aromatic dihydroxy compound and epihalohydrin, and an epoxy group-containing phenoxy resin formed by polymerizing an aromatic dihydroxy compound and epihalohydrin.

### Advantageous Effects of Invention

The ester compound represented by general formula (3) according to the present invention in which a specific bisphenol is bonded to a carboxylic acid having a furan ring through an ester linkage dissolves in a solvent such as methyl ethyl ketone and thus has good handleability; the ester compound, when used as a curing agent, can react with an epoxy resin to undergo chain-linking and achieve curing; and furthermore, a cured product that is less polarized and has excellent dielectric properties and heat resistance can be provided by esterifying a secondary hydroxy group.

Thus, the ester compound represented by general formula (3) according to the present invention is applicable to various fields, including adhesives, composite materials, coatings, construction materials for civil engineering, and insulating materials for electric and electronic components, and can provide a cured product useful as an insulating casting, a laminating material, a sealing material, etc. particularly in the electric and electronic fields.

### BRIEF DESCRIPTION OF DRAWINGS

FIG. 1 shows a differential scanning calorimetry (DSC) curve of an ester compound (1-1) (compound (p-151)) obtained in Example 1.

FIG. 2 shows a differential scanning calorimetry (DSC) curve of an ester compound (1-2) (compound (p-28)) obtained in Example 2.

FIG. 3 shows a differential scanning calorimetry (DSC) curve of an ester compound (1-3) (compound (p-148)) obtained in Example 3.

### DESCRIPTION OF EMBODIMENTS

### <Curable Composition>

A curable composition according to the present invention contains an ester compound represented by general formula (1), and a thermosetting compound and/or a compound having a radical polymerizable substituent.

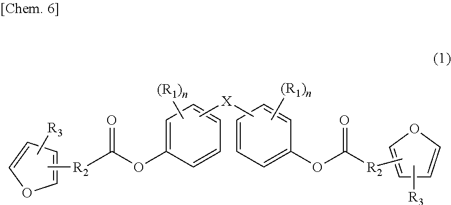

(In the formula, each R₁ independently represents an alkyl group having 1 to 6 carbon atoms or an aryl group having 6 to 12 carbon atoms, each R₂ independently represents a single bond or a divalent hydrocarbon group having 1 to 10 carbon atoms, each R₃ independently represents a hydrogen atom or an alkyl group having 1 to 6 carbon atoms, X represents a single bond, an oxygen atom, a sulfur atom, a sulfonyl group, a carbonyl group, or a divalent group represented by general formula (1a), (1b), or (1c), and each n independently represents 0 or an integer of 1 to 4.)

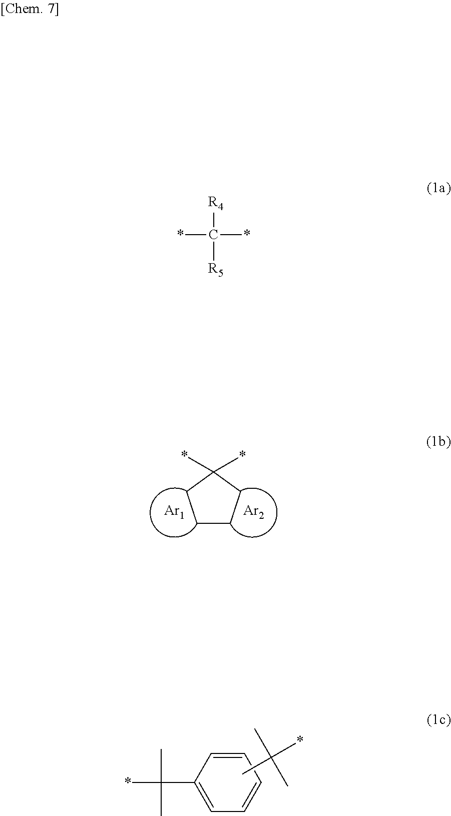

(In general formulas (1a), (1b), and (1c), R₄ and R₅ each independently represent a hydrogen atom, an alkyl group having 1 to 10 carbon atoms, an alkyl halide group having 1 to 10 carbon atoms, or an aryl group having 6 to 12 carbon atoms, R₄ and R₅ may be bonded to each other to together form a cycloalkylidene group having 5 to 20 carbon atoms, Ar₁ and Ar₂ represent an aryl group having 6 to 12 carbon atoms, and each * represents a bonding position.)

Due to the presence of the ester compound represented by general formula (1), cured products obtained by curing the curable composition have excellent heat resistance.

Among them, particularly for a secondary hydroxy group formed as a result of the reaction between an epoxy resin which is a thermosetting compound and a curing agent, the ester compound represented by general formula (1) reacts with the epoxy resin as a curing agent and esterifies the secondary hydroxy group, whereby a cured product that is less polarized and that has excellent dielectric properties is provided. Thus, the curable composition according to the present invention including an epoxy resin is useful and preferred because a cured product having these features is provided. Hence, the use of the ester compound represented by general formula (1) as a curing agent for an epoxy resin is preferred because a cured product having the above features is provided.

The ester compound represented by general formula (1) has a furan ring and thus can undergo a curing reaction also with the compound having a radical polymerizable substituent to provide a cured product.

The ester compound represented by general formula (1) has excellent solubility in solvents such as methyl ethyl ketone, cyclohexanone, N-methylpyrrolidone, toluene, propylene glycol monomethyl ether acetate, and ethyl acetate, particularly, methyl ethyl ketone, which is a solvent widely used in producing electronic components such as semiconductors, and thus is excellent in handleability.

### (Ester Compound Represented by General Formula (1))

Each R₁ in general formula (1) is preferably independently an alkyl group having 1 to 4 carbon atoms or a phenyl group, more preferably independently a methyl group or a phenyl group, particularly preferably a methyl group.

Each R₂ in general formula (1) is independently a single bond or a divalent hydrocarbon group having 1 to 10 carbon atoms. In the case of a divalent hydrocarbon group, specific examples include linear or branched alkylene groups having 1 to 10 carbon atoms or alkylene groups including a cyclic alkane, such as a methylene group, an ethylene group, a propane-1,2-diyl group, a propane-1,3-diyl group, a butane-1,4-diyl group, a pentane-1,5-diyl group, a hexane-1,6-diyl group, a cyclohexane-1,3-diyl group, and a cyclohexane-1,4-diyl group, alkylidene groups having 1 to 10 carbon atoms, such as an ethylidene group, a propylidene group, an isopropylidene group, a butylidene group, a cyclopentylidene group, and a cyclohexylidene group, and divalent groups including a benzene ring and having 1 to 10 carbon atoms, such as a phenylene group and groups represented by the following formulas.

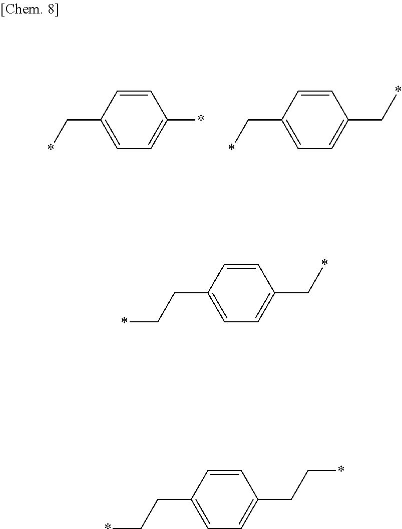

(In the formulas, * represents a bonding position.)

Of these, each R₂ is preferably independently a single bond, a linear or branched alkylene group having 1 to 10 carbon atoms, an alkylene group including a cyclic alkane, or an alkylidene group having 1 to 10 carbon atoms, more preferably independently a single bond, a linear or branched alkylene groups having 1 to 10 carbon atoms, or an alkylene group including a cyclic alkane, still more preferably independently a single bond, a linear or branched alkylene group having 1 to 6 carbon atoms, or an alkylene group including a cyclic alkane, particularly preferably a single bond. The ester compound represented by general formula (1) where each R₂ is a single bond is obtained from a biomass-derived material, and thus is suitable also because a curable composition with an improved biomass content is provided.

Each R₃ in general formula (1) is preferably independently a hydrogen atom or an alkyl group having 1 to 4 carbon atoms, more preferably independently a hydrogen atom or a methyl group, particularly preferably a hydrogen atom.

X in general formula (1) is preferably a single bond, a sulfonyl group, a carbonyl group, or a divalent group represented by general formula (1a), (1b), or (1c), more preferably a single bond or a divalent group represented by general formula (1a) or general formula (1b), still more preferably a single bond or a divalent group represented by general formula (1a), particularly preferably a single bond or a divalent group represented by general formula (1a) where R₄ and R₅ are bonded to each other to together form a cycloalkylidene group having 5 to 20 carbon atoms.

When X in general formula (1) is represented by general formula (1a), R₄ and R₅ are more preferably each independently a hydrogen atom, an alkyl group having 1 to 6 carbon atoms, an alkyl halide group having 1 to 6 carbon atoms, or an aryl group having 6 to 12 carbon atoms, still more preferably each independently a hydrogen atom, an alkyl group having 1 to 4 carbon atoms, a trifluoromethyl group, or an aryl group having 6 to 8 carbon atoms, particularly preferably each independently a hydrogen atom, an alkyl group having 1 to 4 carbon atoms, or a phenyl group.

R₄ and R₅ may be bonded to each other to together form a cycloalkylidene group having 5 to 20 carbon atoms, and R₄ and R₅ are more preferably in this form. The cycloalkylidene group having 5 to 20 carbon atoms may include a branched-chain alkyl group. The cycloalkylidene group preferably has 5 to 15 carbon atoms, more preferably has 6 to 12 carbon atoms, and particularly preferably has 6 to 9 carbon atoms. Specific examples of the cycloalkylidene group include a cyclopentylidene group (5 carbon atoms), a cyclohexylidene group (6 carbon atoms), a 3-methylcyclohexylidene group (7 carbon atoms), a 4-methylcyclohexylidene group (7 carbon atoms), a 3,3,5-trimethylcyclohexylidene group (9 carbon atoms), a cycloheptylidene group (7 carbon atoms), and a cyclododecanylidene group (12 carbon atoms). Preferred are a cyclohexylidene group (6 carbon atoms), a 3-methylcyclohexylidene group (7 carbon atoms), a 4-methylcyclohexylidene group (7 carbon atoms), a 3,3,5-trimethylcyclohexylidene group (9 carbon atoms), and a cyclododecanylidene group (12 carbon atoms), and more preferred are a cyclohexylidene group (6 carbon atoms), a 3,3,5-trimethylcyclohexylidene group (9 carbon atoms), and a cyclododecanylidene group (12 carbon atoms).

When X in general formula (1) is represented by general formula (1b), Ar₁ and Ar₂ are preferably each independently a benzene ring or a naphthalene ring, and Ar₁ and Ar₂ are more preferably both benzene rings. For example, when Ar₁ and Ar₂ are both benzene rings, the group represented by general formula (1b) is a fluorenylidene group.

When X in general formula (1) is represented by general formula (1c), it is preferably a divalent group that is 1,3-bis(isopropyl-2-yl)benzene or 1,4-bis(isopropyl-2-yl)benzene.

The positions of bonding of X in general formula (1) to two benzene rings are preferably each independently the ortho or para position, more preferably the para position, with respect to the oxygen atom bonded to each benzene ring.

Each n in general formula (1) is preferably independently 0, 2, or 3. When n is an integer of 1 to 4, R₁ is preferably bonded preferentially at the ortho position with respect to the oxygen atom bonded to each benzene ring.

As specific examples of the ester compound represented by general formula (1), compounds (p-1) to (p-171) having the following chemical structures are shown.

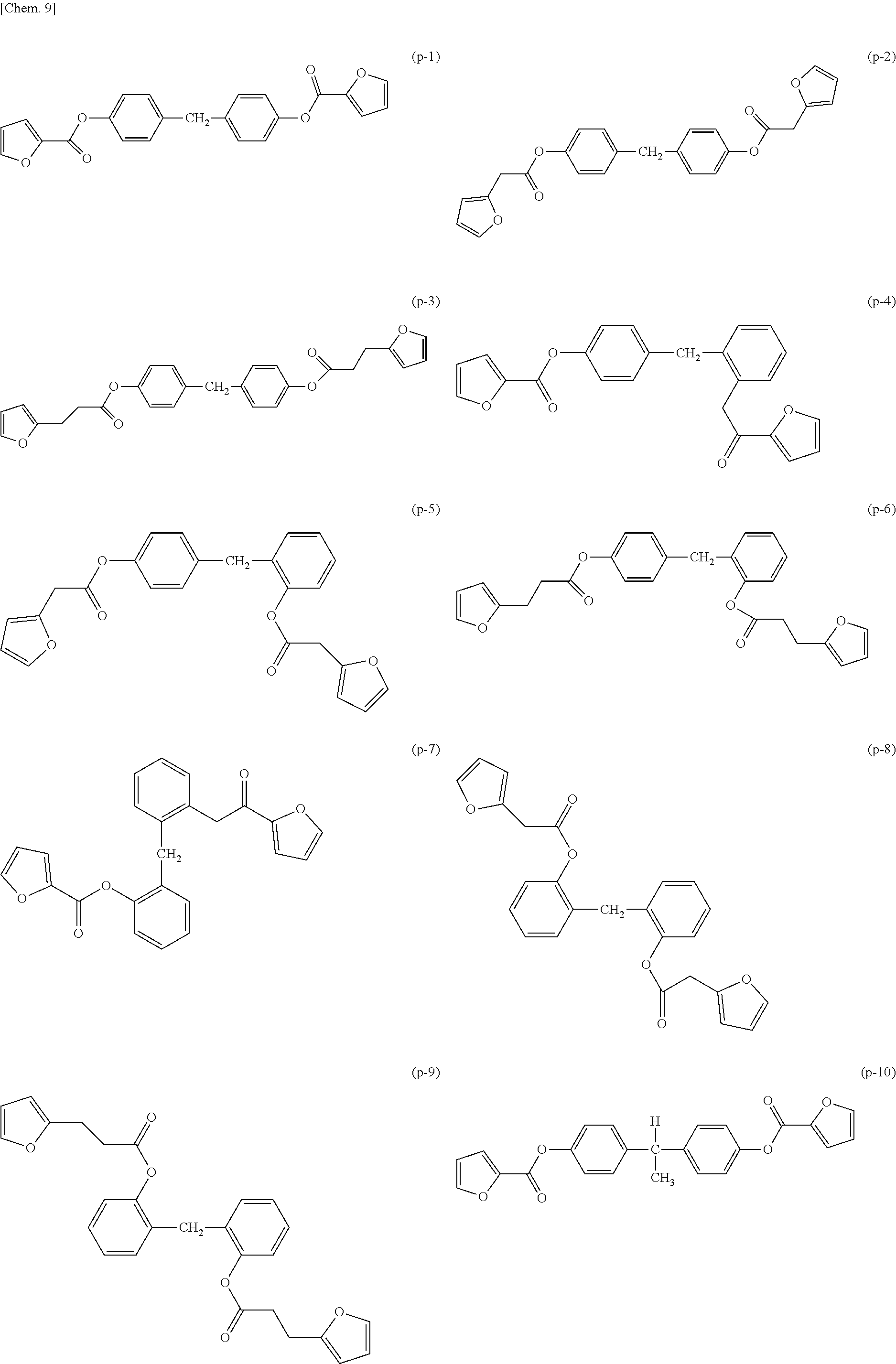

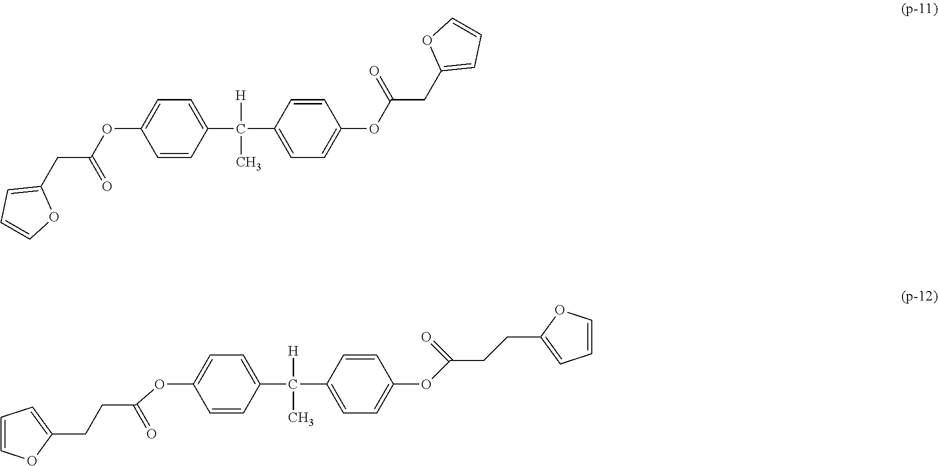

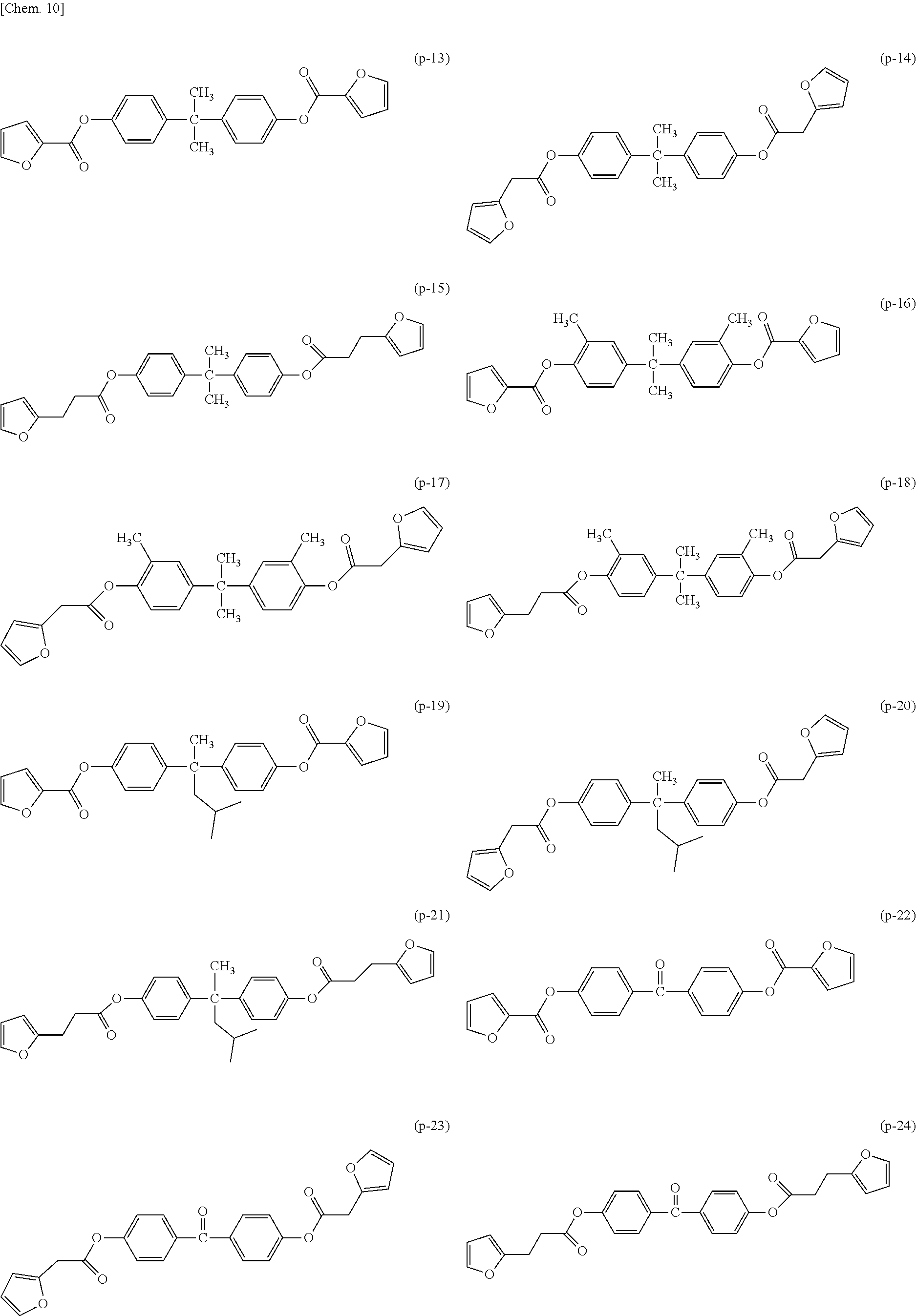

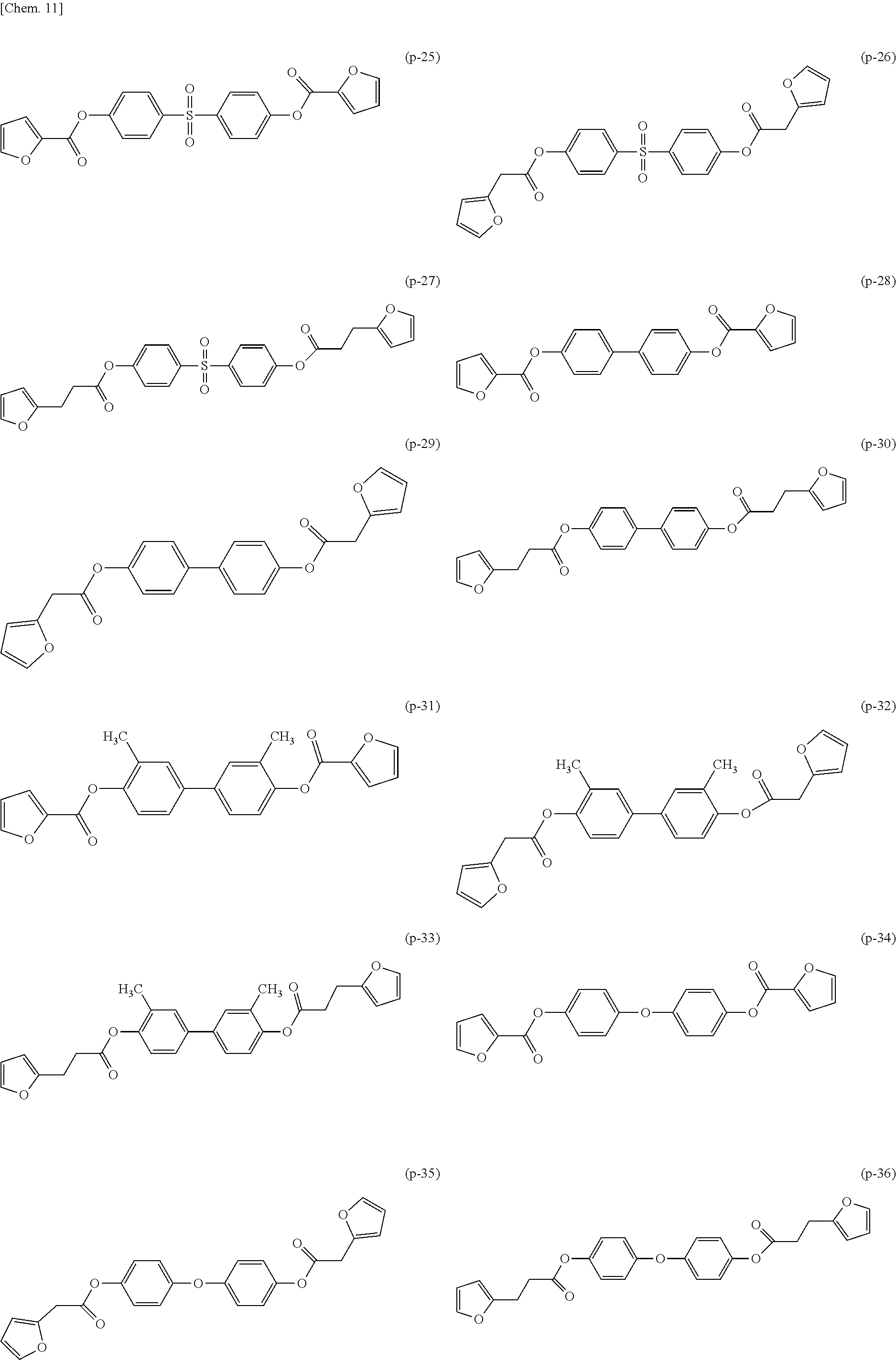

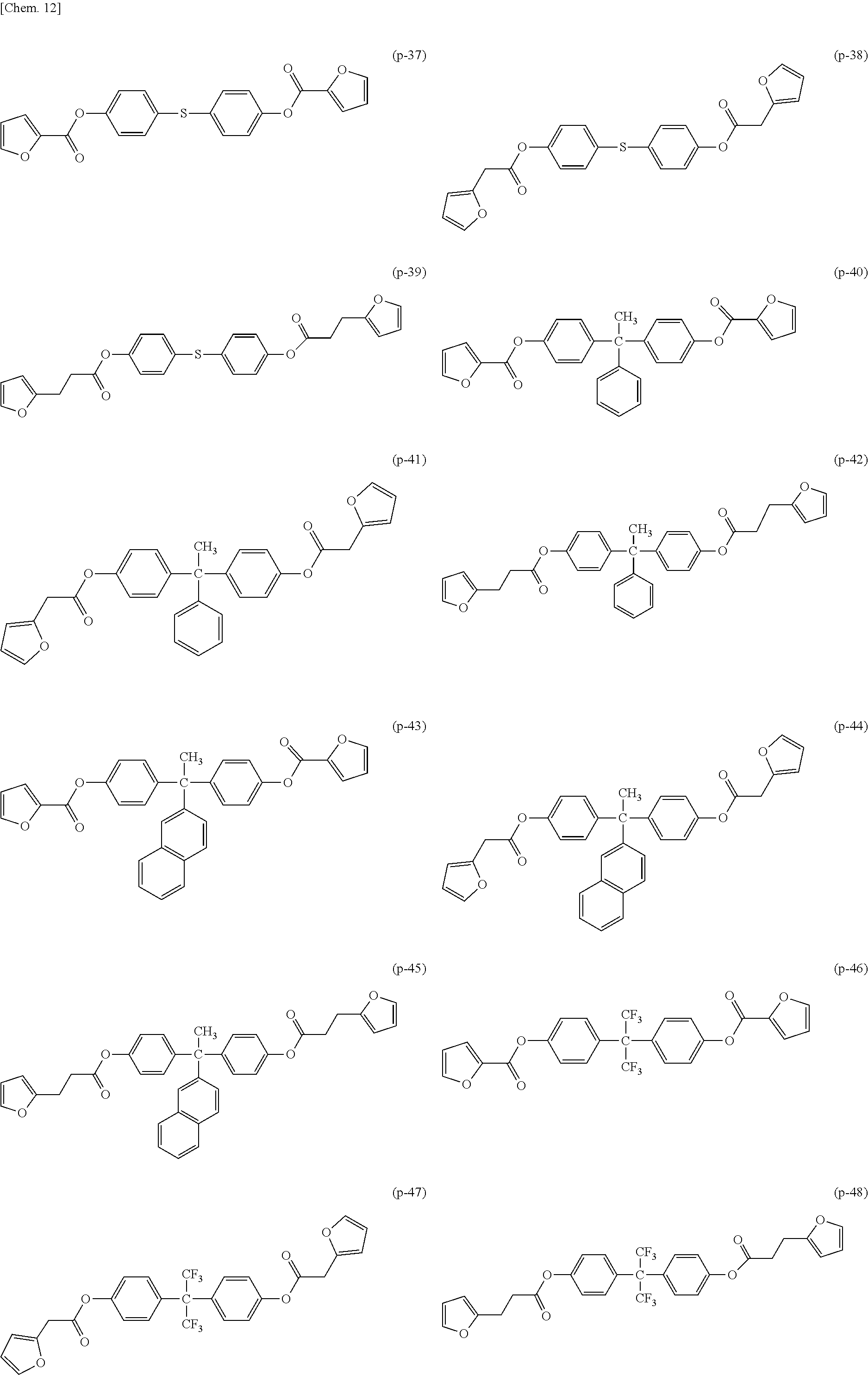

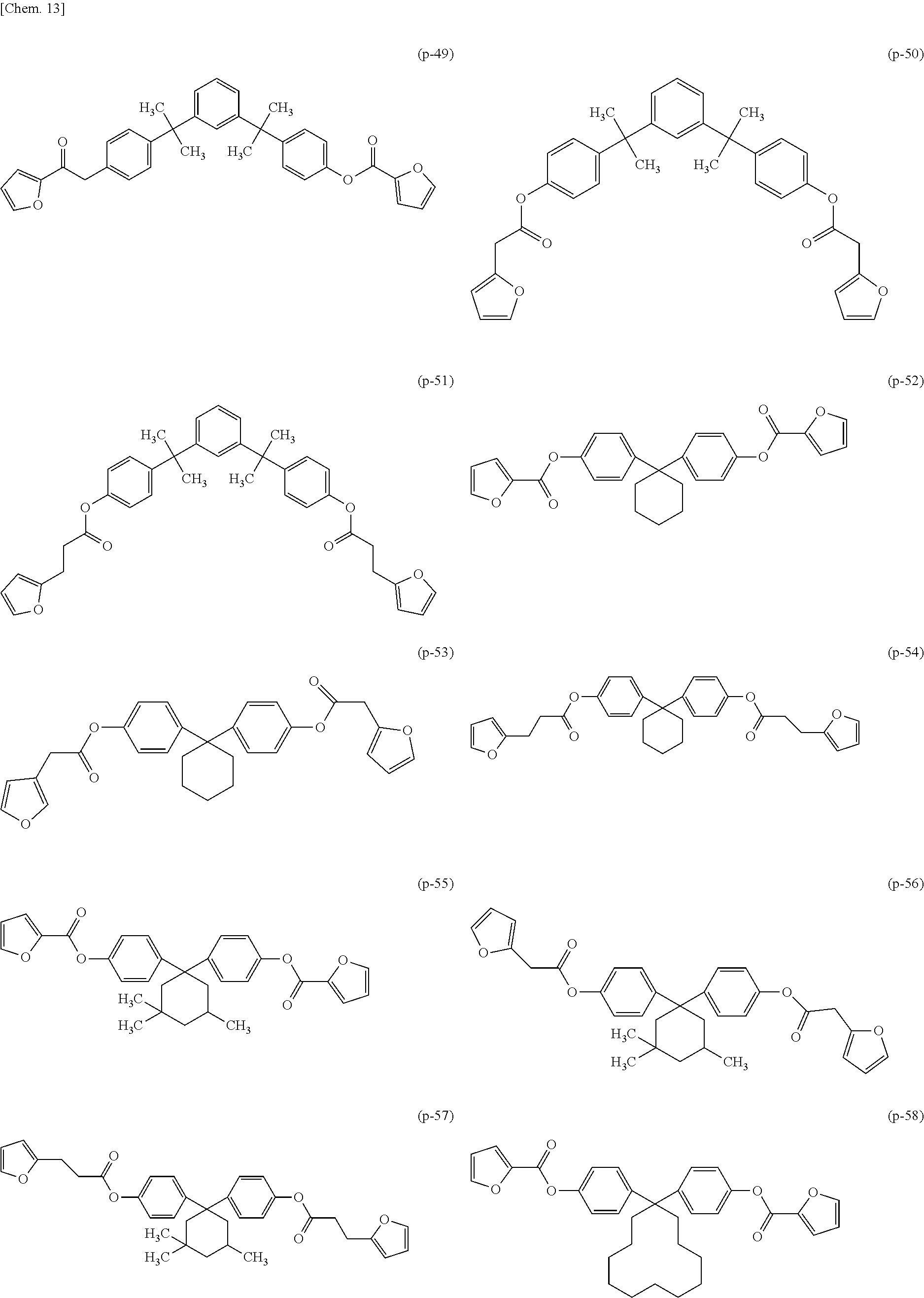

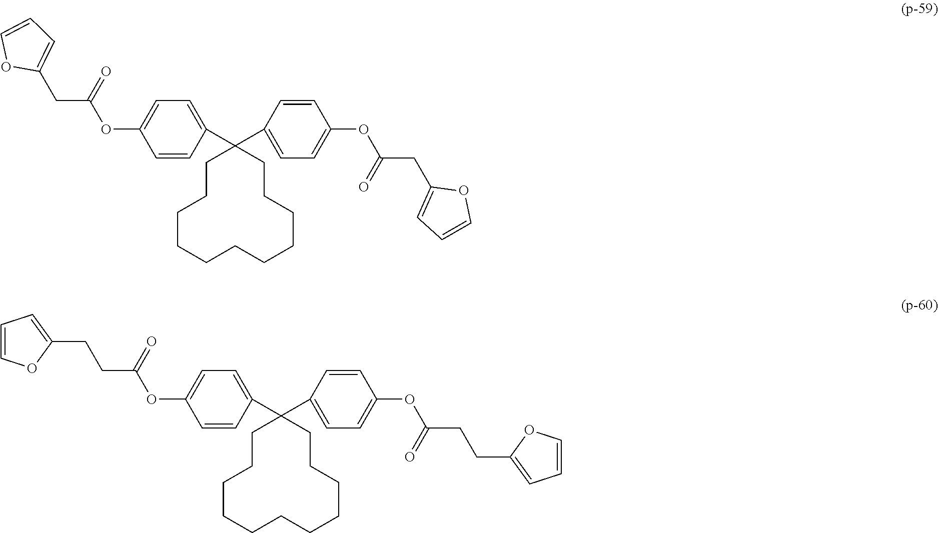

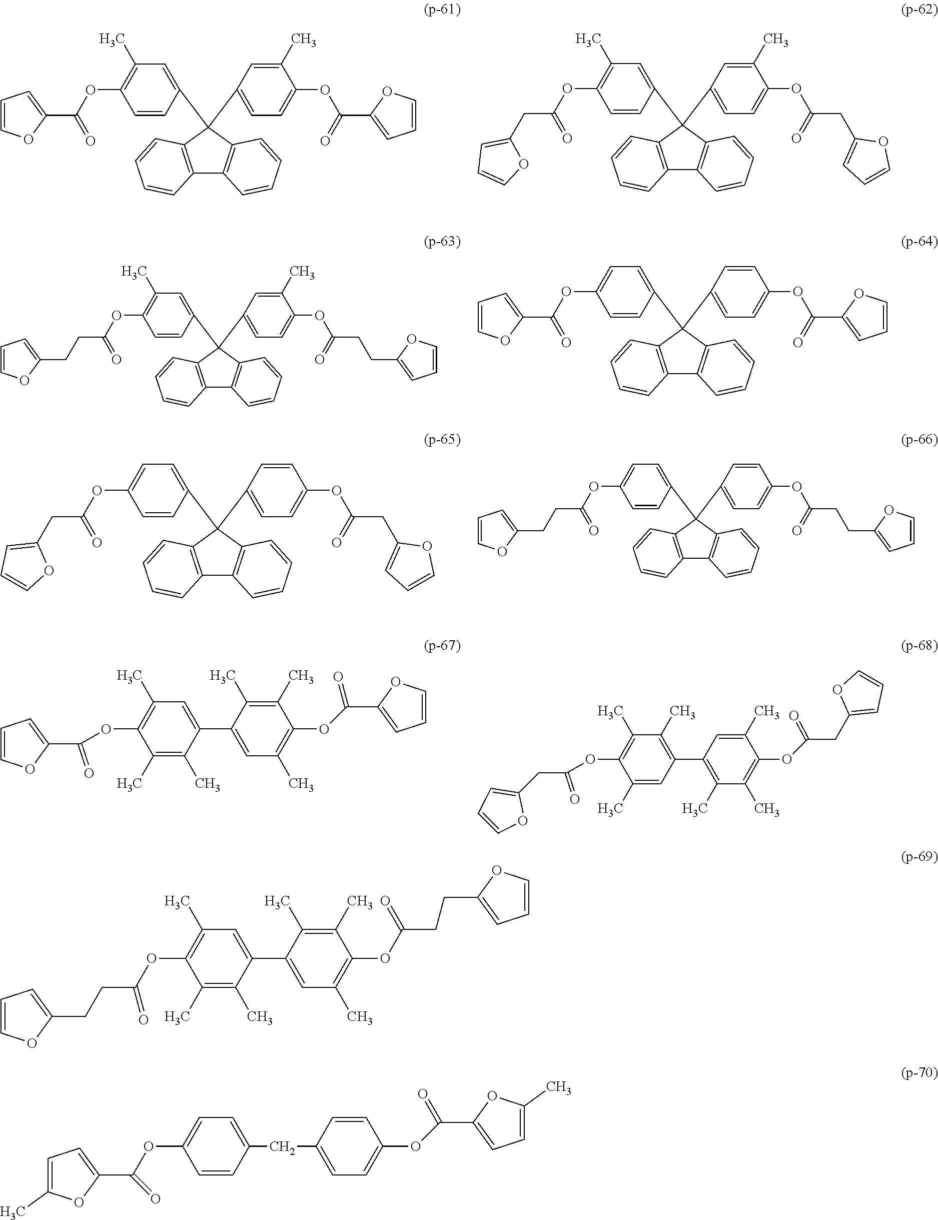

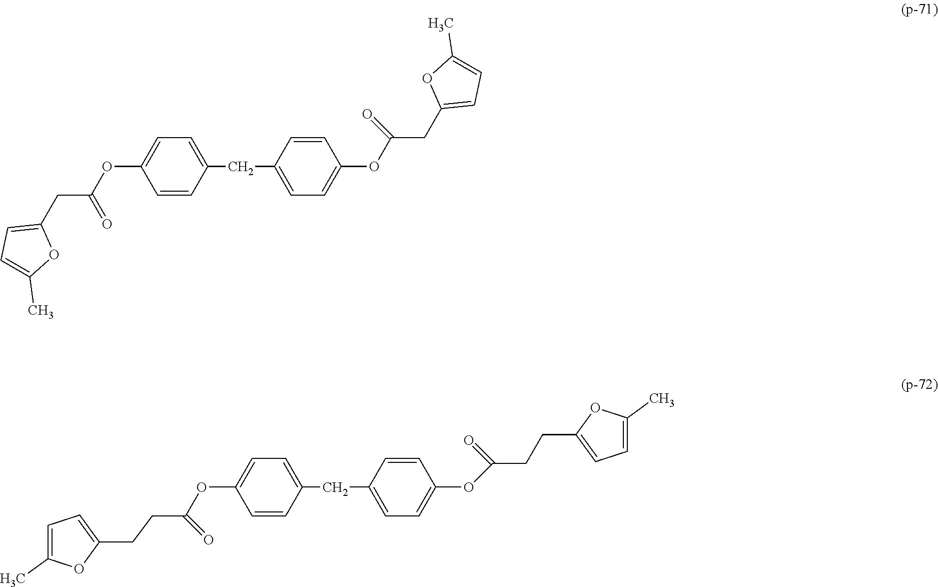

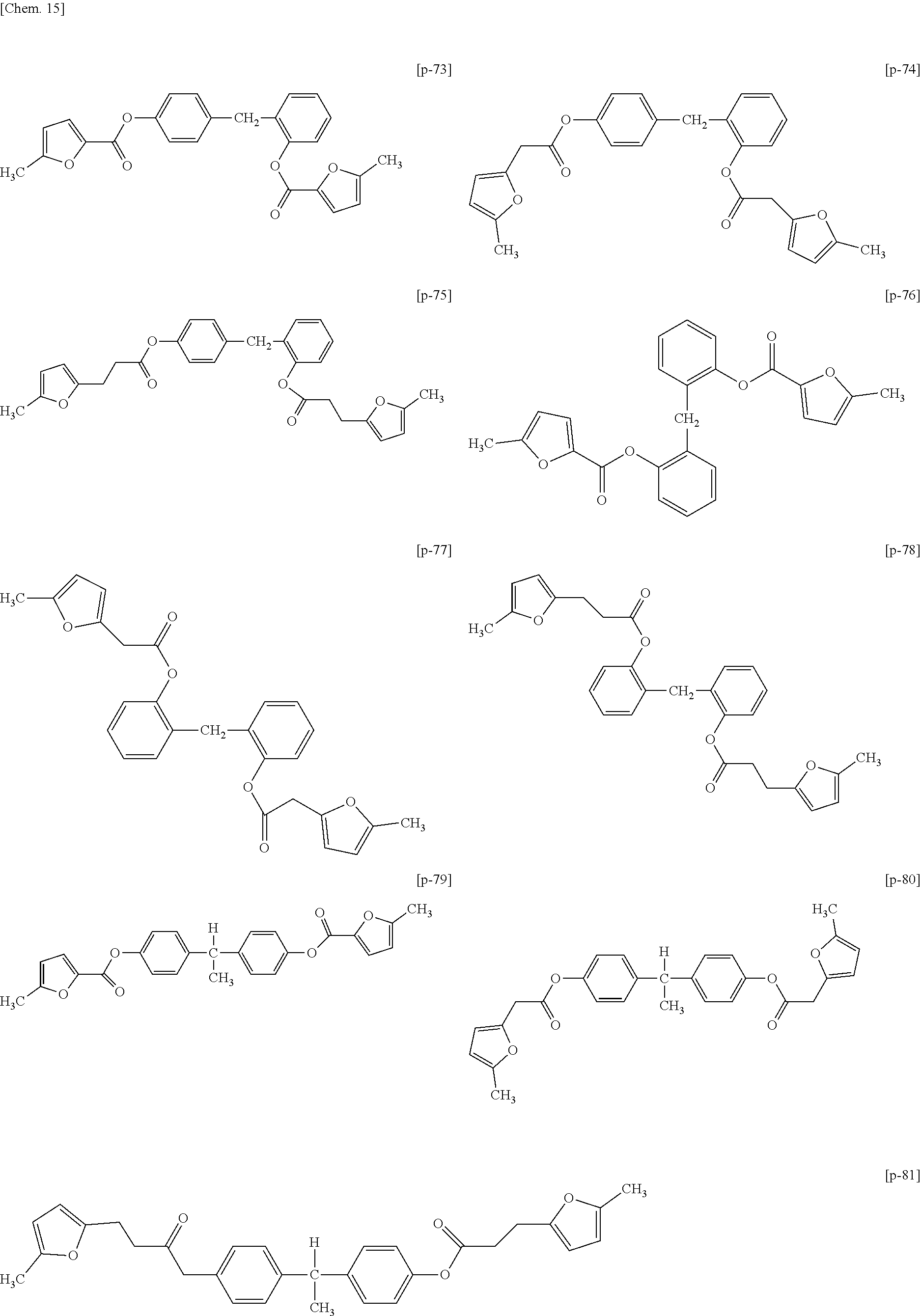

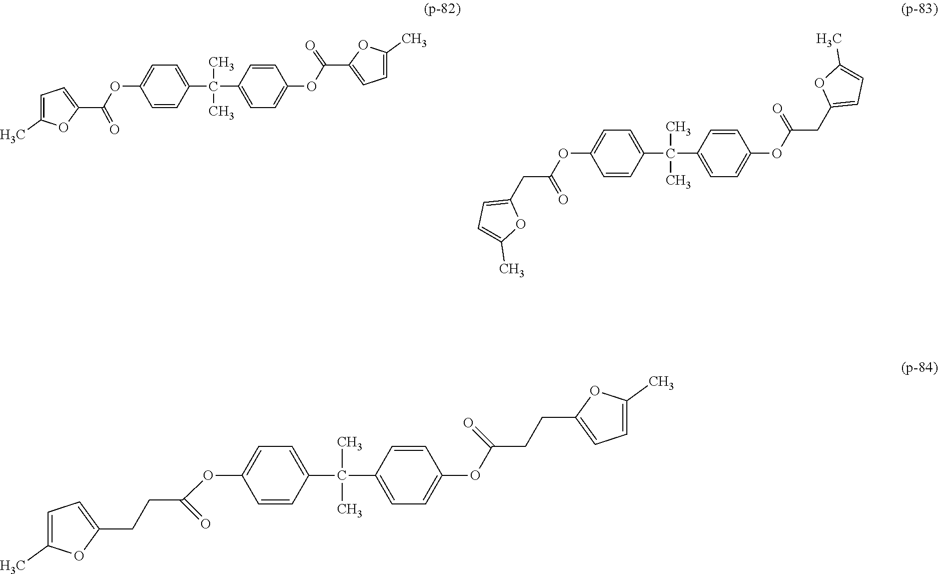

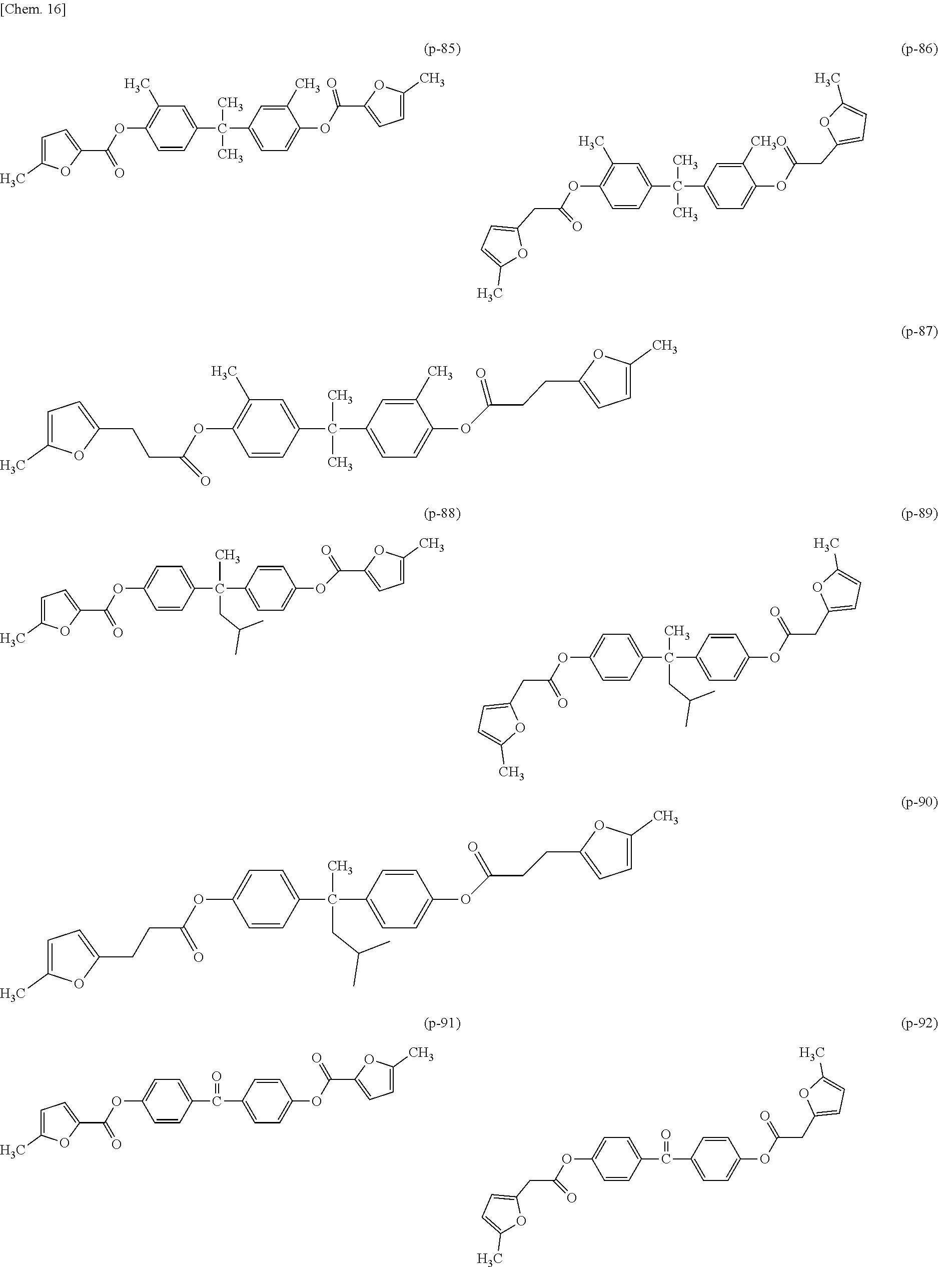

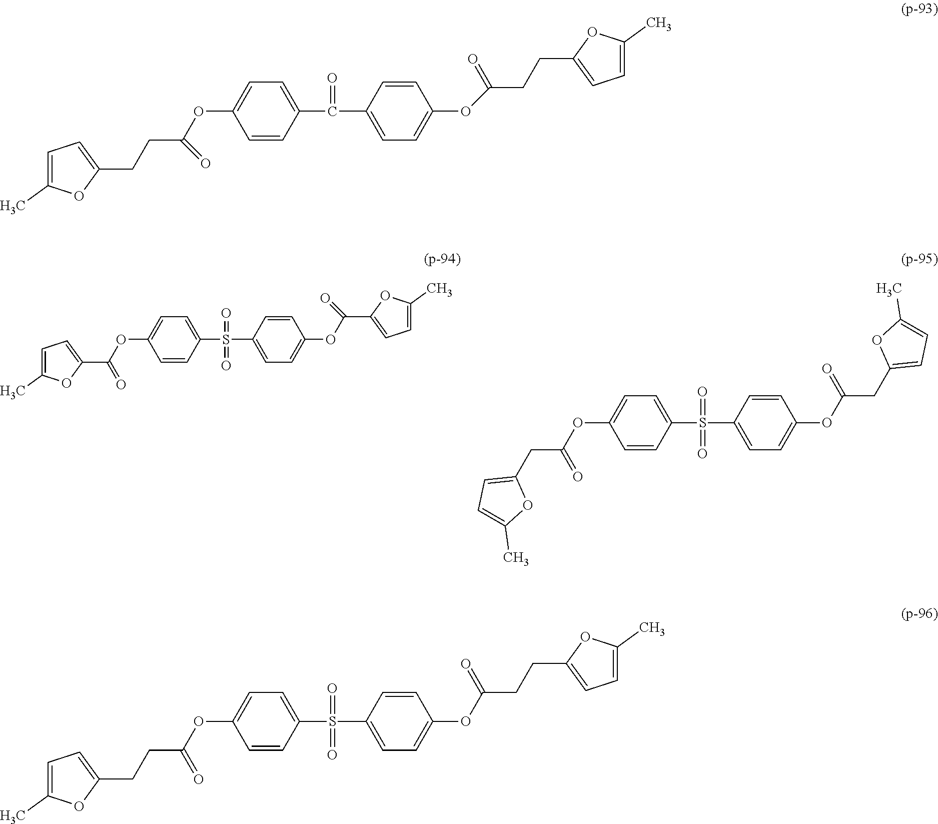

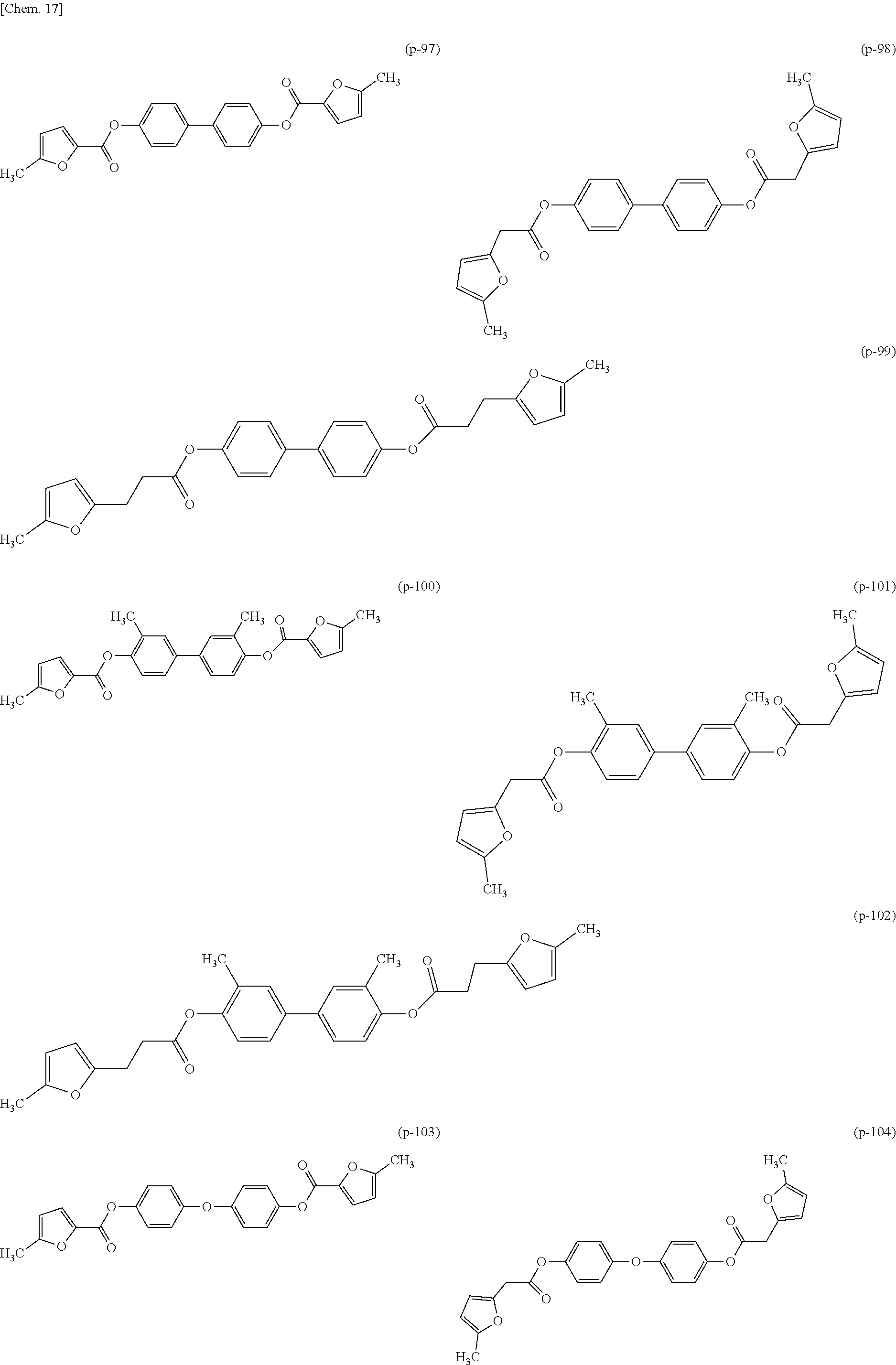

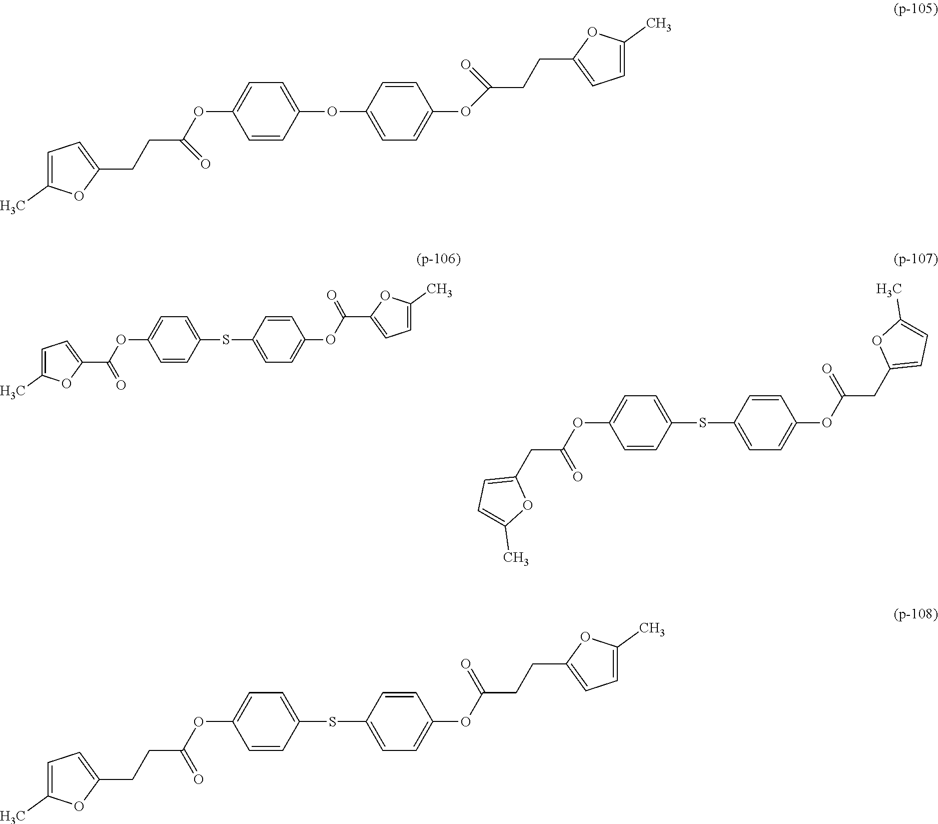

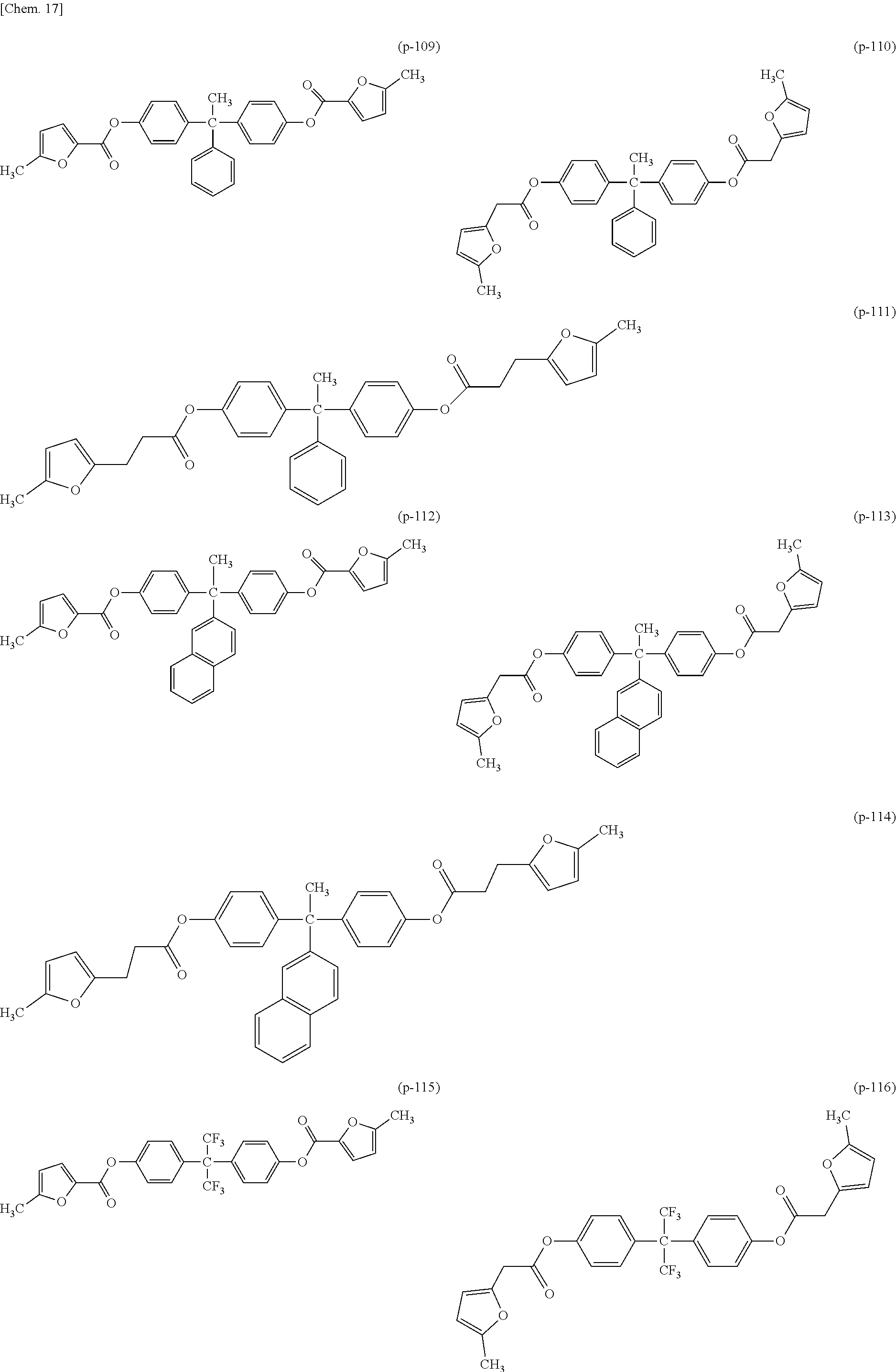

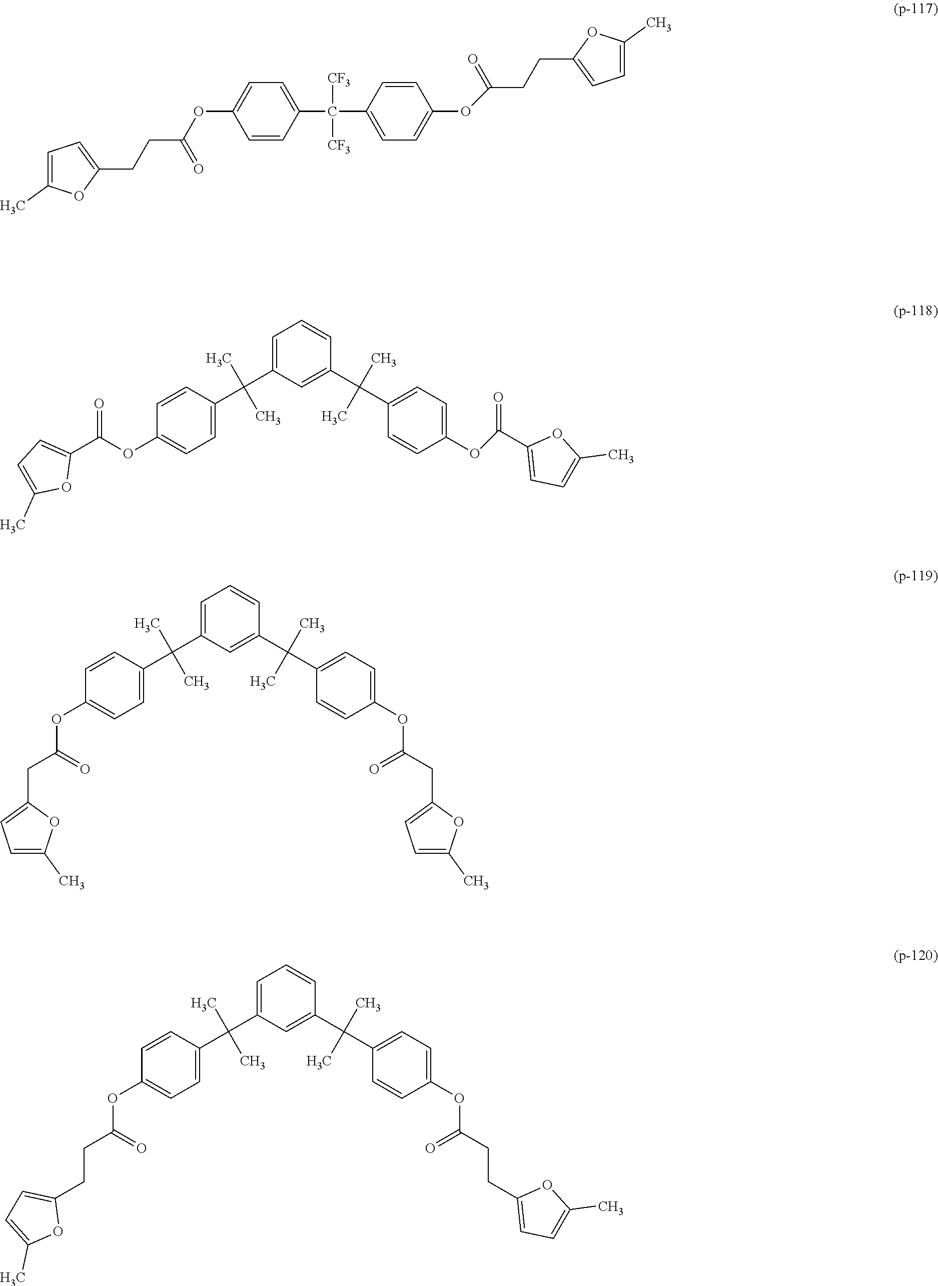

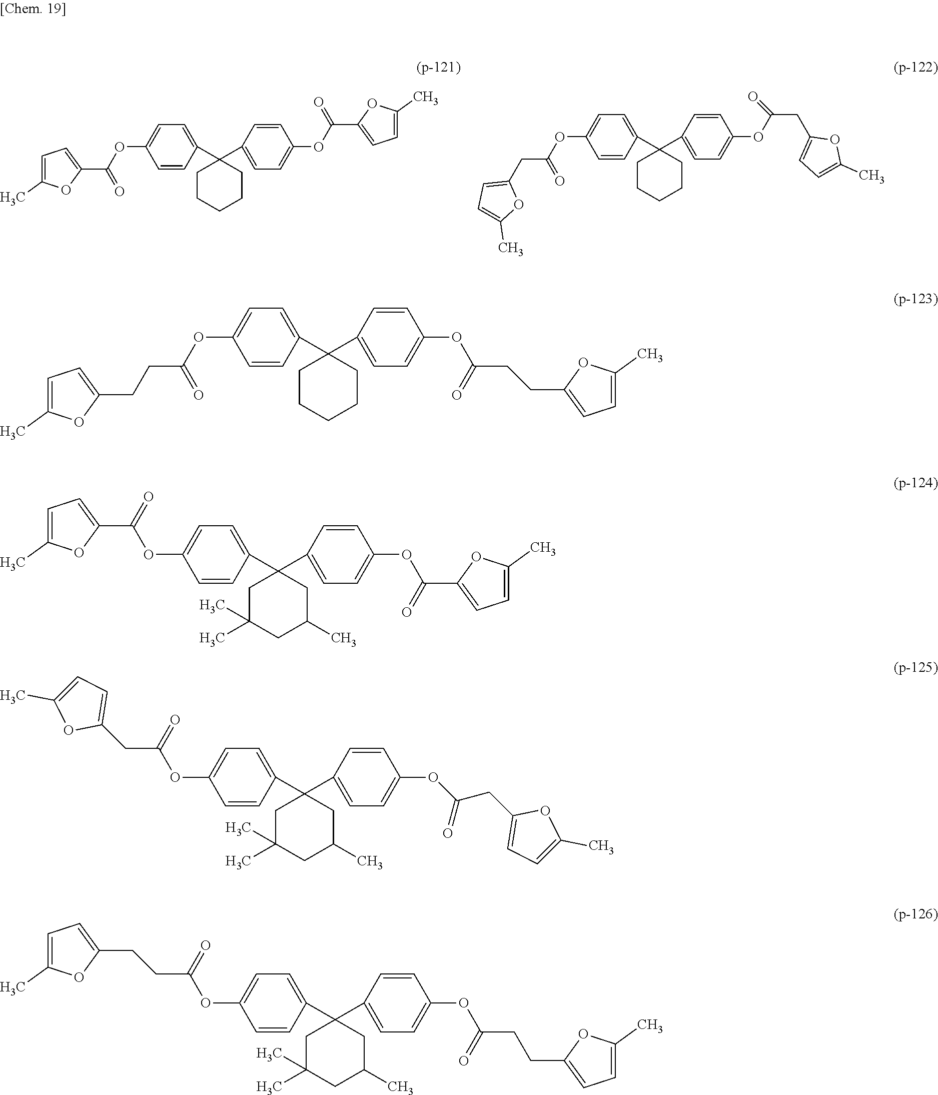

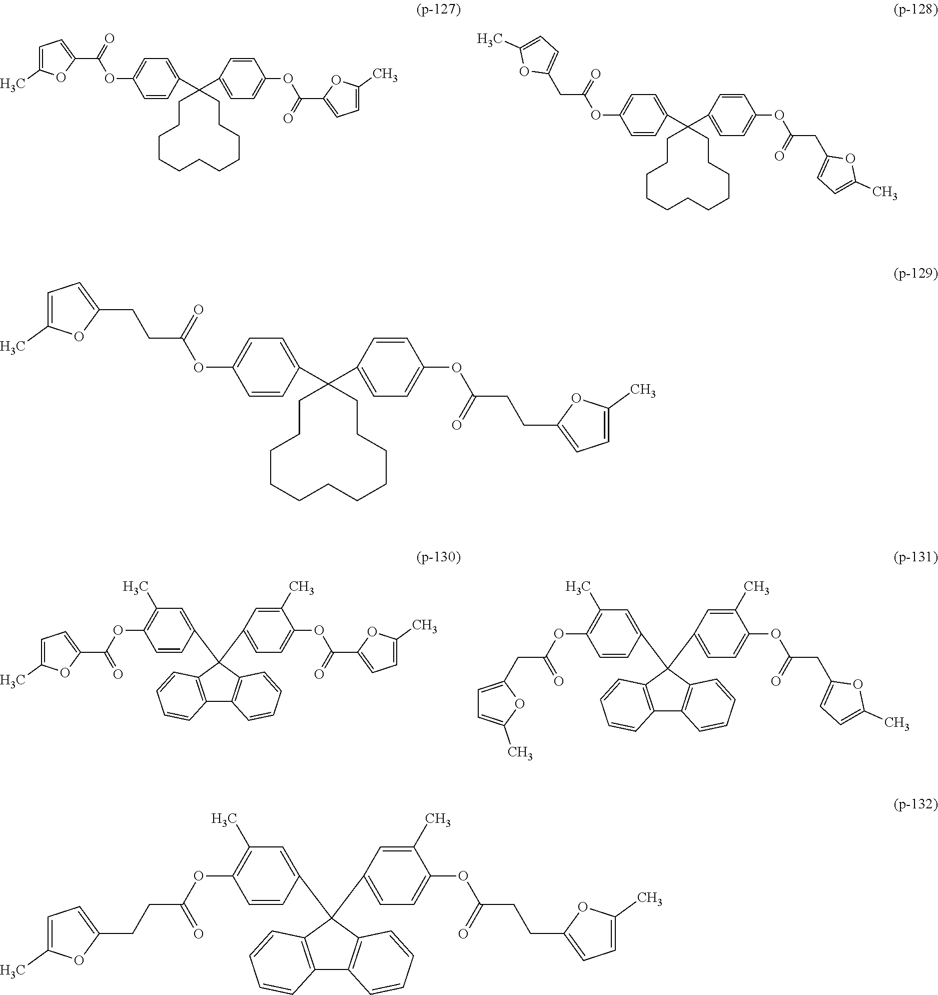

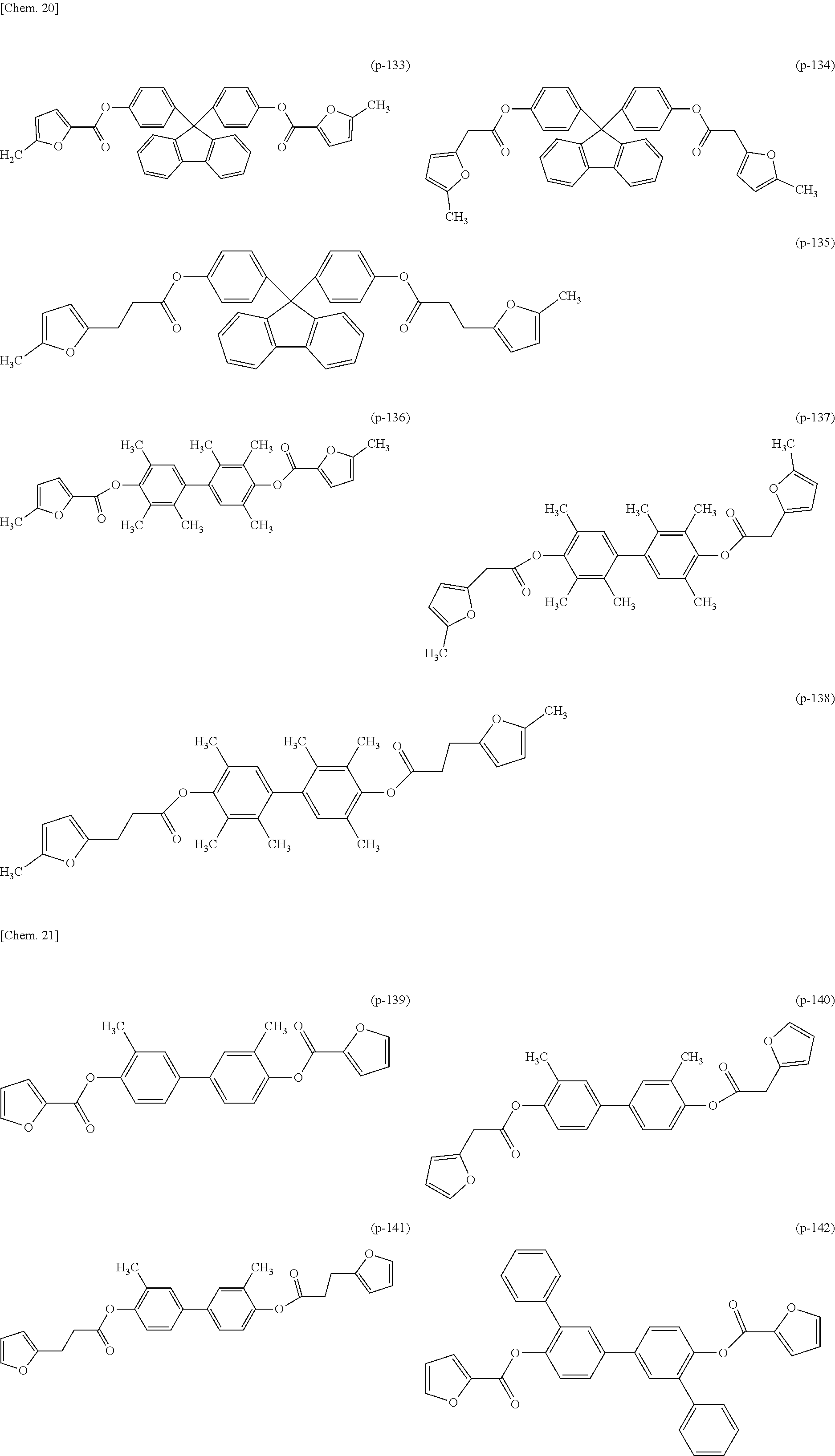

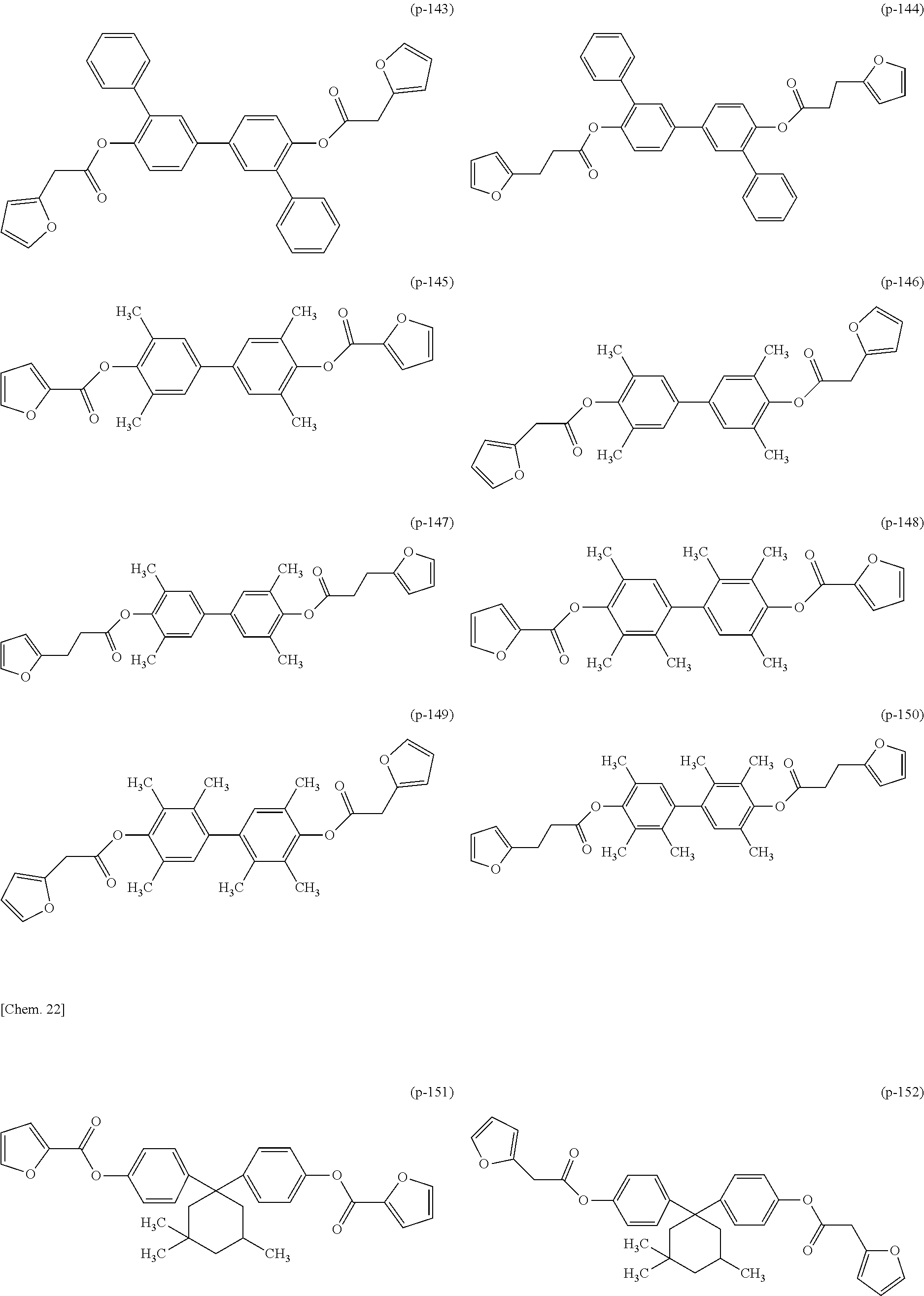

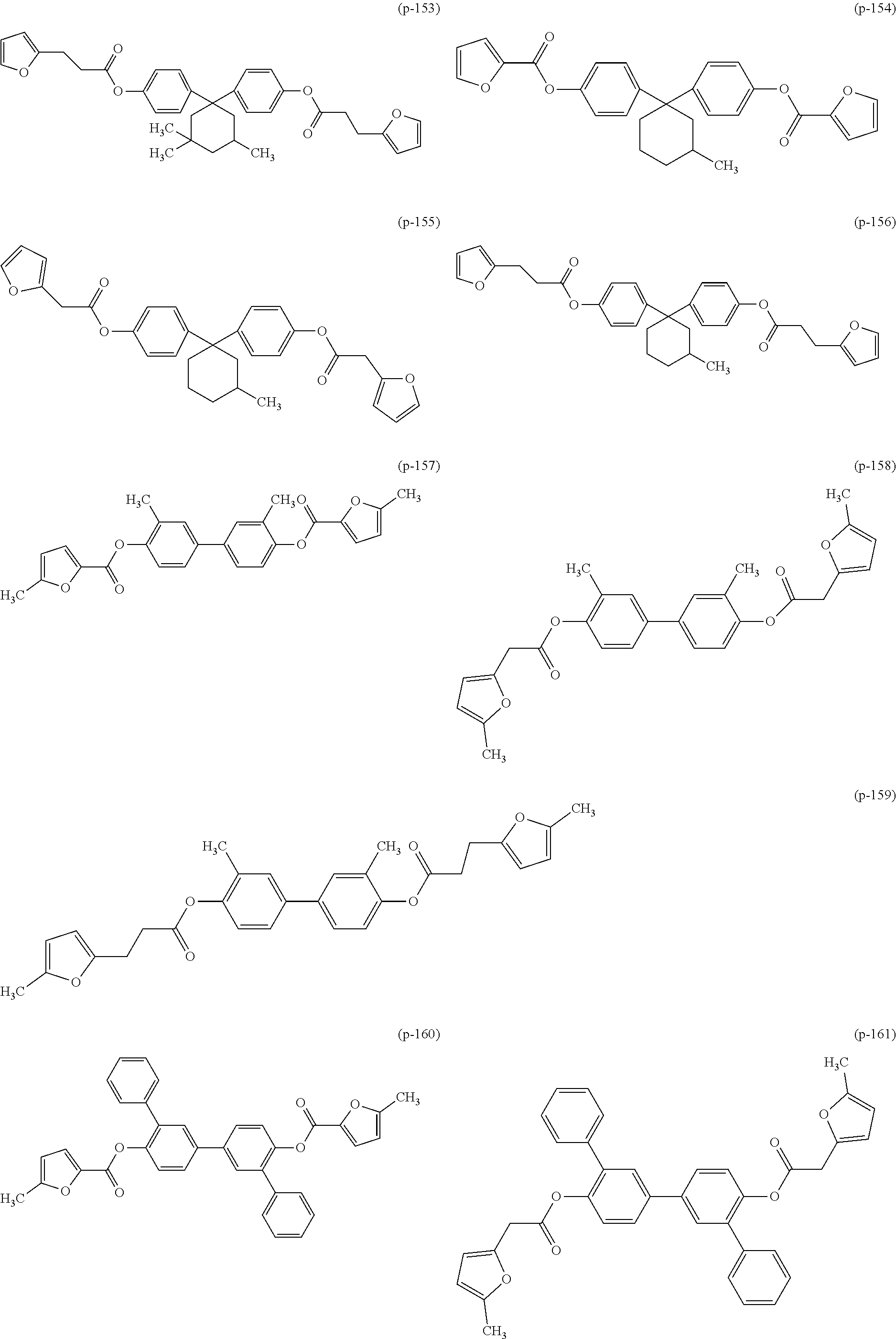

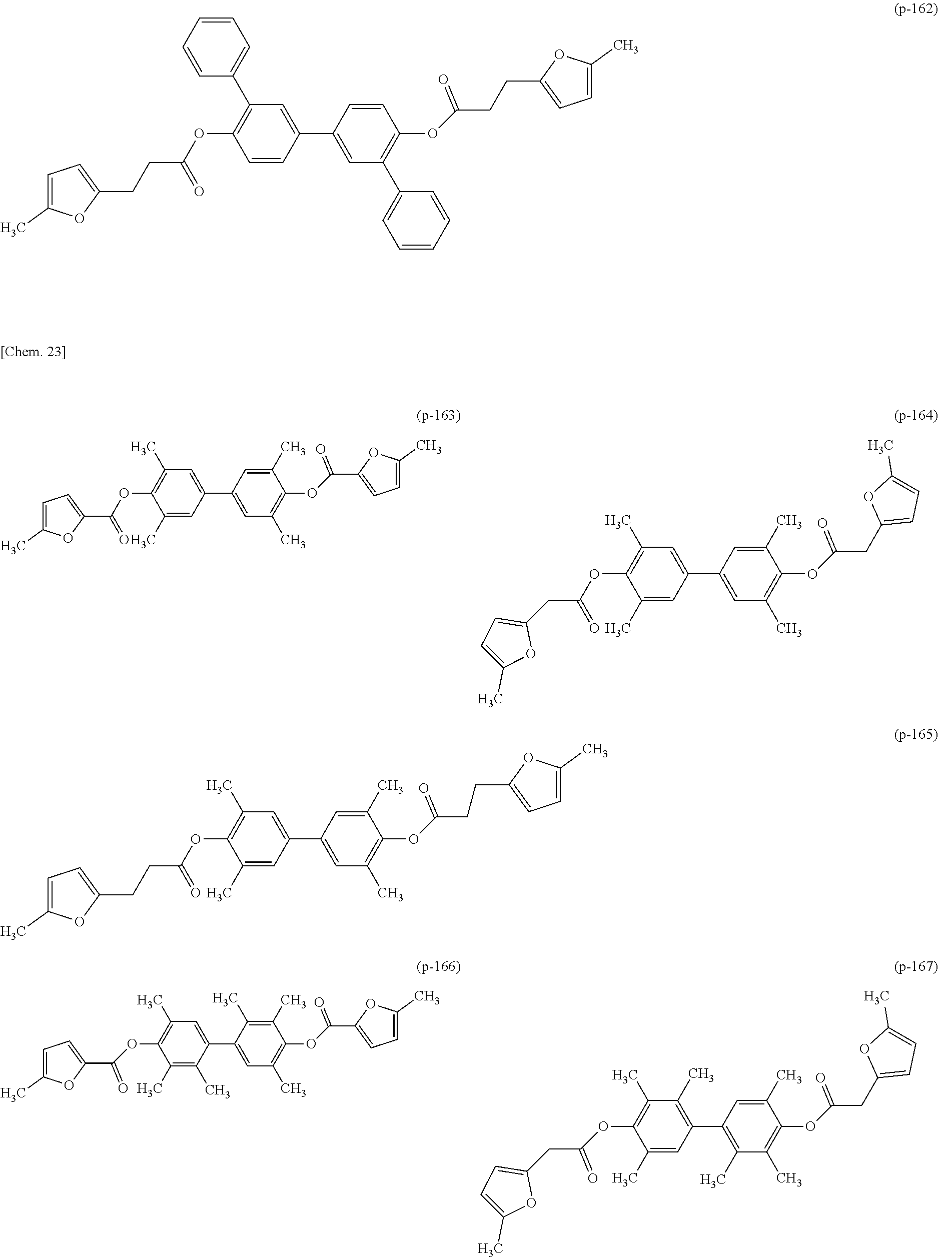

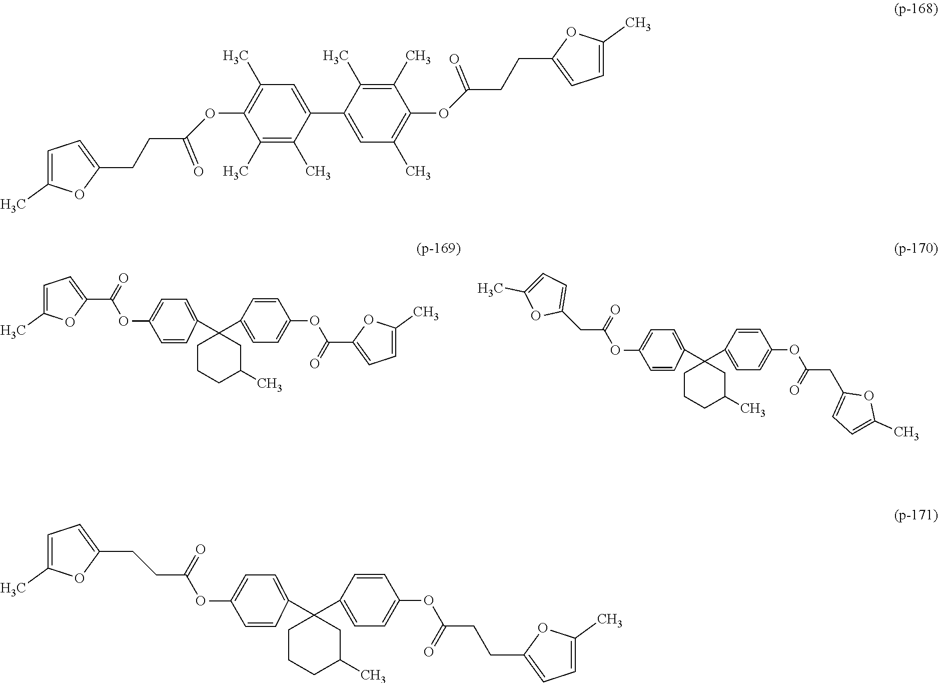

### <Method for Producing Ester Compound Represented by General Formula (1)>

For the ester compound represented by general formula (1), there are no particular limitations on the starting materials in the production of the ester compound and the method for producing the ester compound. For example, as illustrated by the following reaction formula, a production method in which an ester compound represented by general formula (2) is synthesized by an esterification step of reacting a bisphenol compound represented by general formula (5) and an acid anhydride represented by general formula (6) to synthesize the ester compound represented by general formula (2), and then an ester compound represented by general formula (1) is obtained by a transesterification reaction step of reacting the ester compound represented by general formula (2) and a furan-containing carboxylic acid represented by general formula (8) to obtain the ester compound represented by general formula (1), may be used.

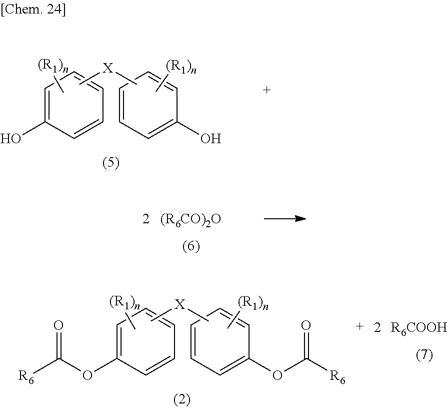

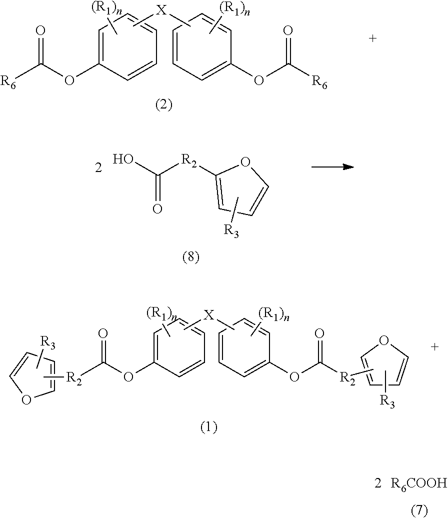

(R₁, X, and n in general formula (5) and R₂ and R₃ in general formula (8) are as defined in general formula (1), and R₆ in general formula (6) and general formula (7) are as defined in general formula (2) described later.)

### (Esterification Step)

As a method of the reaction in the esterification step in the above production method, a conventionally known esterification reaction method can be used.

Specific examples of the bisphenol compound represented by general formula (5) include bisphenol F (bis(2-hydroxyphenyl) methane, 2-hydroxyphenyl-4-hydroxyphenylmethane, bis(4-hydroxyphenyl) methane), bisphenol E (1,1-bis(4-hydroxyphenyl) ethane), bisphenol A (2,2-bis(4-hydroxyphenyl) propane), bisphenol C (2,2-bis(4-hydroxy-3-methylphenyl) propane), 2,2-bis(4-hydroxyphenyl)-4-methylpentane, 4,4′-dihydroxybiphenyl, 4,4′-dihydroxy-3,3′-dimethylbiphenyl, 4,4′-dihydroxy-3, 3′,5, 5 ‘-tetramethylbiphenyl, 4, 4’-dihydroxy-2, 2 ‘, 3, 3’, 5, 5 ‘-hexamethylbiphenyl, bis(4-hydroxyphenyl) ether, 4, 4’-dihydroxybenzophenone, bis(4-hydroxyphenyl) sulfone, bis(4-hydroxyphenyl) sulfide, 1,1-bis(4-hydroxyphenyl)-1-phenylethane, 1,1-bis(4-hydroxyphenyl)-1-naphthylethane, 2,2-bis(4-hydroxyphenyl) hexafluoropropane, bisphenol Z (1,1-bis(4-hydroxyphenyl)cyclohexane), bisphenol TMC (1,1-bis(4-hydroxyphenyl)-3,3,5-trimethylcyclohexane), 1,1-bis(4-hydroxyphenyl)-3-methylcyclohexane, 1,1-bis(4-hydroxyphenyl)-4-methylcyclohexane, 1,1-bis(4-hydroxyphenyl)cyclododecane, and 9,9-bis(4-hydroxy-3-methylphenyl) fluorene.

Specific examples of the acid anhydride represented by general formula (6) include acetic anhydride and benzoic anhydride. The definition and preferred forms of Re in general formula (6) are the same as those in general formula (2) described later.

In the above production method, the amount of the acid anhydride represented by general formula (6) used is preferably in the range of 2.0 to 10.0 mol, more preferably in the range of 2.0 to 8.0 mol, still more preferably in the range of 2.0 to 4.0 mol, relative to 1 mol of the bisphenol compound represented by general formula (5).

The reaction temperature is typically in the range of 50° C. to 150° C., preferably in the range of 80° C. to 140° C. The reaction pressure may be either normal pressure or reduced pressure.

The ester compound represented by general formula (2) can be produced through this esterification step.

### (Ester Compound Represented by General Formula (2))

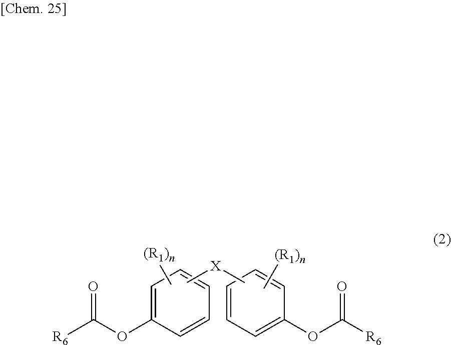

(In the formula, R₁, X, and n are as defined in general formula (1), and each R₆ independently represents a monovalent hydrocarbon group having 1 to 20 carbon atoms.)

R₁, X, and n in general formula (2) are as defined in general formula (1), and preferred forms are also the same.

Each Re in general formula (2) is preferably independently a monovalent hydrocarbon group having 1 to 10 carbon atoms, more preferably independently an alkyl group having 1 to 6 carbon atoms or a phenyl group, still more preferably independently a methyl group or a phenyl group, particularly preferably a methyl group.

Specific examples of the monovalent hydrocarbon group having 1 to 20 carbon atoms represented by R₆ in general formula (2) include chain monovalent hydrocarbon groups such as a methyl group, an ethyl group, a propyl group, an isobutyl group, a butyl group, a hexyl group, an octyl group, and a decyl group, cyclic monovalent hydrocarbon groups such as a cyclohexyl group, and monovalent aromatic hydrocarbon groups such as a phenyl group and a naphthyl group.

The ester compound represented by general formula (2) obtained through the esterification step may be used as a raw material for the transesterification reaction step described later. The ester compound may be used as it is contained in the reaction liquid of the esterification reaction, may be used after being purified by removing a carboxylic acid represented by general formula (7) produced as a result of the esterification reaction by distillation, or may be used after being purified by mixing the esterification reaction liquid with a solvent and performing a crystallization operation.

### (Transesterification Reaction Step)

As a method of the reaction in the transesterification reaction step in the above production method, a conventionally known transesterification reaction method can be used.

R₂ and R₃ in general formula (8) are as defined in general formula (1), and preferred forms and specific examples are also the same.

Specific examples of the furan-containing carboxylic acid represented by general formula (8) include 2-furancarboxylic acid, 3-furancarboxylic acid, 2-methyl-3-furancarboxylic acid, and 3-methyl-2-furancarboxylic acid.

The amount of the furan-containing carboxylic acid represented by general formula (8) used is preferably in the range of 2.0 to 10.0 mol, more preferably in the range of 2.0 to 8.0 mol, still more preferably in the range of 2.0 to 4.0 mol, relative to 1 mol of the ester compound represented by general formula (2).

It is preferable to use a base as a catalyst in the reaction between the ester compound represented by general formula (2) and the furan-containing carboxylic acid represented by general formula (8). Specific examples of such a base include organic bases such as amine bases, inorganic alkali metal compounds such as hydroxides, carbonates, and hydrogen carbonate compounds of alkali metals, and organic alkali metal compounds such as salts of alkali metals and alcohols, phenols, or organic carboxylic acids, and also include mixtures thereof, but are not limited thereto.

The reaction is typically carried out in the presence of a solvent. For reasons of, for example, ease of operation in industrial production and improvement in reaction rate, it is preferable to use a reaction solvent in the reaction. The solvent that can be used is not particularly limited as long as it does not distill out of a reaction vessel at the following reaction temperature and is inert to the transesterification reaction. Specific examples include aromatic hydrocarbon ether solvents including alkyl aryl ethers such as phenetole and butyl phenyl ether and diaryl ethers such as diphenyl ether and di-p-tolyl ether, aromatic hydrocarbon solvents such as biphenyl and terphenyl, alkyl-substituted naphthalenes such as diisopropylnaphthalene, aliphatic hydrocarbons such as decalin and kerosene, polyalkylene glycol ethers such as tetraethylene glycol dimethyl ether and diethylene glycol dibutyl ether, and organic solvents such as Therm-S series (manufactured by Nippon Steel Chemical Co., Ltd.), KSK-OIL series (manufactured by Soken Chemical & Engineering Co., Ltd.), and NeoSK-OIL series (manufactured by Soken Chemical & Engineering Co., Ltd.). The amount of the solvent used is not particularly limited as long as the reaction is not hindered, and is typically in the range of 0.5 to 20 times, preferably in the range of 1 to 10 times the amount of the ester compound represented by general formula (2) on a weight basis.

The reaction temperature is typically in the range of 40° C. to 260° C., preferably in the range of 80° C. to 255° C., more preferably in the range of 120° C. to 250° C., still more preferably in the range of 160° C. to 245° C., particularly preferably in the range of 180° C. to 240° C.

The reaction may be carried out under normal pressure conditions, or may be carried out under increased pressure or reduced pressure.

In another embodiment, a process of removing the carboxylic acid represented by general formula (7) produced during the reaction out of the system may be included. The process of removing the produced carboxylic acid represented by general formula (7) from a reaction solution is not particularly limited and can be performed by distilling the produced carboxylic acid represented by general formula (7) together with the solvent system in the reaction solution. The produced carboxylic acid represented by general formula (7) can be removed out of the reaction system by using, for example, an isobaric dropping funnel equipped with a cock, a Dimroth condenser, or a Dean-Stark apparatus.

From the final reaction mixture obtained, the ester compound represented by general formula (1) can be obtained by a known method after completion of the reaction. For example, one possible method is subjecting the reaction mixture to cooling crystallization and performing filtration after the reaction to thereby obtain the target as powder or particles. Other possible methods include adding the reaction mixture to a poor solvent to obtain the target as a precipitate, and adding a solvent to the reaction mixture to cause crystallization and performing filtration to obtain the target as powder or particles.

The ester compound represented by general formula (1) collected by any of the above-described methods can be made into a high-purity product by, for example, standard purification means such as washing with a solvent or water or recrystallization. The solvent that can be used for the crystallization and reslurrying is not particularly limited as long as it is a solvent inert to the ester compound represented by general formula (1), and specific examples include alcohol solvents such as methanol, ethanol, isopropanol, and 1-butanol, carbonyl solvents such as acetic anhydride, acetone, methyl ethyl ketone, methyl isobutyl ketone, cyclopentanone, and cyclohexanone, ether solvents such as tetrahydrofuran, methyl isobutyl ether, methyl isopropyl ether, and diphenyl ether, aromatic nonpolar solvents such as toluene, xylene, and ethylbenzene, and γ-butyrolactone, γ-valerolactone, acetonitrile, dimethylformamide, dimethylacetamide, and N-methylpyrrolidone. Among them, for example, 1-butanol, isopropanol, and diphenyl ether are preferred.

The conditions of the crystallization vary depending on the solvent used; for example, when 1-butanol is used, the amount of the solvent used is in the range of 1 part by weight to 50 parts by weight, more preferably in the range of 2 parts by weight to 30 parts by weight, particularly preferably in the range of 2 to 10 parts by weight, relative to 1 part by weight of the total amount of the composition including the ester compound represented by general formula (1) to be purified and other impurities. The temperature at the time of dissolution is in the range of 50° C. to 250° C., more preferably in the range of 70° C. to 230° C., still more preferably in the range of 80° C. to 200° C., particularly preferably in the range of 90° C. to 180° C. The cooling temperature is in the range of 0° C. to 50° C., more preferably in the range of 10° C. to 40° C., still more preferably in the range of 15° C. to 35° C. The crystallization may be performed under normal pressure conditions, or may be performed under increased pressure.

When any other solvent is used, the conditions can be appropriately changed in view of, for example, the boiling point of the solvent and the solubility of the ester compound represented by general formula (1) to be purified, other impurities, and the composition including them.

The purified product obtained through such a purification process may contain the solvent used, and thus is preferably dried by removing the solvent. One non-limiting example of the method of removing the solvent is to perform heating under normal pressure or reduced pressure to distill off the solvent.

### <Ester Compound Represented by General Formula (3)>

Among the ester compounds represented by general formula (1) used in the curable composition according to the present invention, an ester compound represented by general formula (3) is preferred because cured products obtained using the curable composition according to the present invention have more excellent heat resistance.

Among them, particularly for a secondary hydroxy group formed as a result of the reaction between an epoxy resin which is a thermosetting compound and a curing agent, the ester compound represented by general formula (3), as with the ester compound represented by general formula (1), reacts with the epoxy resin as a curing agent and esterifies the secondary hydroxy group, whereby a cured product that is less polarized and that has excellent dielectric properties is provided. Hence, the use of the ester compound represented by general formula (3) as a curing agent for an epoxy resin, as with the ester compound represented by general formula (1), is also preferred because a cured product having the above features is provided.

The ester compound represented by general formula (3), as with the ester compound represented by general formula (1), has a furan ring and thus can undergo a curing reaction also with the compound having a radical polymerizable substituent to provide a cured product.

The ester compound represented by general formula (3) has excellent solubility in solvents such as methyl ethyl ketone, cyclohexanone, N-methylpyrrolidone, toluene, propylene glycol monomethyl ether acetate, and ethyl acetate, particularly, methyl ethyl ketone, which is a solvent widely used in producing electronic components such as semiconductors, and thus is excellent in handleability.

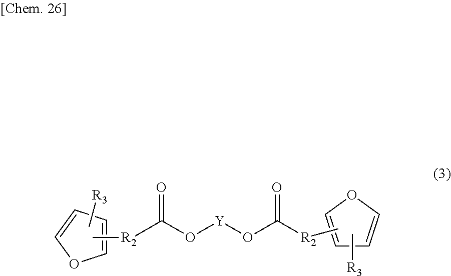

(In the formula, each R₂ independently represents a single bond or a divalent hydrocarbon group having 1 to 10 carbon atoms, each R₃ independently represents a hydrogen atom or an alkyl group having 1 to 6 carbon atoms, and Y represents a divalent group represented by general formula (3a) or general formula (3b).)

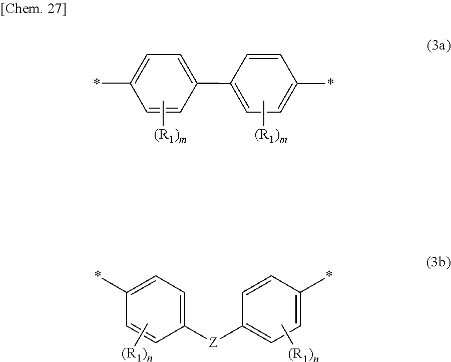

(In general formulas (3a) and (3b), each R₁ independently represents an alkyl group having 1 to 6 carbon atoms or an aryl group having 6 to 12 carbon atoms, and each * represents a bonding position. In general formula (3a), each m independently represents an integer of 1 to 4. In general formula (3b), each n independently represents 0 or an integer of 1 to 4, and Z represents a cycloalkylidene group having 7 to 20 carbon atoms.)

Specific examples and preferred forms of R₂ and R₃ in general formula (3) are the same as those of R₂ and R₃ in general formula (1). That is, each R₂ in general formula (3) is independently a single bond or a divalent hydrocarbon group having 1 to 10 carbon atoms. In the case of a divalent hydrocarbon group, specific examples include linear or branched alkylene groups having 1 to 10 carbon atoms or alkylene groups including a cyclic alkane, such as a methylene group, an ethylene group, a propane-1,2-diyl group, a propane-1,3-diyl group, a butane-1,4-diyl group, a pentane-1,5-diyl group, a hexane-1,6-diyl group, a cyclohexane-1,3-diyl group, and a cyclohexane-1,4-diyl group, alkylidene groups having 1 to 10 carbon atoms, such as an ethylidene group, a propylidene group, an isopropylidene group, a butylidene group, a cyclopentylidene group, and a cyclohexylidene group, and divalent groups including a benzene ring and having 1 to 10 carbon atoms, such as a phenylene group and groups represented by the following formulas.

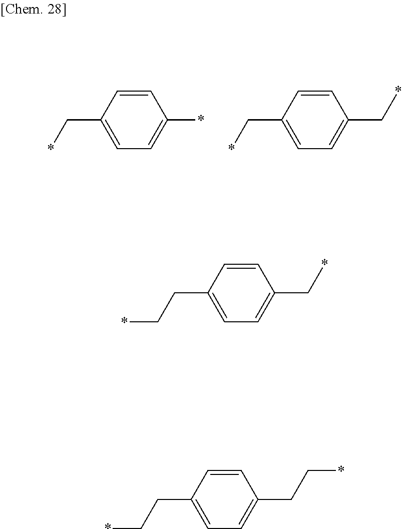

(In the formulas, * represents a bonding position.)

Of these, each R₂ is preferably independently a single bond, a linear or branched alkylene group having 1 to 10 carbon atoms, an alkylene group including a cyclic alkane, or an alkylidene group having 1 to 10 carbon atoms, more preferably independently a single bond, a linear or branched alkylene groups having 1 to 10 carbon atoms, or an alkylene group including a cyclic alkane, still more preferably independently a single bond, a linear or branched alkylene group having 1 to 6 carbon atoms, or an alkylene group including a cyclic alkane, particularly preferably a single bond. The ester compound represented by general formula (3) where each R₂ is a single bond is obtained from a biomass-derived material, and thus is suitable also because a curable composition with an improved biomass content is provided.

Each R₃ in general formula (3) is preferably independently a hydrogen atom or an alkyl group having 1 to 4 carbon atoms, more preferably independently a hydrogen atom or a methyl group, particularly preferably a hydrogen atom.

Preferred forms of R₁ in the case where Y in general formula (3) is represented by general formula (3a) are the same as those of R₁ in general formula (1). That is, each R₁ in general formula (3a) represented by Y in general formula (3) is preferably independently an alkyl group having 1 to 4 carbon atoms or a phenyl group, more preferably independently a methyl group or a phenyl group, particularly preferably a methyl group.

Each m in the case where Y in general formula (3) is represented by general formula (3a) independently represents an integer of 1 to 4, and each m is preferably independently 1 to 3, more preferably independently 2 or 3, particularly preferably 3. R₁ is preferably bonded preferentially at the ortho position with respect to the oxygen atom bonded to each benzene ring.

A more preferred form in the case where Y in general formula (3) is represented by general formula (3a) is preferably a structure selected from general formulas (3a-1) to (3a-4), more preferably a structure selected from general formulas (3a-2) to (3a-4), still more preferably general formula (3a-3) or (3a-4), particularly preferably general formula (3a-4).

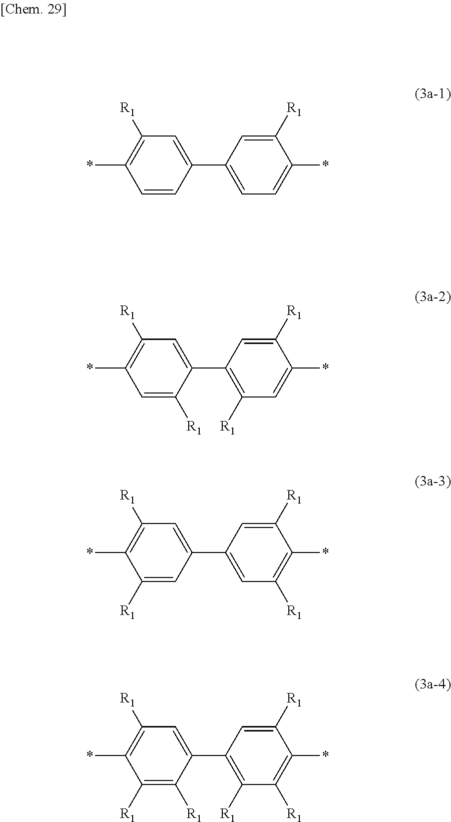

(In the formulas, each R₁ independently represents an alkyl group having 1 to 6 carbon atoms or an aryl group having 6 to 12 carbon atoms, and * represents a bonding position.)

Preferred forms of R₁ in the case where Y in general formula (3) is represented by general formula (3b) are the same as the preferred forms of R₁ in general formula (1). That is, each R₁ in general formula (3b) represented by Y in general formula (3) is preferably independently an alkyl group having 1 to 4 carbon atoms or a phenyl group, more preferably independently a methyl group or a phenyl group, particularly preferably a methyl group.

n in the case where Y in general formula (3) is represented by general formula (3b) is preferably 0, 1, or 2, more preferably 0 or 1, particularly preferably 0. The position of substitution of R₁ is preferably the ortho position with respect to each oxygen atom.

Z in general formula (3b) represents a cycloalkylidene group having 7 to 20 carbon atoms and may have a branched-chain alkyl group. This cycloalkylidene group preferably has 7 to 15 carbon atoms, more preferably has 7 to 12 carbon atoms, and particularly preferably has 7 to 9 carbon atoms. Specific examples of such a cycloalkylidene group include a 3-methylcyclohexylidene group (7 carbon atoms), a 4-methylcyclohexylidene group (7 carbon atoms), a 3,3,5-trimethylcyclohexylidene group (9 carbon atoms), and a cyclododecanylidene group (12 carbon atoms). The cycloalkylidene group is preferably a 3-methylcyclohexylidene group (7 carbon atoms), a 4-methylcyclohexylidene group (7 carbon atoms), or a 3,3,5-trimethylcyclohexylidene group (9 carbon atoms), more preferably a 3-methylcyclohexylidene group (7 carbon atoms) or a 3,3,5-trimethylcyclohexylidene group (9 carbon atoms), particularly preferably a 3,3,5-trimethylcyclohexylidene group (9 carbon atoms).

As specific examples of the ester compound represented by general formula (3), compounds (p-139) to (p-177) having the following chemical structures are shown.

The ester compound represented by general formula (3) is preferably one compound selected from compounds (p-139) to (p-177), among which compound (p-139), (p-142), (p-145), (p-148), (p-151), (p-154), or (p-172) is particularly preferred.

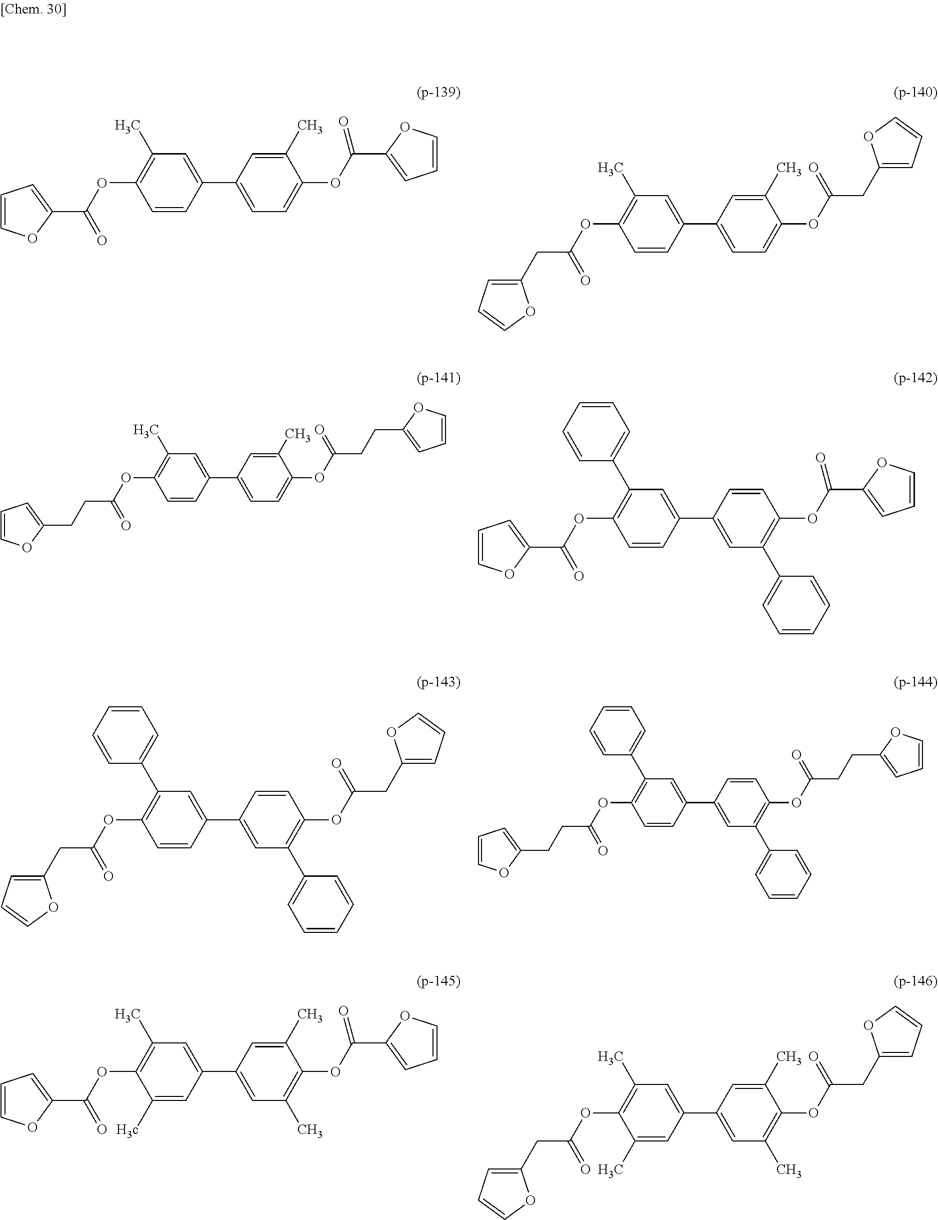

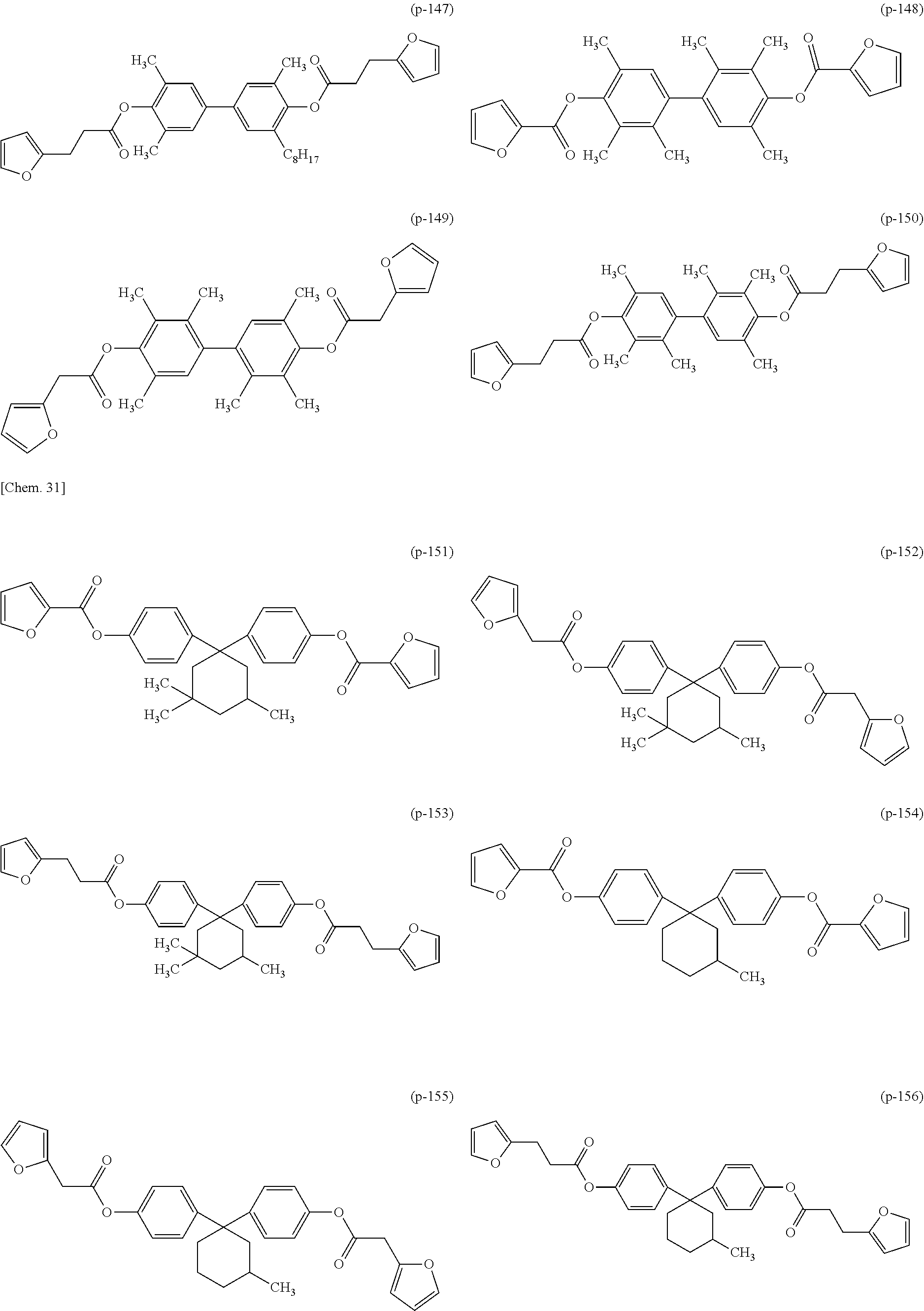

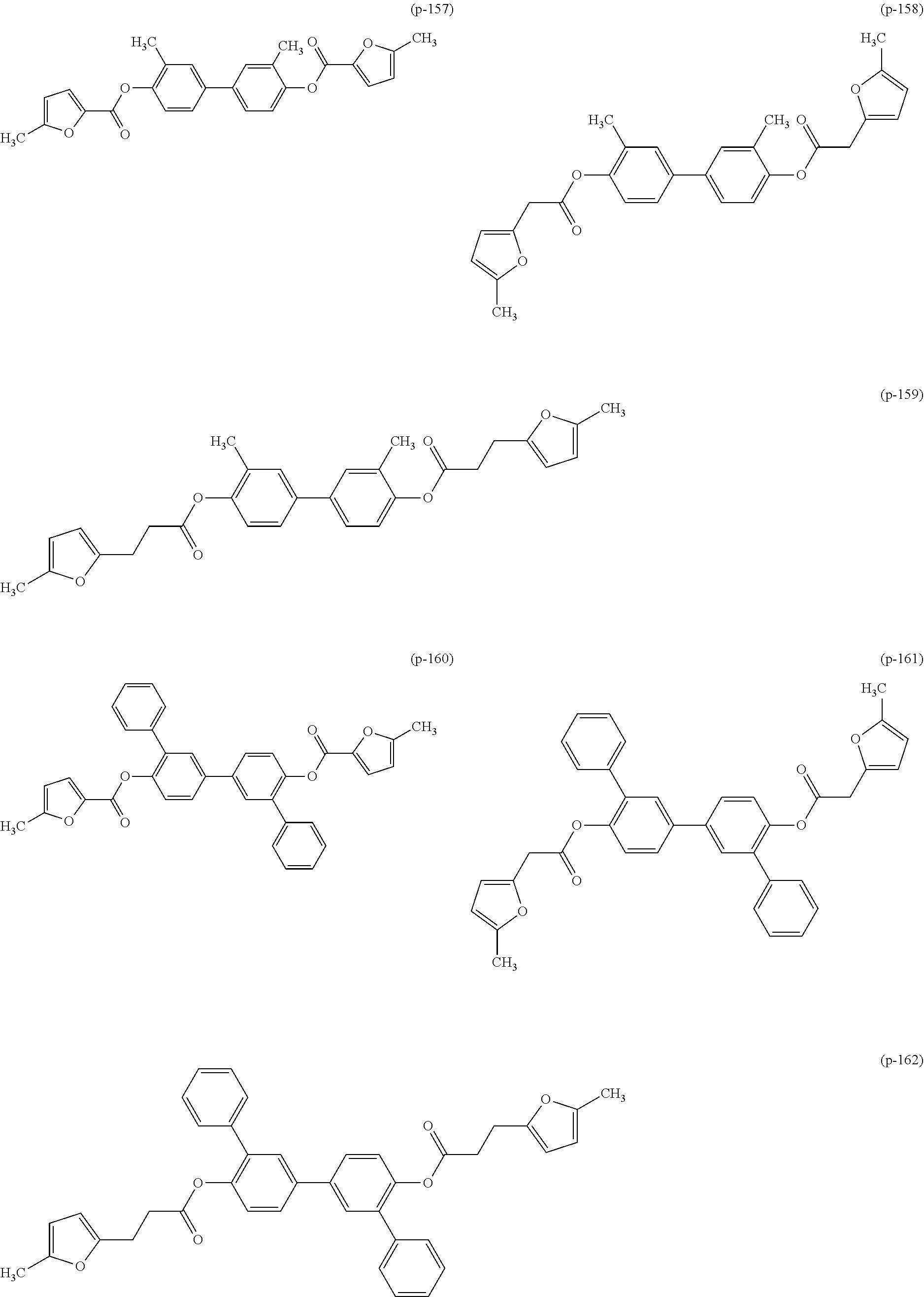

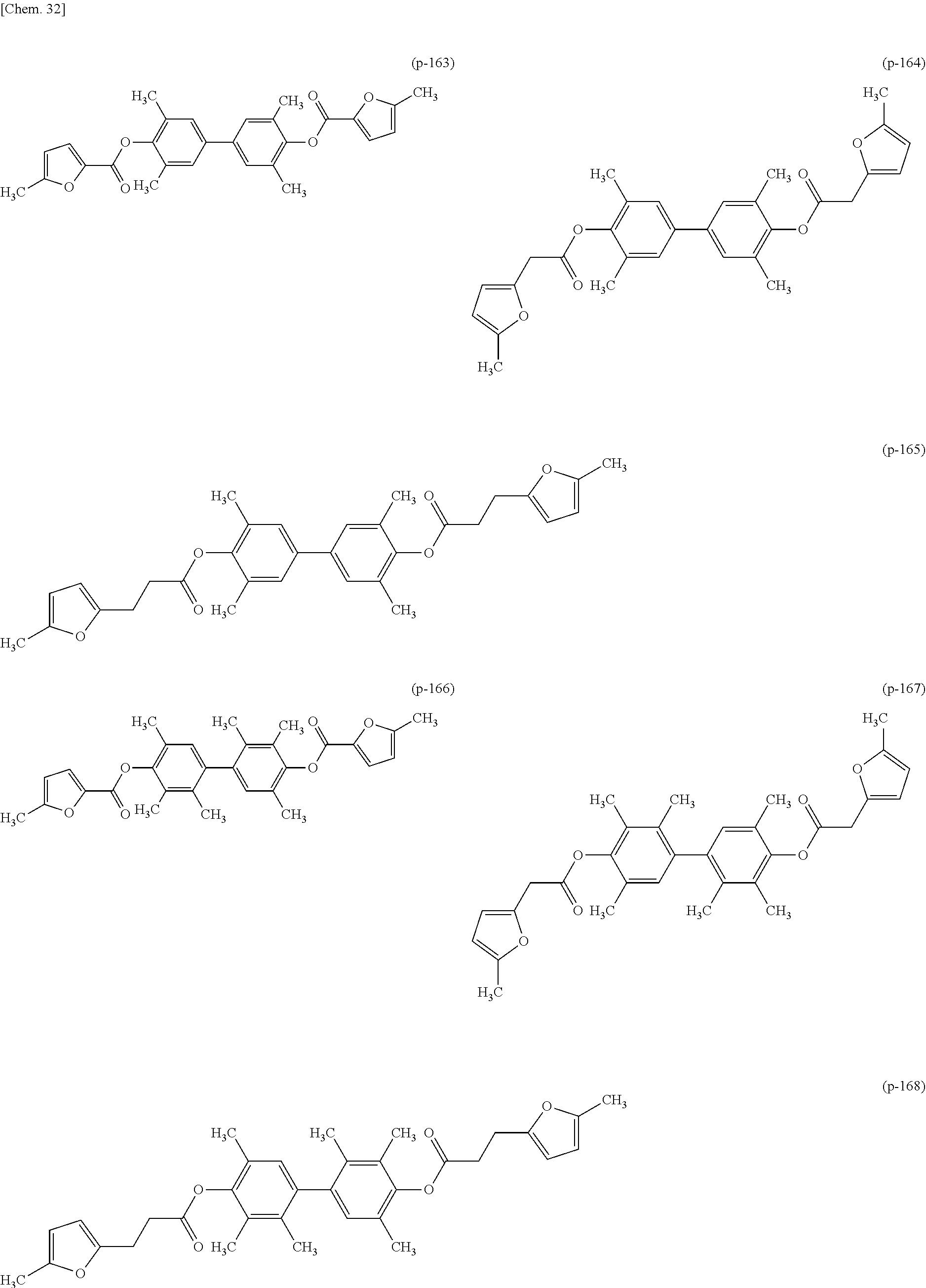

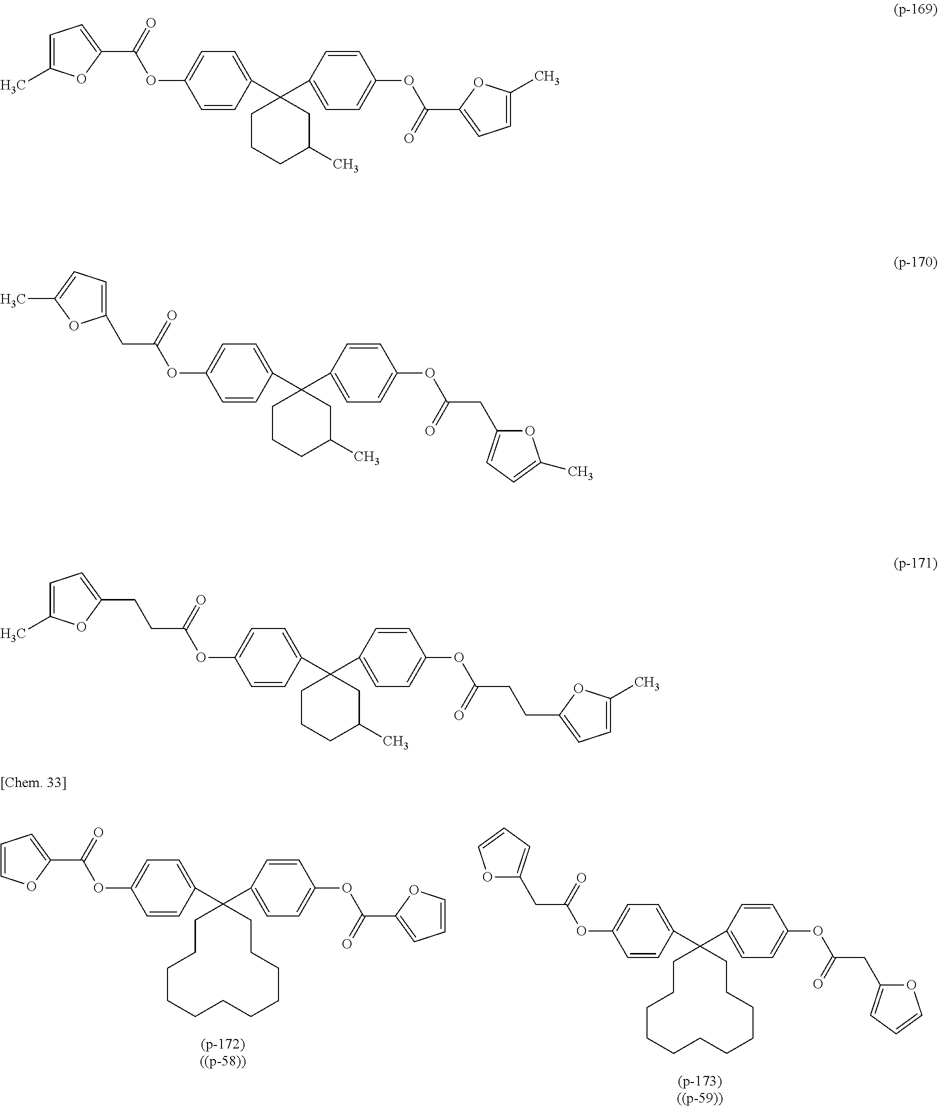

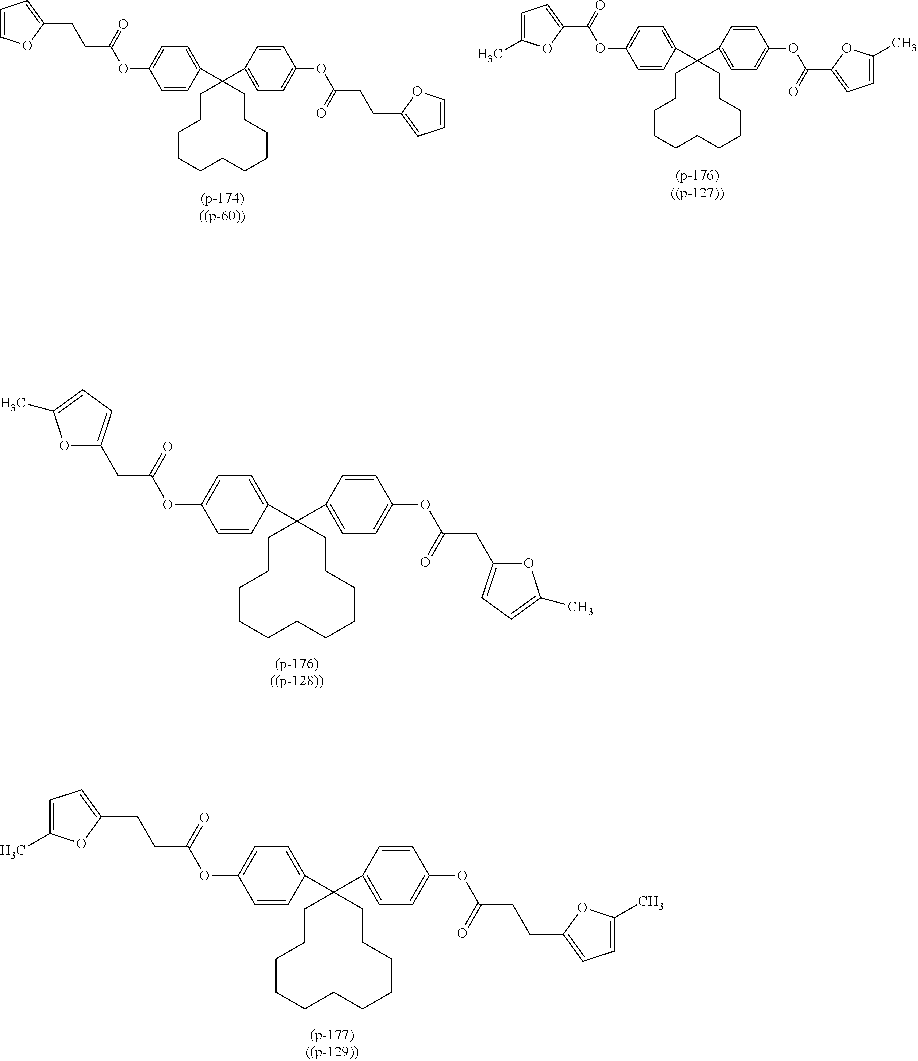

### <Method for Producing Ester Compound Represented by General Formula (3)>

The ester compound represented by general formula (3) according to the present invention can be produced in the same manner as the method for producing the ester compound represented by general formula (1).

Specifically, for the ester compound represented by general formula (3), there are no particular limitations on the starting materials in the production of the ester compound and the method for producing the ester compound. For example, as illustrated by the following reaction formula, a production method in which an ester compound represented by general formula (10) is synthesized by an esterification step of reacting a bisphenol compound represented by general formula (9) and an acid anhydride represented by general formula (6) to synthesize the ester compound represented by general formula (10), and then an ester compound represented by general formula (3) is obtained by a transesterification reaction step of reacting the ester compound represented by general formula (10) and a furan-containing carboxylic acid represented by general formula (8) to obtain the ester compound represented by general formula (3), may be used.

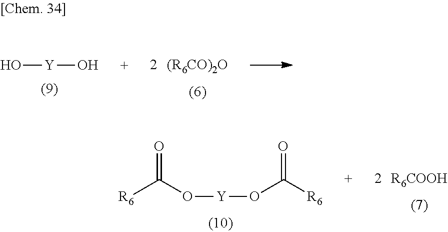

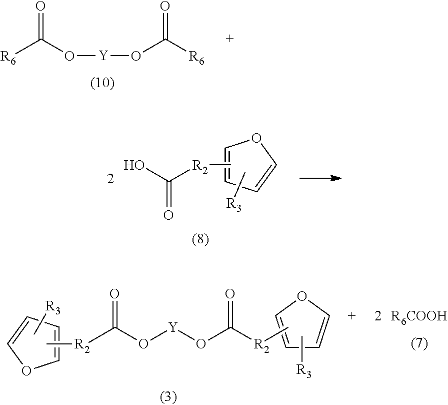

(Y in general formulas (9) and (10) are as defined in general formula (3), R₂ and R₃ in general formula (8) are as defined in general formula (1), and Re in general formula (6), general formula (7), and general formula (10) is as defined in general formula (2).)

### (Esterification Step)

As a method of the reaction in the esterification step in the above production method, a conventionally known esterification reaction method can be used.

Regarding the bisphenol compound represented by general formula (9), specific examples of compounds in the case where Y is represented by general formula (3a) include 4,4′-dihydroxy-3,3′-dimethylbiphenyl, 4,4′-dihydroxy-3,3′,5,5′-tetramethylbiphenyl, and 4,4′-dihydroxy-2, 2′,3,3′5,5′-hexamethylbiphenyl, and specific examples of compounds in the case where Y is represented by general formula (3b) include bisphenol TMC (1,1-bis(4-hydroxyphenyl)-3,3,5-trimethylcyclohexane), 1,1-bis(4-hydroxyphenyl)-3-methylcyclohexane, 1,1-bis(4-hydroxyphenyl)-4-methylcyclohexane, and 1,1-bis(4-hydroxyphenyl)cyclododecane.

Specific examples of the acid anhydride represented by general formula (6) include acetic anhydride and benzoic anhydride.

In the above production method, the amount of the acid anhydride represented by general formula (6) used is preferably in the range of 2.0 to 10.0 mol, more preferably in the range of 2.0 to 8.0 mol, still more preferably in the range of 2.0 to 4.0 mol, relative to 1 mol of the bisphenol compound represented by general formula (9).

The reaction temperature is typically in the range of 50° C. to 150° C., preferably in the range of 80° C. to 140° C. The reaction pressure may be either normal pressure or reduced pressure.

The ester compound represented by general formula (10) can be produced through this esterification step.

Y in general formula (10) is as defined in general formula (3), and preferred forms are also the same.

R₆ in general formula (10) is as defined in general formula (2), and specific examples and preferred forms thereof are also the same.

The ester compound represented by general formula (10) obtained through the esterification step may be used as a raw material for the transesterification reaction step described later. The ester compound may be used as it is contained in the reaction liquid of the esterification reaction, may be used after being purified by removing a carboxylic acid represented by general formula (7) produced as a result of the esterification reaction by distillation, or may be used after being purified by mixing the esterification reaction liquid with a solvent and performing a crystallization operation.

### (Transesterification Reaction Step)

As a method of the reaction in the transesterification reaction step in the above production method, a conventionally known transesterification reaction method can be used.

R₂ and R₃ in general formula (8) are as defined in general formula (1), and preferred forms and specific examples are also the same.

Specific examples of the furan-containing carboxylic acid represented by general formula (8) include 2-furancarboxylic acid, 3-furancarboxylic acid, 2-methyl-3-furancarboxylic acid, and 3-methyl-2-furancarboxylic acid.

The amount of the furan-containing carboxylic acid represented by general formula (8) used is preferably in the range of 2.0 to 10.0 mol, more preferably in the range of 2.0 to 8.0 mol, still more preferably in the range of 2.0 to 4.0 mol, relative to 1 mol of the ester compound represented by general formula (10).

It is preferable to use a base as a catalyst in the reaction between the ester compound represented by general formula (10) and the furan-containing carboxylic acid represented by general formula (8). Specific examples of such a base include organic bases such as amine bases, inorganic alkali metal compounds such as hydroxides, carbonates, and hydrogen carbonate compounds of alkali metals, and organic alkali metal compounds such as salts of alkali metals and alcohols, phenols, or organic carboxylic acids, and also include mixtures thereof, but are not limited thereto.

The reaction is typically carried out in the presence of a solvent. For reasons of, for example, ease of operation in industrial production and improvement in reaction rate, it is preferable to use a reaction solvent in the reaction. The solvent that can be used is not particularly limited as long as it does not distill out of a reaction vessel at the following reaction temperature and is inert to the transesterification reaction. Specific examples include aromatic hydrocarbon ether solvents including alkyl aryl ethers such as phenetole and butyl phenyl ether and diaryl ethers such as diphenyl ether and di-p-tolyl ether, aromatic hydrocarbon solvents such as biphenyl and terphenyl, alkyl-substituted naphthalenes such as diisopropylnaphthalene, aliphatic hydrocarbons such as decalin and kerosene, polyalkylene glycol ethers such as tetraethylene glycol dimethyl ether and diethylene glycol dibutyl ether, and organic solvents such as Therm-S series (manufactured by Nippon Steel Chemical Co., Ltd.), KSK-OIL series (manufactured by Soken Chemical & Engineering Co., Ltd.), and NeoSK-OIL series (manufactured by Soken Chemical & Engineering Co., Ltd.). The amount of the solvent used is not particularly limited as long as the reaction is not hindered, and is typically in the range of 0.5 to 20 times, preferably in the range of 1 to 10 times the amount of the ester compound represented by general formula (10) on a weight basis.

The reaction temperature is typically in the range of 40° C. to 260° C., preferably in the range of 80° C. to 255° C., more preferably in the range of 120° C. to 250° C., still more preferably in the range of 160° C. to 245° C., particularly preferably in the range of 180° C. to 240° C.

The reaction may be carried out under normal pressure conditions, or may be carried out under increased pressure or reduced pressure.

In another embodiment, a process of removing the carboxylic acid represented by general formula (7) produced during the reaction out of the system may be included. The process of removing the produced carboxylic acid represented by general formula (7) from a reaction solution is not particularly limited and can be performed by distilling the produced carboxylic acid represented by general formula (7) together with the solvent system in the reaction solution. The produced carboxylic acid represented by general formula (7) can be removed out of the reaction system by using, for example, an isobaric dropping funnel equipped with a cock, a Dimroth condenser, or a Dean-Stark apparatus.

From the final reaction mixture obtained, the ester compound represented by general formula (3) can be obtained by a known method after completion of the reaction. For example, one possible method is subjecting the reaction mixture to cooling crystallization and performing filtration after the reaction to thereby obtain the target as powder or particles. Other possible methods include adding the reaction mixture to a poor solvent to obtain the target as a precipitate, and adding a solvent to the reaction mixture to cause crystallization and performing filtration to obtain the target as powder or particles.

The ester compound represented by general formula (3) collected by any of the above-described methods can be made into a high-purity product by, for example, standard purification means such as washing with a solvent or water or recrystallization. The solvent that can be used for the crystallization and reslurrying is not particularly limited as long as it is a solvent inert to the ester compound represented by general formula (3), and specific examples include alcohol solvents such as methanol, ethanol, isopropanol, and 1-butanol, carbonyl solvents such as acetic anhydride, acetone, methyl ethyl ketone, methyl isobutyl ketone, cyclopentanone, and cyclohexanone, ether solvents such as tetrahydrofuran, methyl isobutyl ether, methyl isopropyl ether, and diphenyl ether, aromatic nonpolar solvents such as toluene, xylene, and ethylbenzene, and γ-butyrolactone, γ-valerolactone, acetonitrile, dimethylformamide, dimethylacetamide, and N-methylpyrrolidone. Among them, for example, 1-butanol, isopropanol, and diphenyl ether are preferred.

The conditions of the crystallization vary depending on the solvent used; for example, when 1-butanol is used, the amount of the solvent used is in the range of 1 part by weight to 50 parts by weight, more preferably in the range of 2 parts by weight to 30 parts by weight, particularly preferably in the range of 2 to 10 parts by weight, relative to 1 part by weight of the total amount of the composition including the ester compound represented by general formula (3) to be purified and other impurities. The temperature at the time of dissolution is in the range of 50° C. to 250° C., more preferably in the range of 70° C. to 230° C., still more preferably in the range of 80° C. to 200° C., particularly preferably in the range of 90° C. to 180° C. The cooling temperature is in the range of 0° C. to 50° C., more preferably in the range of 10° C. to 40° C., still more preferably in the range of 15° C. to 35° C. The crystallization may be performed under normal pressure conditions, or may be performed under increased pressure.

When any other solvent is used, the conditions can be appropriately changed in view of, for example, the boiling point of the solvent and the solubility of the ester compound represented by general formula (3) to be purified, other impurities, and the composition including them.

The purified product obtained through such a purification process may contain the solvent used, and thus is preferably dried by removing the solvent. One non-limiting example of the method of removing the solvent is to perform heating under normal pressure or reduced pressure to distill off the solvent.

<Crystal of Compound (p-148)>

A crystal of the compound represented by formula (p-148) among the ester compounds according to the present invention is very useful because it can be handled as a solid with crystallinity and is excellent in handleability. The maximum endothermic peak temperature of this crystal determined by differential scanning calorimetry is preferably in the range of 234° C. to 240° C., more preferably in the range of 235° C. to 240° C., particularly preferably in the range of 236° C. to 239° C.

<Method for Producing Crystal of Compound (p-148)>

Compound (p-148) produced by the above-described method for producing the ester compound represented by general formula (3) can be produced by crystallization using solvents including an alcohol solvent such as methanol, ethanol, isopropanol, or 1-butanol, particularly, an alcohol solvent having 1 to 4 carbon atoms, and an ether solvent such as tetrahydrofuran, methyl isobutyl ether, methyl isopropyl ether, or diphenyl ether, particularly, an ether solvent having 4 to 12 carbon atoms.

The alcohol solvent having 1 to 4 carbon atoms is particularly preferably 1-butanol or isopropanol, and the ether solvent having 4 to 12 carbon atoms is particularly preferably diphenyl ether.

For the conditions of the crystallization, the total amount of the alcohol solvent and the ether solvent used is in the range of 1 part by weight to 50 parts by weight, more preferably in the range of 2 parts by weight to 30 parts by weight, particularly preferably in the range of 2 to 10 parts by weight, relative to 1 part by weight of the total amount of the composition including compound (p-148) and other impurities remaining after the reaction.

Regarding the ratio between the alcohol solvent and the ether solvent used, the amount of the ether solvent used is preferably 0.3 to 3.0 times, more preferably 0.5 to 2.5 times, still more preferably 1.0 to 2.2 times, particularly preferably 1.4 to 2.2 times the amount of the alcohol solvent used, on a weight basis.

The temperature at the time of dissolution is in the range of 50° C. to 250° C., more preferably in the range of 70° C. to 230° C., still more preferably in the range of 80° C. to 200° C., particularly preferably in the range of 90° C. to 180° C. The cooling temperature is in the range of 0° C. to 50° C., more preferably in the range of 10° C. to 40° C., still more preferably in the range of 15° C. to 35° C. The crystallization may be performed under normal pressure conditions, or may be performed under increased pressure.

The crystal obtained by the crystallization may contain the solvents used, and thus is preferably dried by removing the solvents. One non-limiting example of the method of removing the solvents is to perform heating under normal pressure or reduced pressure to distill off the solvent.

<Crystal of Compound (p-151)>

A crystal of the compound represented by formula (p-151) among the ester compounds according to the present invention is very useful because it can be handled as a solid with crystallinity and is excellent in handleability.

The maximum endothermic peak temperature of this crystal determined by differential scanning calorimetry is preferably in the range of 180° C. to 188° C., more preferably in the range of 181° C. to 187° C., particularly preferably in the range of 182° C. to 186° C.

<Method for Producing Crystal of Compound (p-151)>

Compound (p-151) produced by the above-described method for producing the ester compound represented by general formula (3) can be produced by crystallization using a solvent including an alcohol solvent such as methanol, ethanol, isopropanol, or 1-butanol, particularly, an alcohol solvent having 1 to 4 carbon atoms.

The alcohol solvent having 1 to 4 carbon atoms is particularly preferably 1-butanol or isopropanol.

For the conditions of the crystallization, the amount of the alcohol solvent used is in the range of 1 part by weight to 50 parts by weight, more preferably in the range of 2 parts by weight to 30 parts by weight, particularly preferably in the range of 2 parts by weight to 10 parts by weight, relative to 1 part by weight of the total amount of the composition including compound (p-151) and other impurities remaining after the reaction.

The temperature at the time of dissolution is in the range of 50° C. to 250° C., more preferably in the range of 70° C. to 230° C., still more preferably in the range of 80° C. to 200° C., particularly preferably in the range of 90° C. to 180° C. The cooling temperature is in the range of 0° C. to 50° C., more preferably in the range of 10° C. to 40° C., still more preferably in the range of 15° C. to 35° C. The crystallization may be performed under normal pressure conditions, or may be performed under increased pressure.

The crystal obtained by the crystallization may contain the solvent used, and thus is preferably dried by removing the solvent. One non-limiting example of the method of removing the solvent is to perform heating under normal pressure or reduced pressure to distill off the solvent.

### <Ester Compound Composition for Resin Raw Material>

The ester compound represented by general formula (3) can also be used in the form of a composition for a resin raw material, the composition further containing an ester compound represented by general formula (2).

The ester compound represented by general formula (2) further contained in the composition for a resin raw material particularly may be an ester compound represented by general formula (10), and this case is more preferred.

In the composition for a resin raw material, the ester compound represented by general formula (2) is preferably contained in an amount of 0.1 to 400 parts by weight, more preferably contained in an amount of 0.1 to 200 parts by weight, still more preferably contained in an amount of 0.1 to 150 parts by weight, even more preferably contained in an amount of 0.1 to 100 parts by weight, particularly preferably contained in an amount of 0.1 to 10 parts by weight, relative to 100 parts by weight of the ester compound represented by general formula (3).

The composition for a resin raw material according to the present invention can be produced by mixing the ester compound represented by general formula (3) and the ester compound represented by general formula (2), which have been produced separately, so as to achieve a desired amount, or can be produced by using the ester compound represented by general formula (2) as an intermediate for producing the ester compound represented by general formula (3) and controlling the reaction rate of the transesterification reaction with the furan-containing carboxylic acid represented by general formula (8) so as to achieve a desired amount.

Such an ester compound composition for a resin raw material that contains the ester compound represented by general formula (3) and the ester compound represented by general formula (2) can be used to produce a cured product by being reacted with the thermosetting compound and/or the compound having a radical polymerizable substituent as in, for example, the curable composition described later. Also in producing an epoxy resin formed by reacting the ester compound represented by general formula (3) described later, the ester compound composition for a resin raw material further containing the ester compound represented by general formula (2) can be used.

### <Epoxy Resin Formed by Reacting Ester Compound Represented by General Formula (3)>

The ester compound represented by general formula (3) according to the present invention can be reacted with an aromatic diglycidyl ether compound (e.g., a compound derived by glycidylating a hydroxy group of hydroquinone, resorcinol, catechol, the bisphenol compound represented by general formula (5), or the like) to provide an epoxy resin. To facilitate the progress of molecular weight increase in a state where epoxy groups are present at molecular ends, the reaction is preferably carried out with the amount used being in the range of (epoxy group):(ester group)=1 to 1.2:1 in an equivalent mixing ratio.

The epoxy resin formed by reacting the ester compound represented by general formula (3) may also be an epoxy resin derived from reaction with a mixture containing an aromatic dihydroxy compound and epihalohydrin or an epoxy resin derived from reaction with an epoxy group-containing phenoxy resin formed by polymerizing an aromatic dihydroxy compound and epihalohydrin.

That is, the epoxy resin formed by reacting the ester compound represented by general formula (3) may be an epoxy resin formed by reacting the ester compound represented by general formula (3) with any one selected from an aromatic diglycidyl ether compound, a mixture containing an aromatic dihydroxy compound and epihalohydrin, and an epoxy group-containing phenoxy resin formed by polymerizing an aromatic dihydroxy compound and epihalohydrin.

### (Method for Producing Epoxy Resin Formed by Reacting Ester Compound Represented by General Formula (3))

A catalyst may be used in the synthesis of the epoxy resin formed by reacting the ester compound represented by general formula (3), and the catalyst may be any compound having a catalytic ability to promote the reaction between epoxy groups and ester groups. Examples include tertiary amines, cyclic amines, imidazoles, organophosphorus compounds, and quaternary ammonium salts.

Specific examples of tertiary amines include triethylamine, tri-n-propylamine, tri-n-butylamine, triethanolamine, benzyldimethylamine, pyridine, and 4-(dimethylamino)pyridine.

Specific examples of cyclic amines include 1,4-diazabicyclo[2,2,2]octane, 1,8-diazabicyclo[5,4,0]-7-undecene, and 1,5-diazabicyclo[4,3,0]-5-nonene.

Specific examples of imidazoles include 2-methylimidazole, 2-ethyl-4-methylimidazole, and 2-phenylimidazole.

Specific examples of organophosphorus compounds include tri-n-propylphosphine, tri-n-butylphosphine, triphenylphosphine, tris(p-tolyl)phosphine, tricyclohexylphosphine, tri (tert-butyl)phosphine, tris(p-methoxyphenyl)phosphine, tetramethylphosphonium bromide, tetramethylphosphonium iodide, tetramethylphosphonium hydroxide, tetrabutylphosphonium hydroxide, trimethylcyclohexylphosphonium chloride, trimethylcyclohexylphosphonium bromide, trimethylbenzylphosphonium chloride, trimethylbenzylphosphonium bromide, tetraphenylphosphonium bromide, triphenylmethylphosphonium bromide, triphenylmethylphosphonium iodide, triphenylethylphosphonium chloride, triphenylethylphosphonium bromide, triphenylethylphosphonium iodide, triphenylbenzylphosphonium chloride, and triphenylbenzylphosphonium bromide.

Among the catalysts listed above, 4-(dimethylamino)pyridine, 1,4-diazabicyclo[2,2,2]octane, 1,8-diazabicyclo[5,4,0]-7-undecene, 1,5-diazabicyclo[4,3,0]-5-nonene, 2-ethyl-4-methylimidazole, tris(p-tolyl)phosphine, tricyclohexylphosphine, tri (tert-butyl)phosphine, and tris(p-methoxyphenyl) phosphine are preferred, and in particular, 4-(dimethylamino)pyridine, 1,8-diazabicyclo[5,4,0]-7-undecene, 1,5-diazabicyclo[4,3,0]-5-nonene, and 2-ethyl-4-methylimidazole are preferred. The catalysts may be used alone or in combination of two or more.

The amount of the above catalysts used is in the range of 0.001 to 3 wt % relative to the amount of reaction substrate used in the reaction for obtaining the epoxy resin formed by reacting the ester compound represented by general formula (3). When such a compound is used as a catalyst, it may remain as a catalyst residue in the resulting curable composition to deteriorate the insulating characteristics of a printed wiring board or shorten the pot life of the composition, and thus when a compound containing nitrogen is used as a catalyst, the content of nitrogen in the curable composition is preferably 2000 ppm or less, more preferably 1000 ppm or less. When a compound containing phosphorus is used as a catalyst, the content of phosphorus in the curable composition is preferably 2000 ppm or less, more preferably 1000 ppm or less.

In the process of the synthesis reaction during the production of the epoxy resin formed by reacting the ester compound represented by general formula (3) according to the present invention, a solvent for the reaction may be used, and the solvent may be any solvent that dissolves the epoxy resin. Examples include aromatic hydrocarbon solvents, ketone solvents, amide solvents, and glycol ether solvents. The solvents may be used alone or in combination of two or more.

Specific examples of aromatic hydrocarbon solvents include benzene, toluene, and xylene. Specific examples of ketone solvents include acetone, methyl ethyl ketone (MEK), methyl isobutyl ketone, 2-heptanone, 4-heptanone, 2-octanone, cyclohexanone, acetylacetone, and dioxane.

Specific examples of amide solvents include formamide, N-methylformamide, N, N-dimethylformamide, acetamide, N-methylacetamide, N, N-dimethylacetamide, 2-pyrolidone, and N-methylpyrrolidone.

Specific examples of glycol ether solvents include ethylene glycol monomethyl ether, ethylene glycol monoethyl ether, ethylene glycol mono-n-butyl ether, ethylene glycol dimethyl ether, ethylene glycol monoethyl ether acetate, diethylene glycol monomethyl ether, diethylene glycol monoethyl ether, diethylene glycol mono-n-butyl ether, diethylene glycol dimethyl ether, diethylene glycol monoethyl ether acetate, propylene glycol monomethyl ether, propylene glycol mono-n-butyl ether, and propylene glycol monomethyl ether acetate.

The solids concentration in the synthesis reaction during the production of the epoxy resin is preferably 10 to 95 wt %. If a highly viscous product is produced during the reaction, the solvent can be additionally added to continue the reaction. After completion of the reaction, the solvent can be removed or further added as needed.

The polymerization reaction in the production of the epoxy resin is carried out at a reaction temperature at which the catalyst used does not decompose. If the reaction temperature is too high, the catalyst may decompose to stop the reaction, or the produced epoxy resin may deteriorate. By contrast, if the temperature is too low, the reaction may fail to proceed sufficiently. For these reasons, the reaction temperature is preferably 50° C. to 250° C., more preferably 120° C. to 230° C. The reaction time is typically 1 to 12 hours, preferably 3 to 10 hours. When a low-boiling solvent such as acetone or methyl ethyl ketone is used, the reaction temperature can be secured by carrying out the reaction under high pressure using an autoclave.

The ester compound represented by general formula (3) is included in the ester compound represented by general formula (1) and can be used similarly, and therefore, the ester compound represented by general formula (1) will be described below.

Hereinafter, the invention in which the ester compound represented by general formula (1) is replaced with the ester compound represented by general formula (3) is also disclosed.

The curable composition according to the present invention contains the ester compound represented by general formula (1), and the thermosetting compound and/or the compound having a radical polymerizable substituent. That is, forms of the curable composition according to the present invention include a form in which the ester compound represented by general formula (1) and the thermosetting compound are contained, a form in which the ester compound represented by general formula (1) and the compound having a radical polymerizable substituent are contained, and a form in which the ester compound represented by general formula (1), and the thermosetting compound and the compound having a radical polymerizable substituent are contained.

To compensate for deficiencies in or further improve the physical properties and characteristics of the curable composition according to the present invention and the cured product obtained therefrom, those skilled in the art to which the present invention pertains can appropriately and suitably select one from the above forms, select or change the compound for use, and adjust the amount of the compound used, etc. within the scope disclosed by the present invention and the obvious scope.

Among the compounds encompassed within the scope of the ester compound represented by general formula (1) used in the curable composition according to the present invention, one compound may be used alone, or two or more compounds can be used in combination.

The ester compound represented by general formula (1) used in the curable composition according to the present invention can also be used in the form of a curable composition further containing the ester compound represented by general formula (2).

In this case, the ester compound represented by general formula (2) is preferably contained in an amount of 0.1 to 400 parts by weight, more preferably contained in an amount of 0.1 to 200 parts by weight, still more preferably contained in an amount of 0.1 to 150 parts by weight, even more preferably contained in an amount of 0.1 to 100 parts by weight, particularly preferably contained in an amount of 0.1 to 10 parts by weight, relative to 100 parts by weight of the ester compound represented by general formula (1).

The curable composition can be produced by mixing the ester compound represented by general formula (1) and the ester compound represented by general formula (2), which have been produced separately, so as to achieve a desired amount, or can be produced by using the ester compound represented by general formula (2) as an intermediate for producing the ester compound represented by general formula (1) and controlling the reaction rate of the transesterification reaction with the furan-containing carboxylic acid represented by general formula (8) so as to achieve a desired amount.

### <Thermosetting Compound>

The thermosetting compound used in the curable composition according to the present invention may be a conventionally known thermosetting compound, and is specifically, for example, at least one compound selected from the group consisting of an epoxy resin, a benzoxazine compound, a benzoxazine resin, a phenol resin, a bismaleimide compound, and a maleimide resin.

The epoxy resin, the benzoxazine compound, the benzoxazine resin, the phenol resin, the bismaleimide compound, and the maleimide resin may each be any of compounds including conventionally known compounds.

### (Epoxy Resin)

The epoxy resin also includes glycidyl ether compounds, aromatic diglycidyl ether compounds (e.g., a compound derived by glycidylating a hydroxy group of hydroquinone, resorcinol, catechol, the bisphenol compound represented by general formula (5), or the like), epoxy group-containing phenoxy resins formed by polymerizing an aromatic dihydroxy compound (e.g., hydroquinone, resorcinol, catechol, or the bisphenol compound represented by general formula (5)) and epihalohydrin with or without an active ester curing agent, and the like, and any epoxy resins including conventionally known compounds can be used. An epoxy resin formed by reacting the ester compound represented by general formula (1) according to the present invention can also be used as such an active ester curing agent, and this is preferably used. These can be used alone or as a mixture of two or more.

The conventionally known epoxy resin for use preferably has two or more epoxy groups in its molecule, and for example, various epoxy resins such as bisphenol A-type epoxy resins, bisphenol F-type epoxy resins, bisphenol S-type epoxy resins, bisphenol AF-type epoxy resins, bisphenol Z-type epoxy resins, naphthalene-type epoxy resins, phenol novolac-type epoxy resins, cresol novolac-type epoxy resins, phenol aralkyl-type epoxy resins, biphenyl-type epoxy resins, triphenylmethane-type epoxy resins, dicyclopentadiene-type epoxy resins, and phenoxy resins can be used.

When the epoxy resin formed by reacting the ester compound represented by general formula (1) and another epoxy resin are used in combination, the amount of the other epoxy resin in all the epoxy resin components is preferably 1 wt % or more, more preferably 5 wt % or more, still more preferably 10 wt % or more, while it is preferably 99 wt % or less, more preferably 95 wt % or less, still more preferably 90 wt % or less. When the proportion of the other epoxy resin is equal to or greater than the lower limit, the effect of improving physical properties by adding the other epoxy resin can be sufficiently obtained. On the other hand, when the proportion of the other epoxy resin is equal to or less than the upper limit, the effect of the epoxy resin according to the present invention is fully exhibited, which is preferred from the viewpoint of providing film-forming properties.

When a solvent is contained, the amounts of the epoxy resin formed by reacting the ester compound represented by general formula (1) and the other epoxy resin are based on the amount excluding the amount of the solvent.

### (Epoxy Resin Formed by Reacting Ester Compound Represented by General Formula (1))

The epoxy resin formed by reacting the ester compound represented by general formula (1) according to the present invention can be obtained by reacting the ester compound represented by general formula (1) with, for example, an aromatic diglycidyl ether compound (e.g., a compound derived by glycidylating a hydroxy group of hydroquinone, resorcinol, catechol, the bisphenol compound represented by general formula (5), or the like). To facilitate the progress of molecular weight increase in a state where epoxy groups are present at molecular ends, the reaction is preferably carried out with the amount used being in the range of (epoxy group):(ester group)=1 to 1.2:1 in an equivalent mixing ratio.

Other methods include reacting the ester compound represented by general formula (1) with a mixture containing an aromatic dihydroxy compound and epihalohydrin, and reacting the ester compound represented by general formula (1) with an epoxy group-containing phenoxy resin formed by polymerizing an aromatic dihydroxy compound and epihalohydrin.

That is, the epoxy resin formed by reacting the ester compound represented by general formula (1) may be an epoxy resin formed by reacting the ester compound represented by general formula (1) with any one selected from an aromatic diglycidyl ether compound, a mixture containing an aromatic dihydroxy compound and epihalohydrin, and an epoxy group-containing phenoxy resin formed by polymerizing an aromatic dihydroxy compound and epihalohydrin.

A catalyst may be used in the synthesis of the epoxy resin formed by reacting the ester compound represented by general formula (1), and the catalyst may be any compound having a catalytic ability to promote the reaction between epoxy groups and ester groups. Examples include tertiary amines, cyclic amines, imidazoles, organophosphorus compounds, and quaternary ammonium salts.

Specific examples of tertiary amines include triethylamine, tri-n-propylamine, tri-n-butylamine, triethanolamine, benzyldimethylamine, pyridine, and 4-(dimethylamino)pyridine.

Specific examples of cyclic amines include 1,4-diazabicyclo[2,2,2]octane, 1,8-diazabicyclo[5,4,0]-7-undecene, and 1,5-diazabicyclo[4,3,0]-5-nonene.

Specific examples of imidazoles include 2-methylimidazole, 2-ethyl-4-methylimidazole, and 2-phenylimidazole.

Specific examples of organophosphorus compounds include tri-n-propylphosphine, tri-n-butylphosphine, triphenylphosphine, tris(p-tolyl) phosphine, tricyclohexylphosphine, tri (tert-butyl)phosphine, tris(p-methoxyphenyl)phosphine, tetramethylphosphonium bromide, tetramethylphosphonium iodide, tetramethylphosphonium hydroxide, tetrabutylphosphonium hydroxide, trimethylcyclohexylphosphonium chloride, trimethylcyclohexylphosphonium bromide, trimethylbenzylphosphonium chloride, trimethylbenzylphosphonium bromide, tetraphenylphosphonium bromide, triphenylmethylphosphonium bromide, triphenylmethylphosphonium iodide, triphenylethylphosphonium chloride, triphenylethylphosphonium bromide, triphenylethylphosphonium iodide, triphenylbenzylphosphonium chloride, and triphenylbenzylphosphonium bromide.

Among the catalysts listed above, 4-(dimethylamino)pyridine, 1,4-diazabicyclo[2,2,2]octane, 1,8-diazabicyclo[5,4,0]-7-undecene, 1,5-diazabicyclo[4,3,0]-5-nonene, 2-ethyl-4-methylimidazole, tris(p-tolyl) phosphine, tricyclohexylphosphine, tri (tert-butyl)phosphine, and tris(p-methoxyphenyl) phosphine are preferred, and in particular, 4-(dimethylamino)pyridine, 1,8-diazabicyclo[5,4,0]-7-undecene, 1,5-diazabicyclo[4,3,0]-5-nonene, and 2-ethyl-4-methylimidazole are preferred. The catalysts may be used alone or in combination of two or more.

The amount of the above catalysts used is in the range of 0.001 to 3 wt % relative to the amount of reaction substrate used in the reaction for obtaining the epoxy resin formed by reacting the ester compound represented by general formula (1). When such a compound is used as a catalyst, it may remain as a catalyst residue in the resulting curable composition to deteriorate the insulating characteristics of a printed wiring board or shorten the pot life of the composition, and thus when a compound containing nitrogen is used as a catalyst, the content of nitrogen in the curable composition is preferably 2000 ppm or less, more preferably 1000 ppm or less. When a compound containing phosphorus is used as a catalyst, the content of phosphorus in the curable composition is preferably 2000 ppm or less, more preferably 1000 ppm or less.

In the process of the synthesis reaction during the production of the epoxy resin formed by reacting the ester compound represented by general formula (1) according to the present invention, a solvent for the reaction may be used, and the solvent is not limited as long as it dissolves the epoxy resin. Examples include aromatic hydrocarbon solvents, ketone solvents, amide solvents, and glycol ether solvents. The solvents may be used alone or in combination of two or more.

Specific examples of aromatic hydrocarbon solvents include benzene, toluene, and xylene. Specific examples of ketone solvents include acetone, methyl ethyl ketone (MEK), methyl isobutyl ketone, 2-heptanone, 4-heptanone, 2-octanone, cyclohexanone, acetylacetone, and dioxane.

Specific examples of amide solvents include formamide, N-methylformamide, N, N-dimethylformamide, acetamide, N-methylacetamide, N, N-dimethylacetamide, 2-pyrolidone, and N-methylpyrrolidone.

Specific examples of glycol ether solvents include ethylene glycol monomethyl ether, ethylene glycol monoethyl ether, ethylene glycol mono-n-butyl ether, ethylene glycol dimethyl ether, ethylene glycol monoethyl ether acetate, diethylene glycol monomethyl ether, diethylene glycol monoethyl ether, diethylene glycol mono-n-butyl ether, diethylene glycol dimethyl ether, diethylene glycol monoethyl ether acetate, propylene glycol monomethyl ether, propylene glycol mono-n-butyl ether, and propylene glycol monomethyl ether acetate.

The solids concentration in the synthesis reaction during the production of the epoxy resin is preferably 10 to 95 wt %. If a highly viscous product is produced during the reaction, the solvent can be additionally added to continue the reaction. After completion of the reaction, the solvent can be removed or further added as needed.

The polymerization reaction in the production of the epoxy resin is carried out at a reaction temperature at which the catalyst used does not decompose. If the reaction temperature is too high, the catalyst may decompose to stop the reaction, or the produced epoxy resin may deteriorate. By contrast, if the temperature is too low, the reaction may fail to proceed sufficiently. For these reasons, the reaction temperature is preferably 50° C. to 250° C., more preferably 120° C. to 230° C. The reaction time is typically 1 to 12 hours, preferably 3 to 10 hours. When a low-boiling solvent such as acetone or methyl ethyl ketone is used, the reaction temperature higher than the boiling point under normal pressure can be secured by carrying out the reaction under high pressure using an autoclave.

### <Compound Having Radical Polymerizable Substituent>

The compound having a radical polymerizable substituent used in the curable composition according to the present invention may be a conventionally known compound, and is specifically, for example, at least one compound selected from the group consisting of a diallyl phthalate resin, a diallyl phthalate compound, a polyphenylene ether resin having a radical polymerizable substituent, and a vinyl compound.

The diallyl phthalate resin, the diallyl phthalate compound, the polyphenylene ether resin having a radical polymerizable substituent, and the vinyl compound may be any of compounds including conventionally known compounds.

<Content of Thermosetting Compound and/or Compound Having Radical Polymerizable Substituent>

In the curable composition according to the present invention, the content of the thermosetting compound and/or the compound having a radical polymerizable substituent is preferably in the range of 50 to 500 parts by weight, more preferably in the range of 50 to 400 parts by weight, still more preferably in the range of 50 to 300 parts by weight, particularly preferably in the range of 50 to 200 parts by weight, relative to 100 parts by weight of the ester compound represented by general formula (1).

That is, among the forms of the curable composition according to the present invention, in the case of the form in which the ester compound represented by general formula (1) and the thermosetting compound are contained, it means that the thermosetting compound is contained in the above range relative to 100 parts by weight of the ester compound represented by general formula (1); in the case of the form in which the ester compound represented by general formula (1) and the compound having a radical polymerizable substituent are contained, it means that the compound having a radical polymerizable substituent is contained in the above range relative to 100 parts by weight of the ester compound represented by general formula (1); and in the case of the form in which the ester compound represented by general formula (1), and the thermosetting compound and the compound having a radical polymerizable substituent are contained, it means that the thermosetting compound and the compound having a radical polymerizable substituent, in total, are contained in the above range relative to 100 parts by weight of the ester compound represented by general formula (1).

When the ester compound represented by general formula (2) is used in combination, the content relative to 100 parts by weight of the total amount of the ester compound represented by general formula (1) and the ester compound represented by general formula (2) is employed instead.

### <Additive>

The curable composition according to the present invention may optionally further contain various additives such as ultraviolet inhibitors, antioxidants, coupling agents, plasticizers, fluxes, flame retardants, colorants, dispersants, emulsifiers, elasticity reducers, diluents, antifoaming agents, ion trapping agents, inorganic fillers, and organic fillers. The types and amounts of additives used can be appropriately adjusted depending on the intended use.

### <Curing Agent>

The curable composition according to the present invention may further contain a curing agent.

In the case where an epoxy resin is used as a component of the curable composition according to the present invention, the curing agent includes a substance that contributes to the crosslinking reaction and/or chain extension reaction between epoxy groups of the epoxy resin. In this case, even a substance that is generally called a “curing accelerator” is included in the curing agent if it contributes to the crosslinking reaction and/or chain extension reaction between epoxy groups of the epoxy resin.

The amount of the curing agent used is preferably 0.1 to 100 parts by weight, more preferably 80 parts by weight or less, still more preferably 60 parts by weight or less, relative to 100 parts by weight of the total amount of the ester compound represented by general formula (1) and the thermosetting resin or the compound having a radical polymerizable substituent used.

The curing agent for use is not particularly limited, and any substance generally known as a curing agent for a thermosetting resin or a compound having a radical polymerizable substituent can be used.

Specific examples of the curing agent used in the curable composition according to the present invention in the case where an epoxy resin is used include phenolic curing agents, amide curing agents, imidazoles, and active ester curing agents, which are preferred from the viewpoint of enhancing heat resistance. Examples of phenolic curing agents, amide curing agents, imidazoles, active ester curing agents, and other usable curing agents will be given below.

### (Phenolic Curing Agent)

Using a phenolic curing agent as the curing agent is preferred from the viewpoint of improving the handleability of the curable composition to be obtained and the heat resistance of the cured product after curing. Specific examples of the phenolic curing agent include bisphenol A, bisphenol F, 4,4′-dihydroxydiphenylmethane, 4,4′-dihydroxydiphenyl ether, 1,4-bis(4-hydroxyphenoxy)benzene, 1,3-bis(4-hydroxyphenoxy)benzene, 4,4′-dihydroxydiphenyl sulfide, 4,4′-dihydroxydiphenyl ketone, 4,4′-dihydroxydiphenyl sulfone, 4,4′-dihydroxybiphenyl, 2,2′-dihydroxybiphenyl, 10-(2,5-dihydroxyphenyl)-10H-9-oxa-10-phosphaphenanthrene-10-oxide, phenol novolac, bisphenol A novolac, o-cresol novolac, m-cresol novolac, p-cresol novolac, xylenol novolac, poly-p-hydroxystyrene, hydroquinone, resorcin, catechol, t-butylcatechol, t-butylhydroquinone, phloroglucinol, pyrogallol, t-butyl pyrogallol, allylated pyrogallol, polyallylated pyrogallol, 1,2,4-benzenetriol, 2,3,4-trihydroxybenzophenone, 1,2-dihydroxynaphthalene, 1,3-dihydroxynaphthalene, 1,4-dihydroxynaphthalene, 1,5-dihydroxynaphthalene, 1,6-dihydroxynaphthalene, 1,7-dihydroxynaphthalene, 1,8-dihydroxynaphthalene, 2,3-dihydroxynaphthalene, 2,4-dihydroxynaphthalene, 2,5-dihydroxynaphthalene, 2,6-dihydroxynaphthalene, 2,7-dihydroxynaphthalene, 2,8-dihydroxynaphthalene, allylated products or polyallylated products of these dihydroxynaphthalenes, allylated bisphenol A, allylated bisphenol F, allylated phenol novolac, and allylated pyrogallol.

The phenolic curing agents listed above may be used alone or as a mixture of two or more in any combination and ratio. When the curing agent is a phenolic curing agent, it is preferably used such that the equivalent ratio of functional groups in the curing agent to epoxy groups in the epoxy resin is in the range of 0.8 to 1.5. Within this range, unreacted epoxy groups and functional groups of the curing agent are less likely to remain, which is preferred.

### (Amide Curing Agent)

Using an amide curing agent as the curing agent is preferred from the viewpoint of improvement in, for example, heat resistance. The use of an amide curing agent as the curing agent is preferred from the viewpoint of providing a curable composition with improved heat resistance. Examples of the amide curing agent include dicyandiamide and derivatives thereof, and polyamide resins. Specific examples of the amide curing agent include “LUCKAMIDE” N-153-IM-65, EA-330, and TD-960 (manufactured by DIC Corporation).

The amide curing agents listed above may be used alone or as a mixture of two or more in any combination and ratio. The amide curing agent is preferably used in an amount in the range of 0.1 to 20 wt % relative to the total amount of the epoxy resin and the amide curing agent used in the curable composition.

### (Imidazole)

Using an imidazole as the curing agent is preferred from the viewpoint of sufficiently progressing the curing reaction and improving heat resistance. Examples of the imidazole include 2-phenylimidazole, 2-ethyl-4 (5)-methylimidazole, 2-phenyl-4-methylimidazole, 1-benzyl-2-methylimidazole, 1-benzyl-2-phenylimidazole, 1-cyanoethyl-2-undecylimidazole, 1-cyano-2-phenylimidazole, 1-cyanoethyl-2-undecylimidazole trimellitate, 1-cyanoethyl-2-phenylimidazolium trimellitate, 2,4-diamino-6-[2′-methylimidazolyl-(1′)]-ethyl-s-triazine, 2,4-diamino-6-[2′-ethyl-4′-methylimidazolyl-(1′)]-ethyl-s-triazine, isocyanuric acid adducts of 2,4-diamino-6-[2′-methylimidazolyl-(1′)]-ethyl-s-triazine, isocyanuric acid adducts of 2-phenylimidazole, 2-phenyl-4,5-dihydroxymethylimidazole, 2-phenyl-4-methyl-5-hydroxymethylimidazole, and adducts of epoxy resins and these imidazoles. Imidazoles have catalytic ability and thus are generally classifiable as curing accelerators described later, but in the present invention, they are classified as curing agents.

The imidazoles listed above may be used alone or as a mixture of two or more in any combination and ratio. The imidazole is preferably used in an amount in the range of 0.1 to 20 wt % relative to the total amount of the epoxy resin and the imidazole used in the curable composition.

### (Active Ester Curing Agent)

Using an active ester curing agent as the curing agent is preferred from the viewpoint of providing a cured product with lower water absorbency. The active ester curing agent is preferably a compound having two or more highly reactive ester groups in one molecule, such as a phenol ester, a thiophenol ester, an N-hydroxyamine ester, or an ester of a heterocyclic hydroxy compound, and in particular, a phenol ester derived by reacting a carboxylic acid compound and an aromatic compound having a phenolic hydroxy group is more preferred. Specific examples of the carboxylic acid compound include benzoic acid, acetic acid, succinic acid, maleic acid, itaconic acid, phthalic acid, isophthalic acid, terephthalic acid, and pyromellitic acid. Examples of the aromatic compound having a phenolic hydroxy group include catechol, 1,5-dihydroxynaphthalene, 1,6-dihydroxynaphthalene, 2,6-dihydroxynaphthalene, dihydroxybenzophenone, trihydroxybenzophenone, tetrahydroxybenzophenone, phloroglucin, benzenetriol, dicyclopentadienyl diphenol, and phenol novolac. The ester compound represented by general formula (2) according to the present invention can also be used.

The active ester curing agents listed above may be used alone or as a mixture of two or more in any combination and ratio. The active ester curing agent is preferably used such that the equivalent ratio of active ester groups in the curing agent to epoxy groups in the epoxy resin used in the curable composition is in the range of 0.2 to 2.0.

### (Other Curing Agents)

Examples of curing agents that can be used in the curable composition according to the present invention, other than phenolic curing agents, amide curing agents, and imidazoles, include amine curing agents (excluding tertiary amines), acid anhydride curing agents, tertiary amines, organic phosphines, phosphonium salts, tetraphenylboron salts, organic acid dihydrazides, boron halide-amine complexes, polymercaptan curing agents, isocyanate curing agents, and blocked isocyanate curing agents. The other curing agents listed above may be used alone or as a mixture of two or more in any combination and ratio.

Examples of curing agents that can be used when the compound having a radical polymerizable group is used include imidazoles, tertiary amines, quaternary ammonium salts, boron trifluoride-amine complexes, organophosphines, ionic catalysts such as organophosphonium salts, organic peroxides such as di-t-butyl peroxide, dilauroyl peroxide, dibenzoyl peroxide, dicumyl peroxide, and t-butyl peroxybenzoate, and radical polymerization initiators such as hydroperoxide and azoisobutyronitrile.

In the curable composition according to the present invention, when the epoxy resin formed by reacting the ester compound represented by general formula (1) and another compound (a thermosetting compound other than the epoxy resin formed by reacting the ester compound represented by general formula (1) or the compound having a radical polymerizable substituent) are used as components of the thermosetting compound, the amount of the other compound in all the epoxy resin components as solid contents is preferably 1 wt % or more, more preferably 5 wt % or more, still more preferably 10 wt % or more, while it is preferably 99 wt % or less, more preferably 95 wt % or less, still more preferably 90 wt % or less. When the proportion of the other compound is equal to or greater than the lower limit, the effect of improving physical properties by adding the other compound can be sufficiently obtained. On the other hand, when the proportion of the other compound is equal to or less than the upper limit, the effect of the epoxy resin according to the present invention is fully exerted, which is preferred from the viewpoint of providing film-forming properties.

### <Solvent>

The curable composition according to the present invention may be diluted by further adding a solvent in order to appropriately adjust the viscosity of the curable composition during handling in film formation. In the curable composition according to the present invention, the solvent is used to ensure handleability and workability in forming of the curable composition, and the amount of the solvent used is not particularly limited.

Examples of solvents that can be contained in the curable composition according to the present invention include ketones such as acetone, methyl ethyl ketone (MEK), methyl isobutyl ketone, and cyclohexanone, esters such as ethyl acetate, ethers such as ethylene glycol monomethyl ether, amides such as N, N-dimethylformamide and N, N-dimethylacetamide, alcohols such as methanol and ethanol, alkanes such as hexane and cyclohexane, and aromatics such as toluene and xylene. The solvents listed above may be used alone or as a mixture of two or more in any combination and ratio.

### <Cured Product>

A cured product obtained by curing the curable composition according to the present invention is excellent in dielectric properties and heat resistance. The term “curing” as used herein refers to intentional curing of an epoxy resin composition using, for example, heat and/or light, and the degree of curing may be controlled according to the desired physical properties and the intended use. The degree of progress may be full cure or partial cure and is not particularly limited, and the reaction rate of curing reaction is typically 5% to 95%.

### <Curing Method>

The method of curing the curable composition according to the present invention varies depending on the components in the curable composition and the amounts thereof, and heating conditions at 80° C. to 280° C. for 60 to 360 minutes are typically employed. This heating is preferably performed in a two-stage process including primary heating at 80° C. to 160° C. for 10 to 90 minutes and secondary heating at 120° C. to 200° C. for 60 to 150 minutes, and in the case of a formulation having a glass transition temperature (Tg) higher than the temperature of secondary heating, it is preferable to further perform tertiary heating at 150° C. to 280° C. for 60 to 120 minutes. Performing secondary heating and tertiary heating in this manner is preferred from the viewpoint of reducing poor curing and solvent residues.

When a partially cured resin product is produced, it is preferable to allow the curing reaction of the curable composition to proceed by means such as heating to the extent that the shape of the product can be maintained. When the curable composition contains a solvent, most of the solvent is typically removed by means such as heating, decompression, or air drying, but 5 mass % or less of the solvent may be left behind in the partially cured resin product.

### <Applications>

The epoxy resin formed by reacting the ester compound represented by general formula (1) according to the present invention has excellent film-forming properties. Thus, the epoxy resin is applicable to various fields, including adhesives, coatings, materials for civil engineering and construction, and insulating materials for electric and electronic components, and is useful particularly as an insulating casting, a laminating material, a sealing materials, etc. in the electric and electronic fields.

Examples of applications of the epoxy resin formed by reacting the ester compound represented by general formula (1) according to the present invention, the curable composition according to the present invention, and the cured product thereof include film adhesives, liquid adhesives, composite materials, coatings, construction materials for civil engineering, insulating materials for electric and electronic components, multilayer printed circuit boards, laminates for electric and electronic circuits such as capacitors, semiconductor sealing materials, underfill materials, inter-chip fills for 3D-LSI, insulating sheets, prepregs, heat dissipation substrates, and insulating castings, but are not limited thereto.

### (Laminate for Electric and Electronic Circuit)

The curable composition according to the present invention is suitable for use in applications of laminates for electric and electronic circuits as described above. In the present invention, a “laminate for an electric and electronic circuit” is a laminate of a layer containing the curable composition according to the present invention and a conductive metal layer, and is used as a concept including, for example, a capacitor even if it is not an electric and electronic circuit as long as it is a laminate of a layer containing the curable composition according to the present invention and a conductive metal layer. In the laminate for an electric and electronic circuit, two or more curable composition layers may be formed, and the curable composition according to the present invention only needs to be used in at least one layer. Furthermore, two or more conductive metal layers may be formed.

In the laminate for an electric and electronic circuit, the thickness of the curable composition layer is typically about 10 to 200 μm. The thickness of the conductive metal layer is typically about 0.2 to 70 μm.

### (Conductive Metal)

Examples of the conductive metal in the laminate for an electric and electronic circuit include metals such as copper and aluminum and alloys containing such metals. In the present invention, the conductive metal layer of the laminate for an electric and electronic circuit may be a metal foil of such a metal or a metal layer formed by plating or sputtering.

### (Method of Manufacturing Laminate for Electric and Electronic Circuit)

Examples of the method of manufacturing the laminate for an electric and electronic circuit in the present invention include methods as described below.
(1) A nonwoven fabric, a cloth, or the like formed of an inorganic and/or organic fiber material such as glass fiber, polyester fiber, aramid fiber, cellulose, or nanofiber cellulose is impregnated with the curable composition according to the present invention to form a prepreg, on which a conductive metal foil and/or a conductive metal layer formed by plating is provided, and then a circuit is formed using a photoresist or the like. A required number of such layers are stacked to form a laminate.
(2) The prepreg in (1) is used as a core material, and a curable composition layer and a conductive metal layer are laminated on (one surface or both surfaces of) the core material. The curable composition layer may contain an organic and/or inorganic filler.
(3) Without using a core material, a curable composition layer and a conductive metal layer alone are alternately laminated to form a laminate for an electric and electronic circuit.

According to the present invention, a cured product excellent in heat resistance and dielectric properties can be provided.

Thus, the curable composition according to the present invention is applicable to various fields, including film adhesives, liquid adhesives, composite materials, coatings, construction materials for civil engineering, insulating materials for electric and electronic components, multilayer printed circuit boards, laminates for electric and electronic circuits such as capacitors, semiconductor sealing materials, underfill materials, inter-chip fills for 3D-LSI, insulating sheets, prepregs, heat dissipation substrates, and insulating castings, and is useful as an insulating casting, a laminating material, a sealing material, etc, particularly in the electric and electronic fields.

### EXAMPLES

The present invention will now be described more specifically with reference to Examples.

### <Analysis Methods>

### 1. Reaction Solution Composition and Purity Analysis (Ultra-Fast Liquid Chromatography: UFLC)

An ester compound synthesized in Example in an amount of 0.01 g was weighed in a 50 mL measuring flask and diluted with acetonitrile.

The prepared sample was subjected to the following purity analysis by high-performance liquid chromatography. Purity analysis (analysis values are expressed in area percentage)

Measuring apparatus: high-performance liquid chromatography analyzer Prominence UFLC (manufactured by Shimadzu Corporation)
Pump: LC-20AD
Column oven: CTO-20A
Detector: SPD-20A
Column: HALO-C18 (inner diameter 3 mm, length 75 mm)
Oven temperature: 50° C.
Flow rate: 0.7 mL/min.
Mobile phase: (A) 0.1 vol % aqueous acetic acid solution, (B) acetonitrile
Gradient conditions: (A) vol % (time from start of analysis) 30% (2.0 min.)→100% (15.0 min.)→100% (18.0 min.)
Sample injection volume: 7 μL
Detection wavelength: 254 nm

### 2. Curing Properties Evaluation

The curing properties evaluation of a synthesized epoxy resin composition was performed by differential scanning calorimetry (DSC) under the following operating conditions. The exothermic peak temperature was determined as a curing temperature.

### [Measurement Conditions]

Apparatus: DSC7020/manufactured by Hitachi High-Tech Science Corporation
Heating rate: 10° C./min.
Measurement temperature range: 30° C. to 350° C.
Measurement atmosphere: nitrogen 50 mL/min.
Measurement sample: synthesized epoxy resin composition 3 mg

### 3. Measurement of Glass Transition Temperature (Tg) (Dynamic Viscoelastic Measurement (DMA))

Apparatus: DMA850/manufactured by TA Instruments Japan
Measurement conditions: three-point bending
Measurement temperature: 30° C. to 310° C.
Measurement frequency: 1.0 (Hz)
Sample size: (60 mm×15 mm×2 mm)
Heating rate: 1.0° C./min.

### 4. Dielectric Properties Evaluation

Films (sample size: width 1.5 mm, length 8.0 mm) prepared in Examples and Comparative Examples were measured for relative dielectric constant and dielectric loss tangent using the following apparatus (sample size: width 1.5 mm, length 8.0 mm).
Measuring apparatus: PNA network analyzer N522B (manufactured by Keysight Technologies)
Cavity resonator: CP531 for 10 GHZ (manufactured by Kanto Electronics Application & Development Inc.)

### [Measurement Conditions]

Test method: in accordance with IEC 62180 (cavity resonator perturbation method)
Test conditions: frequency; 10 GHZ
Number of measurements: n=2

### <Example 1> (Synthesis of Ester Compound (p-151) Represented by Chemical Formula 1-1 Below)

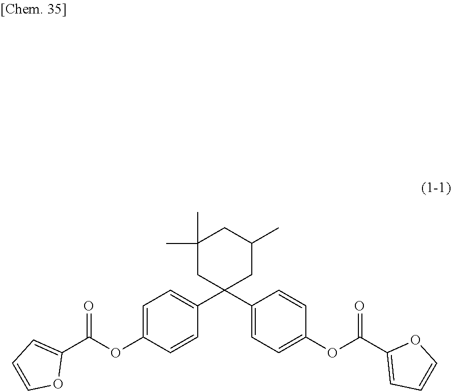 [SMILES: CC1CC(C)(C)CC(c2ccc(OC(=O)c3ccco3)cc2)(c2ccc(OC(=O)c3ccco3)cc2)C1]

A 1000 mL four-necked flask equipped with a thermometer, a stirrer, a condenser, and a dropping funnel was charged with 465 g (1.50 mol) of bisphenol TMC and 352 g of acetic anhydride, and after the reaction vessel was purged with nitrogen, the temperature of the mixed solution was brought to 130° C. Thereafter, stirring was performed at 130° C. for 6 hours. The composition of the reaction solution was analyzed by UFLC according to the above analysis method, revealing that the proportion of 1,1-bis(4-acetoxyphenyl)-3,3,5-trimethylcyclohexane present in the reaction solution was 96 area %. After completion of the reaction, acetic anhydride and produced acetic acid were removed by distillation under reduced pressure at 130° C. The pressure during the distillation was gradually reduced so as to finally reach 1.5 kPa. A composition containing 1,1-bis(4-acetoxyphenyl)-3,3,5-trimethylcyclohexane was obtained in an amount of 560 g (purity: 99.9%).

The results of 1H-NMR analysis confirmed that 1,1-bis(4-acetoxyphenyl)-3,3,5-trimethylcyclohexane was obtained.

¹H-NMR analysis (400 MHz, solvent: CDCl₃, reference material: tetramethylsilane)

0.40 (3H, s), 0.85-0.89 (1H, t), 0.96-0.97 (6H, d), 1.14-1.20 (1H, t), 1.37-1.40 (1H, d), 1.56 (2H, s), 1.92-2.10 (2H, m), 2.25-2.27 (6H, d), 2.42-2.45 (1H, d), 2.64-2.68 (1H, d), 6.90-6.92 (2H, d), 6.98-7.00 (2H, d), 7.18-7.20 (2H, d), 7.31-7.34 (2H, d).

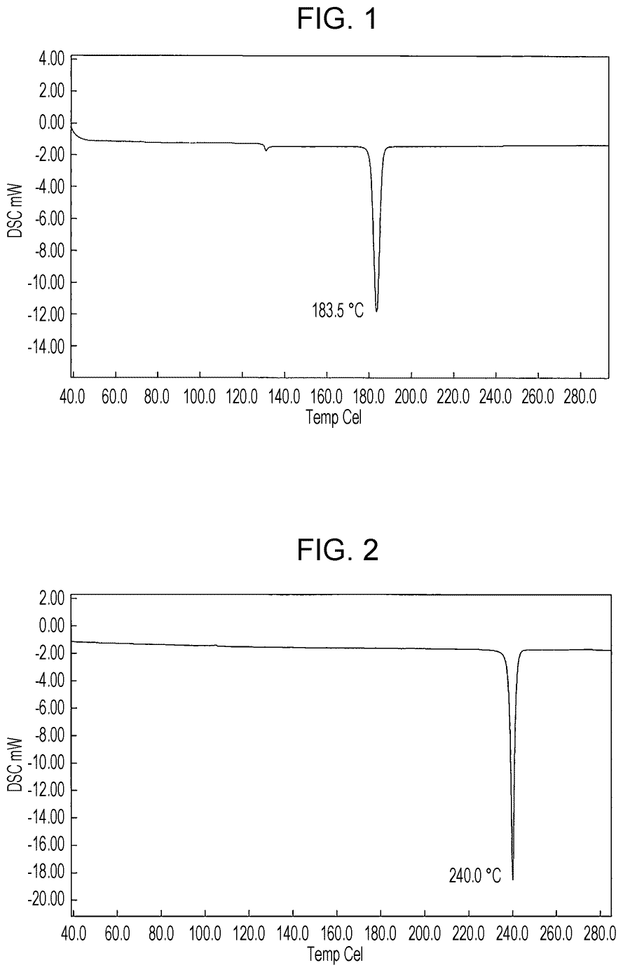

A 3000 mL four-necked flask equipped with a thermometer, a stirrer, a condenser, and a dropping funnel was charged with 300 g (0.76 mol) of 1,1-bis(4-acetoxyphenyl)-3,3,5-trimethylcyclohexane, 213 g of furancarboxylic acid, 4.0 g of 4-dimethylaminopyridine, and 1500 g of diphenyl ether, and after the reaction vessel was purged with nitrogen, the temperature of the mixed solution was brought to 230° C. Thereafter, stirring was performed at 230° C. for 8 hours while a liquid containing acetic acid produced as the reaction proceeded, diphenyl ether as a solvent, and others was distilled out of the system. The composition of the reaction solution was analyzed by UFLC according to the above analysis method, revealing that the proportion of the target compound present in the reaction solution was 52 area %. After completion of the reaction, produced acetic acid, diphenyl ether, and furancarboxylic acid were removed by distillation under reduced pressure at 210° C. The pressure during the distillation was gradually reduced so as to finally reach 5.0 kPa. The concentrated diphenyl ether solution containing the target compound was cooled, and then 845 g of 1-butanol was added. Thereafter, the resultant was cooled to 70° C. to precipitate a crystal. 1-Butanol in an amount of 229 g was added, and the resultant was cooled to 30° C. The resulting slurry containing the target compound was subjected to solid-liquid separation by centrifugal filtration, and the resulting solvent-containing crystal was dried at 80° C. and 2.0 kPa to obtain 337 g of the target compound (1-1) (purity: 99.8%). As a result of differential scanning calorimetry (DSC), the target compound was shown to be a crystal having a maximum endothermic peak temperature of 183.5° C. The DSC data are shown in FIG. 1.

The results of 1H-NMR analysis confirmed that the target compound having the above structure was obtained.

¹H-NMR analysis (400 MHz, solvent: CDCl₃, reference material: tetramethylsilane)

0.39 (3H, s), 0.81-0.92 (1H, t), 0.98-1.00 (6H, d), 1.18-1.24 (1H, t), 1.39-1.42 (1H, d), 1.56 (4H, s), 1.96-2.10 (2H, m), 2.46-2.49 (1H, d), 2.68-2.72 (1H, d), 6.57-6.59 (2H, m), 7.04-7.06 (2H, d), 7.13-7.15 (2H, d), 7.24-7.33 (1H, m), 7.34-7.40 (4H, m), 7.65-7.67 (2H, m).

For the solvent solubility of the obtained target compound, it exhibits a solubility of 20 to 40 wt % relative to the weight of each of the solutions in methyl ethyl ketone, cyclohexanone, and N-methylpyrrolidone and a solubility of 10 to 20 wt % relative to the weight of each of the solutions in toluene, propylene glycol monomethyl ether acetate, and ethyl acetate, and thus has been confirmed to have excellent solvent solubility.

### <Example 2> (Synthesis of Ester Compound (p-28) Represented by Chemical Formula 1-2 Below)

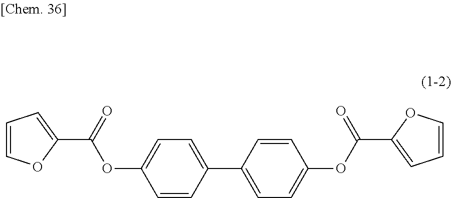 [SMILES: O=C(Oc1ccc(-c2ccc(OC(=O)c3ccco3)cc2)cc1)c1ccco1]

After diacetyl biphenol was synthesized by acetylating biphenol in the same manner as in Example 1, a 1000 mL four-necked flask equipped with a thermometer, a stirrer, a condenser, and a dropping funnel was charged with 70 g (0.26 mol) of diacetyl biphenol, 72 g of furancarboxylic acid, 1.4 g of 4-dimethylaminopyridine, and 431 g of diphenyl ether, and after the reaction vessel was purged with nitrogen, the temperature of the mixed solution was brought to 210° C. Thereafter, stirring was performed at 210° C. for 4 hours while a liquid containing acetic acid produced as the reaction proceeded, diphenyl ether as a solvent, and others was distilled out of the system under reduced-pressure conditions. The pressure during the distillation was gradually reduced so as to finally reach 25.0 kPa. The composition of the reaction solution was analyzed by UFLC according to the above analysis method, revealing that the proportion of the target compound present in the reaction solution was 69 area %. After completion of the reaction, the resultant was cooled to 30° C. to precipitate a crystal. The resulting slurry containing the target compound was subjected to solid-liquid separation by centrifugal filtration, and the resulting solvent-containing crystal was dried at 80° C. and 2.0 kPa to obtain 81 g of the target compound (1-2) (purity: 99.7%). As a result of differential scanning calorimetry (DSC), the target compound was shown to be a crystal having a maximum endothermic peak temperature of 240.0° C. The DSC data are shown in FIG. 2.

The results of 1H-NMR analysis confirmed that the target compound having the above structure was obtained.

¹H-NMR analysis (400 MHz, solvent: CDCl₃, reference material: tetramethylsilane)

6.82-6.83 (2H, dd), 7.37-7.40 (4H, dt), 7.60-7.61 (2H, dd), 7.76-7.80 (4H, dt), 8.13 (2H, s).

### <Example 3> (Synthesis of Ester Compound (p-148) Represented by Chemical Formula 1-3 Below)

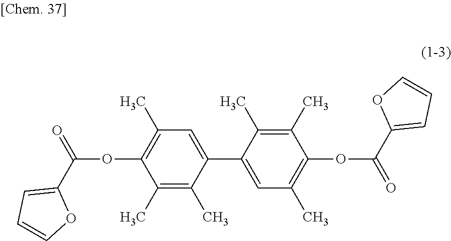 [SMILES: Cc1cc(-c2cc(C)c(OC(=O)c3ccco3)c(C)c2C)c(C)c(C)c1OC(=O)c1ccco1]

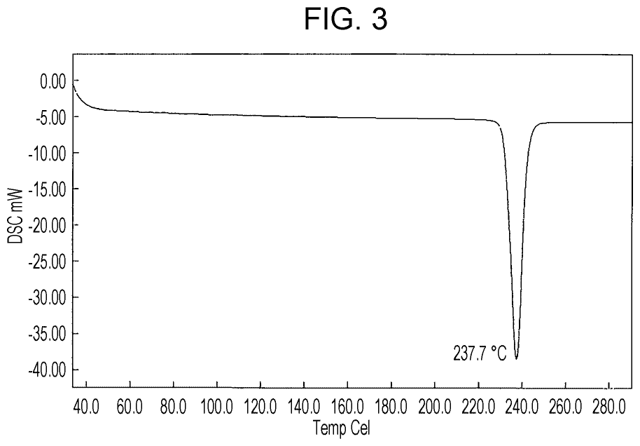

After 4,4′-diacetoxy-2,2′,3,3′,5,5′-hexamethylbiphenyl was synthesized by acetylating 4,4′-dihydroxy-2,2′,3,3′,5,5′-hexamethylbiphenyl in the same manner as in Example 1, a 2000 mL four-necked flask equipped with a thermometer, a stirrer, a condenser, and a dropping funnel was charged with 204 g (0.58 mol) of 4,4′-diacetoxy-2, 2 ‘, 3, 3’, 5,5′-hexamethylbiphenyl, 162 g of furancarboxylic acid, 3.8 g of 4-dimethylaminopyridine, and 1267 g of diphenyl ether, and after the reaction vessel was purged with nitrogen, the temperature of the mixed solution was brought to 220° C. Thereafter, stirring was performed at 220° C. for 14 hours while a liquid containing acetic acid produced as the reaction proceeded, diphenyl ether as a solvent, and others was distilled out of the system. The composition of the reaction solution was analyzed by UFLC according to the above analysis method, revealing that the proportion of the target compound present in the reaction solution was 70 area %. After completion of the reaction, produced acetic acid, diphenyl ether, and furancarboxylic acid were removed by distillation under reduced pressure at 220° C. The pressure during the distillation was gradually reduced so as to finally reach 5.0 kPa. The concentrated diphenyl ether solution containing the target compound was cooled, and then 88 g of isopropanol and 149 g of diphenyl ether were added. Thereafter, the resultant was cooled to 30° C. to precipitate a crystal. The resulting slurry containing the target compound was subjected to solid-liquid separation by centrifugal filtration, and the resulting solvent-containing crystal was dried at 60° C. and 2.0 kPa to obtain 233 g of the target compound (purity: 99.4%). As a result of differential scanning calorimetry (DSC), the target compound was shown to be a crystal having a maximum endothermic peak temperature of 237.7° C. The DSC data are shown in FIG. 3.

The results of 1H-NMR analysis confirmed that the target compound having the above structure was obtained.

¹H-NMR analysis (400 MHz, solvent: C₂D₆OS, reference material: tetramethylsilane)

1.94 (3H, s), 2.09 (6H, s), 2.11 (6H, s), 6.84-6.85 (2H, dd), 6.93 (2H, s), 7.67-7.68 (2H, dd), 8.15-8.15 (2H, d).

### Example 4

In a 100 ml beaker, 10.0 g of the compound (1-1) synthesized in Example 1, 10.1 g of a dicyclopentadiene-type epoxy resin (XD-1000, manufactured by Nippon Kayaku Co., Ltd., epoxy equivalent 251 g/eq.), 0.2 g of 4-dimethylaminopyridine, and 16.2 g of methyl ethyl ketone were loaded and dissolved by heating at 70° C. The solution was cast on a release film and air-dried at room temperature. Thereafter, the resultant was dried at 80° C. and 1.5 kPa with a vacuum dryer and then crushed to obtain a composition powder including the compound (1-1) and the epoxy resin. The composition powder was packed in a mold made of silicone resin, and cured at 140° C./1 hour, 150° C./1 hour, 160° C./1.5 hours, 180° C./1.5 hours, and 250° C./2 hours. The glass transition point (Tg) of the obtained cured product was measured by the above analysis method and found to be 250.0° C.

### Example 5

In a 100 ml beaker, a mixture of 5.1 g of the compound (1-1) synthesized in Example 1 and 5.0 g of 1,1-bis(4-acetoxyphenyl)-3,3,5-trimethylcyclohexane synthesized in Example 1, 11.4 g of a dicyclopentadiene-type epoxy resin (XD-1000, manufactured by Nippon Kayaku Co., Ltd., epoxy equivalent 251 g/eq.), 0.2 g of 4-dimethylaminopyridine, and 11.1 g of methyl ethyl ketone were loaded and dissolved by heating at 70° C. The solution was cast on a release film and air-dried at room temperature. Thereafter, the resultant was dried at 80° C. and 1.5 kPa with a vacuum dryer and then crushed to obtain a composition powder including the compound (1-1), 1,1-bis(4-acetoxyphenyl)-3,3,5-trimethylcyclohexane, and the epoxy resin. The composition powder was packed in a mold made of silicone resin, and cured at 140° C./1 hour, 150° C./1 hour, 160° C./1.5 hours, and 180° C./1.5 hours. The glass transition point (Tg) of the obtained cured product was measured by the above analysis method and found to be 206.3° C.

Examples 4 and 5 have revealed that a higher proportion of the compound represented by general formula (1) provides higher heat resistance.

### Example 6

In a 100 ml beaker, 20.0 g of the compound (1-1) synthesized in Example 1, 16.0 g of an ortho-cresol novolac-type epoxy resin (trade name “EPICLON N-673” manufactured by DIC Corporation), and 40.0 g of methyl ethyl ketone were loaded and dissolved by stirring. To the solution, 0.72 g of 4-dimethylaminopyridine was added, and stirring was further performed to dissolve 4-dimethylaminopyridine. After complete dissolution, the solution was transferred to a tray and dried overnight in a draft chamber, and then dried at 60° C. and 1.5 kPa for 5 hours with a vacuum dryer. Thereafter, the obtained composition was put in a mold (p 100 mm, positive mold) and heated with a hot press tester under the conditions of 160° C./2 hours and 180° C./2 hours at 3 MPa to obtain a cured product.

### Comparative Example 1

In a 100 ml beaker, 10.0 g of a novolac-type curing agent (trade name “BRG-555” manufactured by Aika Kogyo Co., Ltd.), 20.0 g of an ortho-cresol novolac-type epoxy resin (trade name “EPICLON N-673” manufactured by DIC Corporation), and 40.0 g of methyl ethyl ketone were loaded and dissolved by stirring. To the solution, 0.60 g of triphenylphosphine was added, and stirring was further performed to dissolve triphenylphosphine. After complete dissolution, the solution was transferred to a tray and dried overnight in a draft chamber, and then dried at 60° C. and 1.5 kPa for 5 hours with a vacuum dryer. Thereafter, the obtained composition was put in a mold (p 100 mm, positive mold) and heated with a hot press tester under the conditions of 100° C./1 hour and 130° C./2 hours at 3 MPa. Thereafter, the cured product was heated with a hot air circulating oven under the conditions of 140° C./2 hours, 150° C./2 hours, 160° C./2 hours, and 180° C./2 hours to obtain a cured product.

The cured products obtained in Example 6 and Comparative Example 1 were evaluated for glass transition temperature (Tg) and dielectric properties by the above analysis methods. The results are listed in Table 1.

TABLE 1

| | Glass | Relative | |
|---|---|---|---|
| | transition | dielectric | Dielectric |
| | temperature | constant | loss tangent |
| Example 6 | 157° C. | 3.16 | 0.0270 |
| Comparative | 140° C. | 3.15 | 0.0485 |
| Example 1 | | | |

As shown above, the epoxy resin cured product obtained using the compound of Example 1, which is the inventive compound, as a curing agent was shown to have a high glass transition temperature and exhibit high heat resistance, and was also shown to exhibit excellent dielectric properties.

The ester compound according to the present invention, when mixed with an epoxy resin and cured, provides a cured product having excellent heat resistance and dielectric properties. It is presumed that for a secondary hydroxy group formed as a result of the reaction between an epoxy resin and a curing agent, the ester compound represented by general formula (1) reacts with the epoxy resin as a curing agent and esterifies the secondary hydroxy group, whereby a cured product that is less polarized and that exhibits excellent dielectric properties is provided.

Thus, the epoxy resin composition containing the ester compound according to the present invention is applicable to various fields, including adhesives, coatings, materials for civil engineering and construction, and insulating materials for electric and electronic components, and is useful particularly as an insulating casting, a laminating material, a sealing material, etc. in the electric and electronic fields.

Examples of applications of the ester compound according to the present invention and the epoxy resin composition containing the ester compound include multilayer printed circuit boards, laminates for electric and electronic circuits such as capacitors, adhesives such as film adhesives and liquid adhesives, semiconductor sealing materials, underfill materials, inter-chip fills for 3D-LSI, insulating sheets, prepregs, and heat dissipation substrates, but are not limited thereto.

1. An ester compound represented by general formula (3): 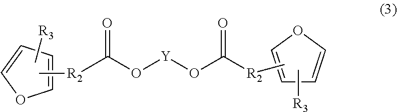
wherein each R₂ independently represents a single bond or a divalent hydrocarbon group having 1 to 10 carbon atoms, each R₃ independently represents a hydrogen atom or an alkyl group having 1 to 6 carbon atoms, and Y represents a divalent group represented by general formula (3a) or general formula (3b); 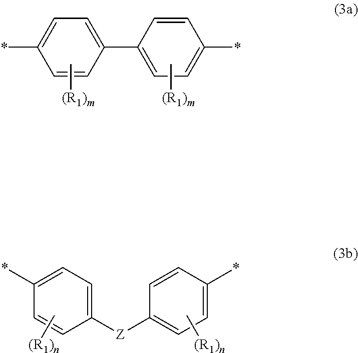
wherein each R₁ independently represents an alkyl group having 1 to 6 carbon atoms or an aryl group having 6 to 12 carbon atoms, and each * represents a bonding position; in general formula (3a), each m independently represents an integer of 1 to 4; and in general formula (3b), each n independently represents 0 or an integer of 1 to 4, and Z represents a cycloalkylidene group having 7 to 20 carbon atoms.

2. The ester compound according to claim 1, wherein each R₂ of the ester compound represented by general formula (3) is a single bond.

3. The ester compound according to claim 2, wherein furthermore, each R₃ of the ester compound represented by general formula (3) is a hydrogen atom.

4. The ester compound according to claim 3, wherein the ester compound represented by general formula (3) is compound (p-139), (p-142), (p-145), (p-148), (p-151), (p-154), or (p-172):

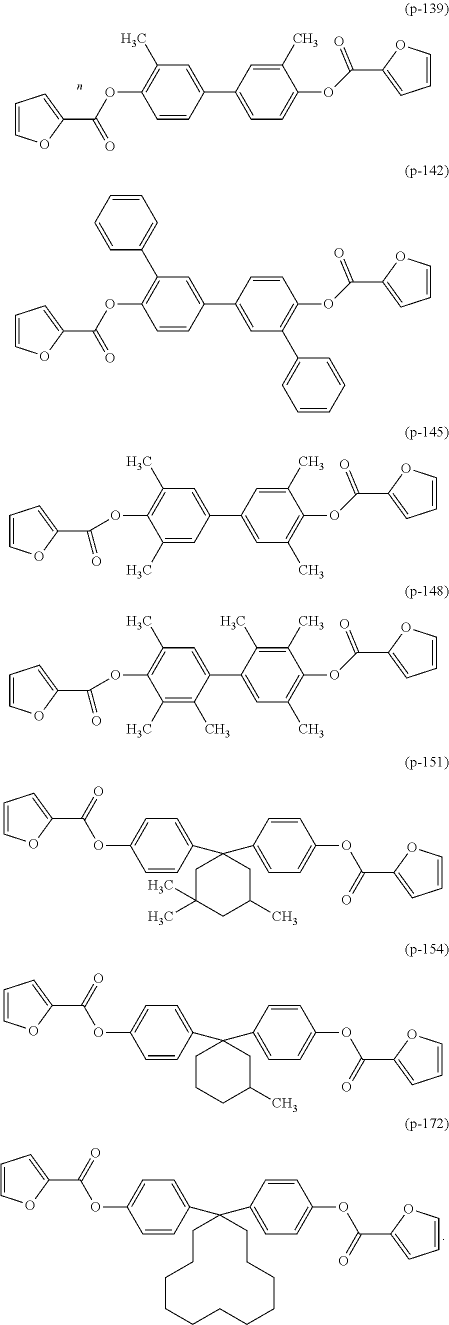

5. A crystal of the ester compound according to claim 4, the ester compound being compound (p-148).

6. The crystal of the ester compound according to claim 5, the ester compound being compound (p-148), wherein a maximum endothermic peak temperature determined by differential scanning calorimetry is in a range of 234° C. to 240° C.

7. A crystal of the ester compound according to claim 4, the ester compound being compound (p-151).

8. The crystal of the ester compound according to claim 7, the ester compound being compound (p-151), wherein a maximum endothermic peak temperature determined by differential scanning calorimetry is in a range of 180° C. to 188° C.

9. An ester compound composition for a resin raw material, comprising the ester compound represented by general formula (3) according to claim 1 and an ester compound represented by general formula (2): 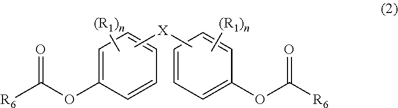
wherein each R₁ independently represents an alkyl group having 1 to 6 carbon atoms or an aryl group having 6 to 12 carbon atoms, each R₆ independently represents a monovalent hydrocarbon group having 1 to 20 carbon atoms, X represents a single bond, an oxygen atom, a sulfur atom, a sulfonyl group, a carbonyl group, or a divalent group represented by general formula (1a), (1b), or (1c), and each n independently represents 0 or an integer of 1 to 4: 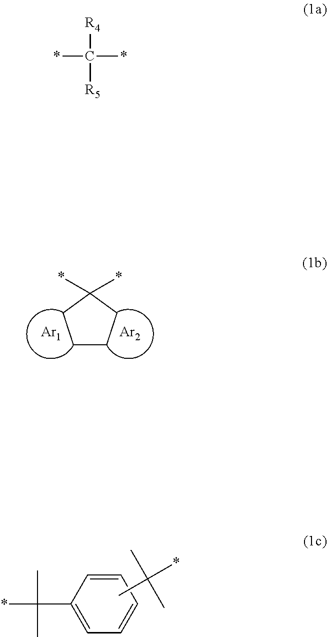
wherein R₄ and R₅ each independently represent a hydrogen atom, an alkyl group having 1 to 10 carbon atoms, an alkyl halide group having 1 to 10 carbon atoms, or an aryl group having 6 to 12 carbon atoms, R₄ and R₅ may be bonded to each other to together form a cycloalkylidene group having 5 to 20 carbon atoms, Ar₁ and Ar₂ represent an aryl group having 6 to 12 carbon atoms, and each * represents a bonding position.

10. The ester compound composition for a resin raw material according to claim 9, wherein the ester compound represented by general formula (2) is contained in an amount of 0.1 to 400 parts by weight relative to 100 parts by weight of the ester compound represented by general formula (3).

11. An epoxy resin formed by reacting the ester compound represented by general formula (3) according to claim 1 with any one selected from an aromatic diglycidyl ether compound, a mixture containing an aromatic dihydroxy compound and epihalohydrin, and an epoxy group-containing phenoxy resin formed by polymerizing an aromatic dihydroxy compound and epihalohydrin.
